(19) United States
(12) Patent Application Publication
(10) Pub. No.: US 2014/0056842 A1
(43) Pub. Date: Feb. 27, 2014

(54) CELLULAR THERAPEUTIC APPROACHES TO TRAUMATIC BRAIN AND SPINAL CORD INJURY

(71) Applicants: Jonathan Sackner-Bernstein, Dobbs Ferry, NY (US); Andrew L. Pecora, Wycoff, NJ (US); Robert A. Preti, Ridgefield, NJ (US)

(72) Inventors: Jonathan Sackner-Bernstein, Dobbs Ferry, NY (US); Andrew L. Pecora, Wycoff, NJ (US); Robert A. Preti, Ridgefield, NJ (US)

(21) Appl. No.: 13/792,368

(22) Filed: Mar. 11, 2013

Related U.S. Application Data
(60) Provisional application No. 61/691,665, filed on Aug. 21, 2012.

(51) Int. Cl. A61K 38/19; A61K 38/18; A61K 35/14

(52) U.S. Cl. CPC A61K 38/193; A61K 35/14; A61K 38/191; A61K 38/1866
USPC 424/85.1; 424/93.7

(57) ABSTRACT

The described invention provides cellular therapeutic approaches for treating a vascular insufficiency following a traumatic injury to head or spine that results in an injury to brain, spinal cord, or both by administering a therapeutic amount of an isolated, nonexpanded population of autologous mononuclear cells comprising a subpopulation of CD34+ cells, which further contains a subpopulation of potent SDF-1 mobile CD34+/CXCR-4 cells that have CXCR-4-mediated chemotactic activity.

### CROSS-REFERENCE TO RELATED APPLICATIONS

This application claims priority from U.S. provisional patent application Ser. No. 61/691,665, filed Aug. 21, 2012, the entire disclosure of which is incorporated herein by reference.

### FIELD OF THE INVENTION

The invention relates to pharmaceutical compositions and methods to treat traumatic brain injury.

### BACKGROUND OF THE INVENTION

### 1. Central Nervous System

The central nervous system is a bilateral and essentially symmetrical structure with seven main parts: the spinal cord, medulla oblongata, pons, cerebellum, midbrain, diencephalon, and the cerebral hemispheres. FIG. 1 shows a lateral view of the human brain from Stedman's Medical Dictionary, 27^{th} Edition, plate 7 at A7 (2000).

The spinal cord, the most caudal part of the central nervous system, receives and processes sensory information from the skin, joints, and muscles of the limbs and trunk and controls movement of the limbs and the trunk. It is subdivided into cervical, thoracic, lumbar and sacral regions. The spinal cord comprises gray matter and surrounding white matter. The gray matter, which contains nerve cell bodies, is typically divided into dorsal and ventral horns. The dorsal horn contains an orderly arrangement of sensory relay neurons that receive input from the periphery, while the ventral horn contains motor nuclei that innervate specific muscles.

The spinal cord continues rostrally as the brainstem, which consists of the medulla, pons, and midbrain. The brainstem receives sensory information from the skin and muscles of the head and provides the motor control for the muscles of the head. It also conveys information from the spinal cord to the brain and from the brain to the spinal cord, and regulates levels of arousal and awareness through the reticular formation. The brainstem contains several collections of cell bodies, the cranial nerve nuclei. Some of these receive information from the skin and muscles of the head; others control motor output to muscles of the face, neck and eyes. Still others are specialized for information from the special senses: hearing, balance and taste. (Kandel, E. et al., Principles of Neural Science, 4th Ed., p. 8, 2000).

The medulla oblongata, which lies directly rostral to the spinal cord, includes several centers responsible for vital autonomic functions, such as digestion, breathing and the control of heart rate (Kandel, E. et al., Principles of Neural Science, 4th Ed., p. 8, 2000).

The pons, which lies rostral to the medulla, conveys information about movement from the cerebral hemispheres to the cerebellum (Kandel, E. et al., Principles of Neural Science, 4th Ed., p. 8, 2000).

The cerebellum lies behind the pons and is connected to the brain stem by several major fiber tracts called peduncles. The cerebellum modulates the force and range of movement, and is involved in the learning of motor skills. It also contributes to learning and cognition (Kandel, E. et al., Principles of Neural Science, 4th Ed., p. 8, 2000).

The midbrain, which lies rostral to the pons, controls many sensory and motor functions, including eye movements and the coordination of visual and auditory reflexes (Kandel, E. et al., Principles of Neural Science, 4th Ed., p. 8, 2000).

The diencephalon lies rostral to the midbrain and contains two structures. One, the thalamus, processes most of the information reaching the cerebral cortex from the rest of the central nervous system and is involved in other functions including motor control, autonomic function and cognition. The other, the hypothalamus, regulates autonomic, endocrine, and visceral function (Kandel, E. et al., Principles of Neural Science, 4th Ed., p. 8, 2000).

The cerebral hemispheres consist of a heavily wrinkled outer layer, the cerebral cortex, and deep-lying gray-matter structures—the basal ganglia, which participate in regulating motor performance; the hippocampus, which is involved with aspects of learning and memory storage; and the amygdaloid nuclei, which coordinate the autonomic and endocrine responses of emotional states (Kandel, E. et al., Principles of Neural Science, 4th Ed., p. 8, 2000).

The cerebral cortex is divided into four lobes: the frontal lobe, parietal lobe, temporal lobe and occipital lobe. The surfaces of the cerebral hemispheres contain many grooves or furrows, known as fissures and sulci. The portions of brain lying between these grooves are called convolutions or gyri. The lateral cerebral fissure (fissure of Sylvius) separates the temporal from the frontal lobe. The central sulcus (Rolandic sulcus) separates the frontal from the parietal lobe (Kandel, E. et al., Principles of Neural Science, 4th Ed., p. 8, 2000).

2. Circulation of the Brain

FIGS. 2, 3, 4 and 5 show schematic illustrations of the brain's blood vessels. Each cerebral hemisphere is supplied by an internal carotid artery, which arises from a common carotid artery beneath the angle of the jaw, enters the cranium through the carotid foramen, traverses the cavernosus sinus (giving off the ophthalmic artery), penetrates the dura and divides into the anterior and middle cerebral arteries. The large surface branches of the anterior cerebral artery supply the cortex and white matter of the inferior frontal lobe, the medial surface of the frontal and parietal lobes and the anterior corpus callosum. Smaller penetrating branches supply the deeper cerebrum and diencephalon, including limbic structures, the head of the caudate, and the anterior limb of the internal capsule. The large surface branches of the middle cerebral artery supply most of the cortex and white matter of the hemisphere's convexity, including the frontal, parietal, temporal and occipital lobes, and the insula. Smaller penetrating branches supply the deep white matter and diencephalic structures such as the posterior limb of the internal capsule, the putamen, the outer globus pallidus, and the body of the caudate. After the internal carotid artery emerges from the cavernous sinus, it also gives off the anterior choroidal artery, which supplies the anterior hippocampus and, at a caudal level, the posterior limb of the internal capsule. Each vertebral artery arises from a subclavian artery, enters the cranium through the foramen magnum, and gives off an anterior spinal artery and a posterior inferior cerebellar artery. The vertebral arteries join at the junction of the pons and the medulla to form the basilar artery, which at the level of the pons gives off the anterior inferior cerebellar artery and the internal auditory artery, and, at the midbrain, the superior cerebellar artery. The basilar artery then divides into the two posterior cerebral arteries. The large surface branches of the posterior cerebral arteries supply the inferior temporal and medial occipital lobes and the posterior corpus callosum; the smaller penetrating branches of these arteries supply diencephalic structures, including the thalamus and the subthalamic nuclei, as well as part of the midbrain (see Principles of Neural Sciences, 2d Ed., Eric R. Kandel and James H. Schwartz, Elsevier Science Publishing Co., Inc., New York, pp. 854-56 (1985)).

Interconnections between blood vessels (anastomoses) protect the brain when part of its vascular supply is compromised. Anastomoses are interconnections between blood vessels that protect the brain when part of its vascular supply is compromised. At the circle of Willis, the two anterior cerebral arteries are connected by the anterior communicating artery and the posterior cerebral arteries are connected to the internal carotid arteries by the posterior communicating arteries. Other important anastomoses include connections between the ophthalmic artery and branches of the external carotid artery through the orbit, and connections at the brain surface between branches of the middle, anterior, and posterior cerebral arteries (Principles of Neural Sciences, 2d Ed., Eric R. Kandel and James H. Schwartz, Elsevier Science Publishing Co., Inc., New York, pp. 854-56 (1985)).

The circle of Willis at the base of the brain is the principal arterial anastomotic trunk of the brain. Blood reaches it mainly via the vertebral and internal carotid arteries (See FIG. 4); anastomoses occur between arterial branches of the circle of Willis over the cerebral hemispheres and via extracranial arteries that penetrate the skull through various foramina.

The circle of Willis is formed by anastamoses between the internal carotid, basilar, anterior cerebral, anterior communicating, posterior cerebral, and posterior communicating arteries. The internal carotid artery terminates in the anterior cerebral and middle cerebral arteries. Near its termination, the internal carotid artery gives rise to the posterior communicating artery, which joins caudally with the posterior cerebral artery. The anterior cerebral arteries connect via the anterior communicating artery.

Venous drainage from the brain chiefly is into the dural sinuses, vascular channels lying within the tough structure of the dura. The dural sinuses contain no valves and, for the most part, are triangular in shape. The superior longitudinal sinus is in the falx cerebri.

The human brain constitutes only about 2% of the total weight of the body, but it receives about 15% of cardiac output, and its oxygen consumption is approximately 20% of that for the total body. These values indicate the high metabolic rate and oxygen requirement of the brain that are compensated by a correspondingly high rate of blood flow per unit brain weight. Cerebral circulation is supplied by the internal carotid arteries and the vertebral arteries. The total blood flow to the brain is about 750-1000 ml/min; of this amount about 350 ml flows through each internal carotid artery and about 100-200 ml flows through the vertebral basilar system. The venous outflow is drained by the internal jugular veins and the vertebral veins.

Cerebral vessels are capable of altering their own diameter and can respond in a unique fashion to altered physiological conditions. Two main types of autoregulation exist that help maintain optimal cerebral blood flow. Brain arterioles constrict when the systemic blood pressure is raised and dilate when it is lowered. The result is that normal individuals have a constant cerebral blood flow between mean arterial pressure of approximately 60-150 mm Hg. The second type of autoregulation involves blood or tissue gases and pH. When arterial CO2 is raised, brain arterioles dilate and cerebral blood flow decreases. Changing arterial O2 causes an opposite and less pronounced response. These responses protect the brain by increasing the delivery of oxygen and the removal of acid metabolites in the presence of hypoxia, ischemia, or tissue damage. They also allow instantaneous adjustments of regional cerebral blood flow to meet the demands of rapidly changing oxygen and glucose metabolism that accompany normal brain activities.

### 3. Cerebral Arteries

The blood supply to the cerebral cortex mainly is via cortical branches of the anterior cerebral, middle cerebral, and posterior cerebral arteries, which reach the cortex in the pia mater. FIG. 3 shows an illustrative view of the arterial supply of the cerebral cortex where 1 is the orbitofrontal artery; 2 is the prerolandic artery; 3 is the rolandic artery; 4 is the anterior parietal artery; 5 is the posterior parietal artery; 6 is the angular artery; 7 is the posterior temporal artery; 8 is the anterior temporal artery; 9 is the orbital artery; 10 is the frontopolar artery; 11 is the callosomarginal artery; 12 is the posterior internal frontal artery; and 13 is the pericallosal artery (Correlative Neuroanatomy & Functional Neurology, 18th Ed., p. 50, 1982).

The lateral surface of each cerebral hemisphere is supplied mainly by the middle cerebral artery. The medial and inferior surfaces of the cerebral hemispheres are supplied by the anterior cerebral and posterior cerebral arteries.

The anterior cerebral artery supplies the medial portions of the frontal and parietal lobes and corpus callosum. The middle cerebral artery supplies large portions of the frontal, parietal, and temporal lobe surfaces. Branches of the anterior and middle cerebral arteries (lenticulostriate arteries) supply the basal ganglia and anterior limb of the internal capsule.

The middle cerebral artery, a terminal branch of the internal carotid artery, enters the lateral cerebral fissure and divides into cortical branches that supply the adjacent frontal, temporal, parietal and occipital lobes. Small penetrating arteries, the lenticulostriate arteries, arise from the basal portion of the middle cerebral artery to supply the internal capsule and adjacent structures.

The anterior cerebral artery extends medially from its origin from the internal carotid artery into the longitudinal cerebral fissure to the genu of the corupus callosum, where it turns posteriorly close to the corpus callosum. It gives branches to the medial frontal and parietal lobes and to the adjacent cortex along the medial surface of these lobes.

The posterior cerebral artery arises from the basilar artery at its rostral end usually at the level of the midbrain, curves dorsally around the cerebral peduncle, and sends branches to the medial and inferior surfaces of the temporal lobe and to the medial occipital lobe. Branches include the calcarine artery and perforating branches to the posterior thalamus and subthalamus.

The basilar artery is formed by the junction of the vertebral arteries. It supplies the upper brain stem via short paramedian, short circumferential, and long circumferential branches.

The midbrain is supplied by the basilar, posterior cerebral, and superior cerebellar arteries. The pons is supplied by the basilar, anterior cerebellar, inferior cerebellar, and superior cerebellar arteries. The medulla oblongata is supplied by the vertebral, anterior spinal, posterior spinal, posterior inferior cerebellar, and basilar arteries. The cerebellum is supplied by the cerebellar arteries (superior cerebellar, anterior inferior cerebellar, and posterior inferior cerebellar arteries).

The choroid plexuses of the third and lateral ventricles are supplied by branches of the internal carotid and posterior cerebral arteries. The choroid plexus of the fourth ventricle is supplied by the posterior inferior cerebellar arteries.

The vertebral and basilar arteries supply the brain stem, cerebellum, posterior cerebral cortex, and medial temporal lobe. The posterior cerebral arteries bifurcate from the basilar artery to supply the medial temporal (including the hippocampus) and occipital lobes, thalamus, and mammillary and geniculate bodies.

### 4. Spinal Cord Circulation

The ventral horn of the spinal cord is supplied by a single anterior spinal artery. The dorsal horn of the spinal cord is supplied by two or more posterior spinal arteries.

The anterior spinal artery is formed by the midline union of paired branches of the vertebral arteries and extends along the anterior surface of the cervical spinal cord, narrowing near the upper (fourth) thoracic segments.

The lateral spinal arteries arise as a single set of branches from the vertebral arteries and pass through the lower cervical and upper thoracic intervertebral foramens to supply the spinal cord segments.

The anterior medial spinal artery is the prolongation of the anterior spinal artery below the fourth thoracic cord segment. Intercostal arteries from the aorta supply segmental branches to the spinal cord to the level of the first lumbar cord segment. The largest of these branches, the great ventral radicular artery, enters the spinal cord between the eight thoracic and fourth lumbar cord segments. This large artery, also known as the arteria radicularis magna, or artery of Adamkiewicz.

In the lumbosacral area, radicular arteries are derived from the lumbar, iliolumbar, and lateral sacral arteries. The major such vessels appear to enter the intervertebral foramens at the second lumbar vertebra to form the lowermost portion of the anterior spinal artery, called the terminal artery, which runs along the filum terminale.

The posterior spinal arteries, also known as posterolateral spinal arteries, receive branches from the posterolateral arterial plexus at various levels. They are paired and are considerably smaller than the single large anterior spinal artery.

Anterior sulcal arteries arise from the anterior spinal artery at various levels along the cervical and thoracic cord within the anterior sulcus and supply the anterior and lateral columns on either side of the spinal cord. The posterial spinal arteries supply the posterior white columns and the more posterior part of the posterior gray columns.

Segmentally, arteries that enter the intervertebral foramens are given off from the intercostal vessels and lateral sacral arteries. Arteries are also given off from the dorsal and ventral radicular arteries, which accompany the posterior and anterior nerve roots, respectively. These unite directly with the posterior and anterior spinal arteries and then are joined together segmentally along the periphery of the spinal cord as the arteriae coronae.

### 5. Conditions Involving Disruption of Blood Circulation

Conditions involving the blood vessels are among the most frequent serious neurological disorders, ranking third as a cause of death in the adult population in the United States and probably first as a cause of chronic functional incapacity. (Principles of Neural Sciences, 2d Ed., Eric R. Kandel and James H. Schwartz, Elsevier Science Publishing Co., Inc., New York, pp. 854-56 (1985); and The Merck Manual, Health Care Professionals, Neurologic Disorders, Stroke (CVA), accessed on Aug. 1, 2012).

The term “stroke” or “cerebrovascular accident” as used herein refers to neurological symptoms and signs, usually focal and acute, that result from diseases involving blood essels.of the CNS Generally, strokes are either ischemic (about 80%, resulting from thrombosis or embolism) or hemorrhagic (about 20%, due to bleeding from a blood vessel resulting from a vascular rupture).

The term “ischemia” as used herein refers to a lack of blood supply and oxygen that occurs: (a) when reduced perfusion pressure distal to an abnormal narrowing (stenosis) of a blood vessel is not compensated by autoregulatory dilation of the resistance vessels; (b) by a thrombus forming in situ; or (c) by an embolus becoming lodged within a vessel. When ischemia is sufficiently severe and prolonged, neurons and other cellular elements of brain tissue die; this condition is referred to as “infarction.” Stroke symptoms lasting less than 1 h are termed a transient ischemic attack (TIA); TIAs often do not damage brain tissue, and when damage occurs, it is less extensive than that due to strokes.

Hemorrhage may occur at the brain surface (extraparenchymal), for example from the rupture of congenital aneurysms at the circle of Willis, causing subarachnoid hemorrhage (SAH). Hemorrhage also may be intraparenchymal, for example from rupture of vessels damaged by long-standing hypertension, and may cause a blood clot (intracerebral hematoma) within the cerebral hemispheres, in the brain stem, or in the cerebellum. Hemorrhage may be accompanied by ischemia or infarction. The mass effect of an intracerebral hematoma may compromise the blood supply of adjacent brain tissue; or subarachnoid hemorrhage may cause reactive vasospasm of cerebral surface vessels, leading to further ischemic brain damage. Infarcted tissue may also become secondarily hemorrhagic. Aneurysms occasionally can rupture into the brain, causing an intracerebral hematoma, and into the cerebral ventricles, causing intraventricular hemorrhage.

Although most occlusive strokes are due to atherosclerosis and thrombosis and most hemorrhagic strokes are associated with hypertension or aneurysms, strokes of either type may occur at any age from many causes, including, without limitation: cardiac disease, trauma, infection, neoplasm, blood dyscrasia, vascular malformation, immunological disorder, and exogenous toxins.

Strokes involve either the anterior circulation (branches of the internal carotid artery) or the posterior circulation (branches of the vertebral and basilar arteries) of the brain. Infarction occurring in the middle cerebral artery territory causes the most frequently encountered stroke syndrome, with contralateral weakness, sensory loss, and visual field impairment, and depending on the hemisphere involved, either language disturbance or impaired spatial perception. Infarction in the territory of the anterior cerebral artery causes weakness, sensory loss, and urinary incontinence. Damage to the supplementary motor cortex may cause speech disturbance. Bilateral anterior cerebral artery territory infarction (occurring for example when both arteries arise anomalously from a single trunk) may cause severe behavioral disturbance, known as abulia, consisting of profound apathy, motor inertia and muteness, attributable to destruction of inferior frontal lobes of the orbitofrontal cortex. Infarction in the territory of the posterior cerebral artery causes contralateral homonymous hemianopsia by destroying the calcarine cortex. Anterior choroidal artery occlusion can cause contralateral hemiplegia and sensory loss. Infarctions restricted to the structures supplied by the deep penetrating branches of the anterior cerebral artery result in varying combinations of psychomotor slowing, dysarthia, agitation, contralateral neglect, and when left hemispheric, language disturbance. Atherothrombotic vessel occlusion can also occur in the internal carotid artery rather than intracranial vessels. Infarctions may include the territories of both the middle and anterior cerebral arteries with arm and leg weakness and sensory loss. Alternatively, infarctions may be limited to the distal shared territory (border zones of these vessels), producing, by destruction of the motor cortex, weakness limited to the arm or the leg.

Risk factors for stroke may include but are not limited to: prior stroke, age, family history, alcoholism, male sex, hypertension, cigarette smoking, hypercholesterolemia, diabetes, use of certain drugs, such as cocaine, amphetamines, etc. Certain risk factors predispose to a particular type of stroke (eg, hypercoagulability predisposes to thrombotic stroke, atrial fibrillation to embolic stroke, and intracranial aneurysms to subarachnoid hemorrhage).

Initial stroke symptoms may be sudden and may include but are not limited to numbness, weakness, or paralysis of the contralateral limbs and the face; aphasia; confusion; visual disturbances in one or both eyes (eg, transient monocular blindness); dizziness or loss of balance and coordination; and headache.

Neurologic deficits reflect the area of the brain involved. Anterior circulation stroke typically causes unilateral symptoms. Posterior circulation stroke can cause unilateral or bilateral deficits and is more likely to affect consciousness, especially when the basilar artery is involved. Table 1 summarizes selected stroke syndromes and the associated symptoms and signs.

TABLE 1
Selected Stroke Syndromes

| Syndrome | Symptoms and Signs |
|---|---|
| Anterior cerebral artery | Contralateral hemiparesis (maximal in the leg), urinary |
| (uncommon) | incontinence, apathy, confusion, poor judgment, mutism, |
| | grasp reflex, gait apraxia |
| Middle cerebral artery (common) | Contralateral hemiparesis (worse in the arm and face than |
| | in the leg), dysarthria, hemianesthesia, contralateral |
| | homonymous hemianopia, aphasia (if the dominant |
| | hemisphere is affected) or apraxia and sensory neglect (if |
| | the nondominant hemisphere is affected) |
| Posterior cerebral artery | Contralateral homonymous hemianopia, unilateral cortical |
| | blindness, memory loss, unilateral 3rd cranial nerve palsy, |
| | hemiballismus |
| Ophthalmic artery (a branch of the | Monocular loss of vision (amaurosis) |
| middle cerebral artery) | |
| Vertebrobasilar system | Unilateral or bilateral cranial nerve deficits (eg, |
| | nystagmus, vertigo, dysphagia, dysarthria, diplopia, |
| | blindness), truncal or limb ataxia, spastic paresis, crossed |
| | sensory and motor deficits*, impaired consciousness, |
| | coma, death (if basilar artery occlusion is complete), |
| | tachycardia, labile BP |
| | *Ipsilateral facial sensory loss or motor weakness with |
| | contralateral body hemianesthesia or hemiparesis indicates |
| | a lesion at the pons or medulla. |
| Lacunar infarcts | Absence of cortical deficits plus one of the following: |
| | Pure motor hemiparesis |
| | Pure sensory hemianesthesia |
| | Ataxic hemiparesis |
| | Dysarthria-clumsy hand syndrome |

Manifestations other than neurologic deficits often suggest the type of stroke. For example, sudden, severe headache suggests subarachnoid hemorrhage. Impaired consciousness or coma, often accompanied by headache, nausea, and vomiting, suggest increased intracranial pressure, which can occur 48 to 72 h after large ischemic strokes and earlier with many hemorrhagic strokes; fatal brain herniation may result.

If stroke is suspected, immediate neuroimaging is required to differentiate hemorrhagic from ischemic stroke and to detect signs of increased intracranial pressure. Computed tomography (CT) is sufficiently sensitive to detect intracranial blood but may be normal or show only subtle changes during the first hours of symptoms after anterior circulation ischemic stroke. CT also misses some small posterior circulation strokes and up to 3% of subarachnoid hemorrhages. CT can usually be done more rapidly, but magnetic resonance imaging (MRI) is sensitive for intracranial blood and may detect signs of ischemic stroke missed by CT. If CT does not confirm clinically suspected stroke, diffusion-weighted MRI can usually detect ischemic stroke. After the stroke is identified as ischemic or hemorrhagic, tests are done to determine the cause. Patients are also evaluated for coexisting acute general disorders (eg, infection, dehydration, hypoxia, hyperglycemia, hypertension).

Treatment of stroke is two fold: stabilization and supportive measures and treatment of complications. Stabilization may need to precede complete evaluation. Comatose or obtunded patients (eg, Glasgow Coma Score ≦8) may require airway support. If increased intracranial pressure is suspected, intracranial pressure monitoring and measures to reduce cerebral edema may be necessary. Specific acute treatments vary by type of stroke.

Providing supportive care, correcting coexisting abnormalities (eg, fever, hypoxia, dehydration, hyperglycemia, sometimes hypertension), and preventing and treating complications are vital during the acute phase and convalescence. Strategies to prevent and treat stroke complications may include but are not limited to: applying tight elastic or air-filled support stockings and providing frequent active and passive leg exercises; turning bedridden patients frequently, with special attention to pressure sites; passively moving limbs at risk of contractures and placing them in the appropriate resting positions, using splints if necessary; ensuring adequate fluid intake and nutrition, including evaluating patients for swallowing difficulties and providing nutritional support as necessary; giving small doses of heparin or heparinoid, when not contraindicated, to prevent deep venous thromobosis and pulmonary embolism; encouraging early ambulation (as soon as vital signs are normal), with close monitoring; maximizing lung function (eg, smoking cessation, deep breathing exercises, respiratory therapy, measures to prevent aspiration in patients with dysphagia); looking for and treating infections early, especially pneumonia, urinary tract infections, and skin infections; managing urinary bladder problems in bedbound patients, preferably without using an indwelling catheter; promoting risk factor modification (eg, smoking cessation, weight loss, healthful diet); prescribing early rehabilitation (eg, active and passive exercises, range-of-motion exercises); discussing residual function, prognosis for recovery, and strategies to compensate for lost function with the patient; encouraging maximum independence through rehabilitation; and encouraging patient and family members to contact social stroke support groups for social and psychologic support.

### 6. Ishemic Stroke

Ischemic stroke is a sudden neurologic deficit that results from focal cerebral ischemia associated with permanent brain infarction (eg, positive diffusion-weighted MRI). Common causes are (from most to least common) nonthrombotic occlusion of small, deep cortical arteries (lacunar infarction); cardiogenic embolism; arterial thrombosis that decreases cerebral blood flow; and artery-to-artery embolism. Ischemic stroke is usually diagnosed clinically. A computed tomography (CT) or Magnetic Resonance Imaging (MRI) scan is usually done to exclude hemorrhage and confirm the presence and extent of stroke. Thrombolytic therapy may be useful acutely in certain patients. Depending on the cause of stroke, carotid endarterectomy, or antiplatelet drugs (e.g. aspirin) or anticoagulants (e.g., warfarin) may help reduce risk of subsequent strokes. (The Merck Manual, Health Care Professionals, Neurologic Disorders, Stroke (CVA) accessed on Aug. 1, 2012).

Etiology

Ischemia usually results from thrombi or emboli. Even infarcts classified as lacunar based on clinical criteria (morphology, size, and location) often involve small thrombi or emboli.

Thrombosis:

Atheromas, particularly if ulcerated, predispose to thrombi. Atheromas can occur in any major cerebral artery and are common at areas of turbulent flow, particularly at the carotid bifurcation. Partial or complete thrombotic occlusion occurs most often at the main trunk of the middle cerebral artery and its branches but is also common in the large arteries at the base of the brain, in deep perforating arteries, and in small cortical branches. The basilar artery and the segment of the internal carotid artery between the cavernous sinus and supraclinoid process are often occluded. Less common causes of thrombosis include vascular inflammation secondary to disorders such as acute or chronic meningitis, vasculitic disorders, and syphilis; dissection of intracranial arteries or the aorta; hypercoagulability disorders (eg, antiphospholipid syndrome, hyperhomocysteinemia); hyperviscosity disorders (eg, polycythemia, thrombocytosis, hemoglobinopathies, plasma cell disorders); and rare disorders (eg, moyamoya disease, Binswanger's disease). Older oral contraceptive formulations increase risk of thrombosis.

Embolism:

Emboli may lodge anywhere in the cerebral arterial tree. Emboli may originate as cardiac thrombi, especially in the following conditions: atrial fibrillation, rheumatic heart disease, post-myocardial infarctions, vegetations on heart valves in bacterial or merantic endocarditis, prosthetic heart valves, etc. Other sources include clots that form after open-heart surgery and atheromas in neck arteries or in the aortic arch. Rarely, emboli consist of fat (from fractured long bones), air (in decompression sickness), or venous clots that pass from the right to the left side of the heart through a patent foramen ovale with shunt (paradoxical emboli). Emboli may dislodge spontaneously or after invasive cardiovascular procedures (eg, catheterization). Rarely, thrombosis of the subclavian artery results in embolic stroke in the vertebral artery or its branches.

Lacunar Infarcts:

Ischemic stroke can also result from lacunar infarcts. These small (≦1.5 cm) infarcts result from nonatherothrombotic obstruction of small, perforating arteries that supply deep cortical structures; the usual cause is lipohyalinosis (degeneration of the media of small arteries and replacement by lipids and collagen). Whether emboli cause lacunar infarcts is controversial. Lacunar infarcts tend to occur in elderly patients with diabetes or poorly controlled hypertension.

Other Causes:

Less commonly, ischemic stroke results from vasospasm (eg, during migraine, after subarachnoid hemorrhage, after use of sympathomimetic drugs such as cocaine or amphetamines) or venous sinus thrombosis (eg, during intracranial infection, postoperatively, peripartum, secondary to a hypercoagulation disorder).

Pathophysiology

Inadequate blood flow in a single brain artery can often be compensated for by an efficient collateral system, particularly between the carotid and vertebral arteries via anastomoses at the circle of Willis and, to a lesser extent, between major arteries supplying the cerebral hemispheres. However, normal variations in the circle of Willis and in the caliber of various collateral vessels, atherosclerosis, and other acquired arterial lesions can interfere with collateral flow, increasing the chance that blockage of one artery will cause brain ischemia.

Some neurons die when perfusion is <5% of normal for >5 min; however, the extent of damage depends on the severity of ischemia. If it is mild, damage proceeds slowly; thus, even if perfusion is 40% of normal, 3 to 6 h may elapse before brain tissue is completely lost. However, if severe ischemia (ie, decrease in perfusion) persists >15 to 30 min, all of the affected tissue dies (infarction). Damage occurs more rapidly during hyperthermia and more slowly during hypothermia. If tissues are ischemic but not yet irreversibly damaged, promptly restoring blood flow may reduce or reverse injury. For example, intervention may be able to salvage the moderately ischemic areas (penumbras) that often surround areas of severe ischemia (these areas exist because of collateral flow).

Mechanisms of ischemic injury include edema, microvascular thrombosis, programmed cell death (apoptosis), and infarction with cell necrosis. Inflammatory mediators (eg, IL-1β, tumor necrosis factor-α) contribute to edema and microvascular thrombosis. Edema, if severe or extensive, can increase intracranial pressure. Many factors may contribute to necrotic cell death; they include loss of ATP stores, loss of ionic homeostasis (including intracellular Ca accumulation), lipid peroxidative damage to cell membranes by free radicals (an iron-mediated process), excitatory neurotoxins (eg, glutamate), and intracellular acidosis due to accumulation of lactate.

Symptoms and Signs

Symptoms and signs depend on the part of brain affected. Patterns of neurologic deficits often suggest the affected artery (Table 1). Deficits may become maximal within several minutes of onset, typically in embolic stroke. Less often, deficits evolve slowly, usually over 24 to 48 h (called evolving stroke or stroke in evolution), typically in thrombotic stroke. In most evolving strokes, unilateral neurologic dysfunction (often beginning in one arm, then spreading ipsilaterally) extends without causing headache, pain, or fever. Progression is usually stepwise, interrupted by periods of stability. A stroke is considered submaximal when, after it is complete, there is residual function in the affected area, suggesting viable tissue at risk of damage.

Embolic strokes often occur during the day; headache may precede neurologic deficits. Thrombi tend to occur during the night and thus are first noticed on awakening. Lacunar infarcts may produce one of the classic lacunar syndromes (eg, pure motor hemiparesis, pure sensory hemianesthesia, ataxic hemiparesis, dysarthria-clumsy hand syndrome); signs of cortical dysfunction (eg, aphasia) are absent. Multiple lacunar infarcts may result in multi-infarct dementia.

Deterioration during the first 48 to 72 h after onset of symptoms, particularly progressively impaired consciousness, results more often from cerebral edema than from extension of the infarct. Unless the infarct is large or extensive, function commonly improves within the first few days; further improvement occurs gradually for up to 1 yr.

Diagnosis

Diagnosis is suggested by sudden neurologic deficits referable to a specific arterial territory. Ischemic stroke must be distinguished from other causes of similar focal deficits (eg, hypoglycemia; postictal [Todd's] paralysis; hemorrhagic stroke; rarely, migraine). Headache, coma or stupor, and vomiting are more likely with hemorrhagic stroke.

Although diagnosis is clinical, neuroimaging and bedside glucose testing are mandatory. CT is done first to exclude intracerebral hemorrhage, subdural or epidural hematoma, and a rapidly growing, bleeding, or suddenly symptomatic tumor. CT evidence of even large anterior circulation ischemic stroke may be subtle during the first few hours; changes may include effacement of sulci or the insular cortical ribbon, loss of the gray-white junction between cortex and white matter, and a dense middle cerebral artery sign. After 24 h of ischemia, medium-sized to large infarcts are usually visible as hypodensities; small infarcts (eg, lacunar infarcts) may be visible only with MRI. Diffusion-weighted MRI (highly sensitive for early ischemia) can be done immediately after CT initial neuroimaging.

Distinction between lacunar, embolic, and thrombotic stroke based on history, examination, and neuroimaging is not always reliable, so tests to identify common or treatable causes and risk factors for all of these types of strokes are routinely done. These tests typically include carotid duplex ultrasonography, ECG, transesophageal echocardiography, and various blood tests (CBC, platelet count, PT/PTT, fasting blood glucose, lipid profile, homocysteine, ESR, and, for at-risk patients, syphilis serology). Troponin I level is measured to detect concomitant MI. Magnetic resonance or CT angiography is also often done. Other tests (eg, antiphospholipid antibodies) are done if certain disorders are suspected clinically.

Prognosis

Stroke severity and progression are often assessed using standardized measures such as the National Institutes of Health Stroke Scale (NIHSS) (Table 2). The NIHSS score correlates with extent of functional impairment and prognosis. During the first days, progression and outcome can be difficult to predict. Older age, impaired consciousness, aphasia, and brain stem signs suggest a poor prognosis. Early improvement and younger age suggest a favorable prognosis.

TABLE 2
The National Institutes of Health Stroke Scale
Table 2. The National Institutes of Health Stroke Scale

| Criterion | Finding | Score |
|---|---|---|
| 1a. Level of | Alert: keenly responsive | 0 |
| Consciousness | Not alert; but arousable by minor | 1 |
| | stimulation to obey, answer, | |
| | or respond. | |
| | Not alert; requires repeated | 2 |
| | stimulation to attend, or is | |
| | obtunded and requires strong | |
| | or painful stimulation to make | |
| | movements (not stereotyped). | |
| | Responds only with reflex motor | 3 |
| | or autonomic effects, or totally | |
| | unresponsive, flaccid, and | |
| | areflexic. | |
| 1b. Level of | Answers both questions correctly | 0 |
| Consciousness | Answers one question correctly. | 1 |
| The patient is asked the | Answers neither question | 2 |
| month and his/her age | correctly. | |
| 1c. Level of | Performs both tasks correctly. | 0 |
| Consciousness | Performs one task correctly. | 1 |
| The patient is asked to | Performs neither task correctly. | 2 |
| open and close | | |
| the eyes and then to grip | | |
| and release the non- | | |
| paretic hand. | | |
| 2. Best Gaze | Normal | 0 |
| Only horizontal eye | Partial gaze palsy; gaze is | 1 |
| movements tested | abnormal | |
| | in one or both eyes, but forced | |
| | deviation or total gaze paresis is | |
| | not present. | |
| | Forced deviation, or total | 2 |
| | gaze paresis is not overcome by the | |
| | oculocephalic maneuver. | |
| 3. Visual | No visual loss. | 0 |
| Visual fields (upper and | Partial hemianopia. | 1 |
| lower quadrants) are | Complete hemianopia. | 2 |
| tested by confrontation, | Bilateral hemianopia (blind | 3 |
| using finger counting | including | |
| or visual threat, as | cortical blindness). | |
| appropriate. | | |
| 4. Facial Palsy | Normal symmetrical movements. | 0 |
| Patient asked or | Minor paralysis (flattened | 1 |
| encouraged to show teeth | nasolabial | |
| or raise eyebrows and | fold, asymmetry on smiling). | |
| close eyes. | Partial paralysis (total or near- | 2 |
| | total | |
| | paralysis of lower face). | |
| | Complete paralysis of one or both | 3 |
| | sides (absence of facial movement | |
| | in | |
| | the upper and lower face). | |
| 5. Motor Arm | No drift; limb holds 90 (or 45) | 0 |
| 5a Left Arm | degrees | |
| 5b Right Arm | for full 10 seconds. | |
| The limb is placed in the | Drift; limb holds 90 (or 45) | 1 |
| appropriate position: | degrees, | |
| extend the arms (palms | but drifts down before full 10 | |
| down) 90 degrees | seconds; | |
| (if sitting) or 45 degrees | does not hit bed or other support. | |
| (if supine). Drift is | Some effort against gravity; limb | 2 |
| scored if the arm falls | cannot get to or maintain (if cued) | |
| before 10 seconds. | 90 | |
| | (or 45) degrees, drifts down to bed, | |
| | but has some effort against gravity. | |
| | No effort against gravity; limb | 3 |
| | falls. | |
| | No movement. | 4 |
| | Amputation or joint fusion | UN |
| 6. Motor Leg | No drift; leg holds 30-degree | 0 |
| 6a Left Leg | position | |
| 6b Right Leg | for full 5 seconds. | |
| The limb is placed in the | Drift; leg falls by the end of the 5- | 1 |
| appropriate position: | second period but does not hit the | |
| hold the leg at 30 degrees | bed. | |
| (always tested | Some effort against gravity; leg | 2 |
| supine). Drift is scored if | falls | |
| the leg falls before | to bed by 5 seconds but has some | |
| 5 seconds. | effort against gravity. | |
| | No effort against gravity; leg falls | 3 |
| | to | |
| | bed immediately. | |
| | No movement. | 4 |
| | Amputation or joint fusion, | UN |
| | explain: | |
| 7. Limb Ataxia | Absent. | 0 |
| aimed at finding evidence | Present in one limb. | 1 |
| of a | Present in two limbs. | 2 |
| unilateral cerebellar | Amputation or joint fusion | UN |
| lesion | | |
| 8. Sensory | Normal; no sensory loss. | 0 |
| Sensation or grimace to | Mild-to-moderate sensory loss; | 1 |
| pinprick when tested, | patient feels pinprick is less sharp | |
| or withdrawal from | or is dull on the affected side; or | |
| noxious stimulus in the | there is a loss of superficial pain | |
| obtunded or aphasic | with pinprick, but patient is aware | |
| patient. | of being touched | |
| | Severe or total sensory loss; | 2 |
| | patient | |
| | is not aware of being touched in the | |
| | face, arm, and leg. | |
| 9. Best Language | No aphasia; normal. | 0 |
| patient is asked to | Mild-to-moderate aphasia; some | 1 |
| describe what is | obvious | |
| happening in the attached | loss of fluency or facility of | |
| picture, to name the items | comprehension, | |
| on a | without significant limitation on | |
| naming sheet, and to read | ideas | |
| from a | expressed or form of expression. | |
| list of sentences. | Severe aphasia; all | 2 |
| | communication is | |
| | through fragmentary expression; | |
| | Mute, global aphasia; no usable | 3 |
| | speech | |
| | or auditory comprehension. | |
| 10. Dysarthria | Normal. | 0 |
| If patient is thought to be | Mild-to-moderate dysarthria; | 1 |
| normal, an | patient slurs at least some words | |
| adequate sample of | and, at worst, can be understood | |
| speech must be obtained | with some difficulty. | |
| by asking patient to read | Severe dysarthria; patient's | 2 |
| or repeat words from | speech | |
| a list. | is so slurred as to be unintelligible | |
| | in the absence of or out of | |
| | proportion to any dysphasia, or is | |
| | mute/anarthric. | |
| | Intubated or other physical barrier | UN |
| 11. Extinction and | No abnormality. | 0 |
| Inattention (formerly | Visual, tactile, auditory, spatial, | 1 |
| neglect) | or | |
| Sufficient information to | personal inattention, or extinction | |
| identify neglect may | to | |
| be obtained during the | bilateral simultaneous stimulation | |
| prior testing. | in | |
| | one of the sensory modalities. | |
| | Profound hemi-inattention or | 2 |
| | extinction | |
| | to more than one modality; does | |
| | not | |
| | recognize own hand or orients to | |
| | only one | |
| | side of space. | |

About 50% of patients with moderate or severe hemiplegia and most with milder deficits have a clear sensorium and eventually can take care of their basic needs and walk adequately. Complete neurologic recovery occurs in about 10%. Use of the affected limb is usually limited, and most deficits that remain after 12 mo are permanent. Subsequent strokes often occur, and each tends to worsen neurologic function. About 20% of patients die in the hospital; mortality rate increases with age.

Treatment

Treatment of ischemic stroke includes but is not limited to general stroke treatments, acute hypertensive therapy, antiplatelet therapy, tissue plasminogen activator in case of acute treatment, anticoagulation, long term control of risk factors and carotid endarterectomy.

Patients with acute ischemic strokes are usually hospitalized. Supportive measures be needed during initial evaluation and stabilization. Perfusion of an ischemic brain area may require a high blood pressure (BP) because autoregulation is lost; thus, BP should not be decreased except in the following situations: (i) BP is >220 mm Hg systolic or >120 mm Hg diastolic on 2 successive readings >15 min apart; (ii) signs of other end-organ damage (eg, aortic dissection, acute MI, pulmonary edema, hypertensive encephalopathy, retinal hemorrhages, acute renal failure); (iii) use of recombinant tissue plasminogen activator (tPA) is likely. If indicated, nicardipine or labetalol are initially administered intravenously. Patients with presumed thrombi or emboli may be treated with tPA, thrombolysis-in-situ, antiplatelet drugs, and/or anticoagulants. Most patients are not candidates for thrombolytic therapy; they should be given an antiplatelet drug (eg. aspirin).

Recombinant tPA is used for patients with acute ischemic stroke of <3 h duration and no contraindications to tPA. However, tPA can cause fatal or other symptomatic brain hemorrhage and can only be administered by highly experienced physicians. Anticoagulants and antiplatelet drugs are not used within 24 h of treatment with tPA.

Thrombolysis-in-situ (angiographically directed intra-arterial thrombolysis) of a thrombus or embolus can sometimes be used for major strokes if symptoms have begun >3 h but <6 h ago, particularly for strokes due to large occlusions in the middle cerebral artery. Clots in the basilar artery may be intra-arterially lysed up to 12 h after stroke onset, sometimes even later depending on the clinical circumstances. This treatment, although standard of care in some large stroke centers, is often unavailable in other hospitals.

Anticoagulation therapy with heparin is used for stroke caused by cerebral venous thrombosis and is sometimes used for emboli due to atrial fibrillation and when stroke due to presumed progressive thrombosis continues to evolve despite use of antiplatelet drugs and cannot be treated any other way (eg, with tPA or invasive methods). Warfarin is also used simultaneously with heparin.

Long term: Supportive care is continued during convalescence. Controlling general medical risk factors (especially hyperglycemia and fever) can limit brain damage after stroke, leading to better functional outcomes.

Carotid endarterectomy is indicated for patients with recent nondisabling, submaximal stroke attributed to an ipsilateral carotid obstruction of 70 to 99% of the arterial lumen or to an ulcerated plaque if life expectancy is at least 5 yr. In other symptomatic patients (eg, patients with TIAs), endarterectomy with antiplatelet therapy is indicated for carotid obstruction of >60% with or without ulceration if life expectancy is at least 5 yr. The procedure should be done by surgeons who have a morbidity and mortality rate of <3% with the procedure in the hospital where it will be done. If carotid stenosis is asymptomatic, endarterectomy is beneficial only when done by very experienced surgeons, and that benefit is likely to be small. For many patients, carotid stenting with an emboli-protection device (a type of filter) is as effective as surgery.

Oral antiplatelet drugs are often used to prevent subsequent strokes (secondary prevention). Aspirin (81 or 325 mg/day), clopidogrel 75 mg once/day, or the combination product aspirin 25 mg/extended-release dipyridamole 200 mg bid may be used.

### 7. Transient Ishemic Attack (TIA)

The term “transient ischemic attack” (TIA) as used herein refers to a focal brain ischemia that causes sudden neurologic deficits and is not associated with permanent brain infarction (eg, negative results on diffusion-weighted MRI). Diagnosis is clinical. Carotid endarterectomy, antiplatelet drugs, and warfarin decrease risk of stroke after certain types of TIA.

TIA is similar to ischemic stroke except that symptoms last <1 h; most TIAs last <5 min. Infarction is very unlikely if deficits resolve within 1 h. Deficits that resolve spontaneously within 1 to 24 h have been shown on diffusion-weighted MRI and other studies to often be accompanied by infarction and are thus no longer considered TIAs. TIAs are most common among the middle-aged and elderly. TIAs markedly increase risk of stroke, beginning in the first 24 h.

Etiology

Most TIAs are caused by emboli, usually from carotid or vertebral arteries, although most of the causes of ischemic stroke can also result in TIAs. Uncommonly, TIAs result from impaired perfusion due to severe hypoxemia, reduced O2-carrying capacity of blood (eg, profound anemia, carbon monoxide poisoning), or increased blood viscosity (eg, severe polycythemia), particularly in brain arteries with preexisting stenosis. Systemic hypotension does not usually cause cerebral ischemia unless it is severe or arterial stenosis preexists because autoregulation maintains brain blood flow at near-normal levels over a wide range of systemic BPs.

In subclavian steal syndrome, a subclavian artery stenosed proximal to the origin of the vertebral artery “steals” blood from the vertebral artery (in which blood flow reverses) to supply the arm during exertion, causing signs of vertebrobasilar ischemia.

Symptoms

Neurologic deficits are similar to those of strokes (Table 1). Transient monocular blindness (amaurosis fugax), which usually lasts <5 min, may occur when the ophthalmic artery is affected. Symptoms begin suddenly, usually last 2 to 30 min, then resolve completely. Patients may have several TIAs daily or only 2 or 3 over several years. Symptoms are usually similar in successive carotid attacks but vary somewhat in successive vertebrobasilar attacks.

Diagnosis

Diagnosis is made retrospectively when sudden neurologic deficits referable to ischemia in an arterial territory resolve within 1 h. Isolated peripheral facial nerve palsy, loss of consciousness, or impaired consciousness does not suggest TIA. TIAs must be distinguished from other causes of similar symptoms (eg, hypoglycemia, migraine aura, postictal [Todd's] paralysis). Because an infarct, a small hemorrhage, and even a mass lesion cannot be excluded clinically, neuroimaging is required. Usually, CT is the study most likely to be immediately available. However, CT may not identify infarcts for >24 h. MRI usually detects evolving infarction within hours. Diffusion-weighted MRI is the most accurate imaging test to rule out an infarct in patients with presumed TIA but is not always available.

The cause of a TIA is sought as for that of ischemic strokes, including tests for carotid stenosis, cardiac sources of emboli, atrial fibrillation, and hematologic abnormalities and screening for stroke risk factors.

Treatment

Treatment is aimed at preventing strokes; antiplatelet drugs are used. Carotid endarterectomy or arterial angioplasty plus stenting can be useful for some patients, particularly those who have no neurologic deficits but who are at high risk of stroke. Warfarin is indicated if cardiac sources of emboli are present. Modifying stroke risk factors, when possible, may prevent stroke.

### 8. Traumatic Brain Injury (TBI)

Traumatic brain injury (TBI) occurs as the result of a direct mechanical insult to the brain and induces degeneration and death in the central nervous system (CNS). Following the initial mechanical insult, secondary pathways are activated, which contribute to the ischemic damage induced by the circulatory disturbance, blood-brain barrier disruption, and excitotoxicity damage. These findings suggest that CNS disorders can be caused by widespread neuronal and axonal degeneration induced by TBI.

Emerging evidence also points to long-term sequelae on a broad range of neurologic and psychiatric functions associate with TBI. For example, patients with relatively modest closed head trauma often present with subtle initial clinical findings, yet still develop symptoms ranging from headache and blurred vision to confusion, change in behavioral, and/or impaired memory/concentration. Neuroimaging can detect parenchymal bleeding, which is insufficient to warrant surgical intervention, yet indicative of an ongoing process of tissue injury including disruption of the blood brain barrier and mass effects that can affect cerebral parenchyma more broadly than may be apparent from first examination. Such concerns rise with more extensive injury and more violent forces.

According to the Centers for Disease Control (CDC), an estimated 1.7 million people sustain a TBI annually. TBI is a contributing factor to a third (30.5%) of all injury-related deaths in the United States. About 75% of TBIs that occur each year are concussions or other forms of mild TBI. Direct medical costs and indirect costs, such as lost productivity, of TBI totaled an estimated $76.5 billion in the United States in 2000.

Many who experience mild or mild-to-moderate closed head trauma do not undergo routine neuroimaging, as is the case for teen or professional sports injuries as well as motor vehicle accidents. While more severe injuries can require craniectomies, even recurrent concussive trauma, viewed individually as relatively minor, increases the risk of chronic traumatic encephalopathy (CTE), a tau protein-linked neurodegenerative disease.

The onset of CTE relative to the severity of injury in the general population has been postulated to be consistent with that seen in the military, and certainly the publicity around the deaths of some professional athletes, and the lawsuits filed against the National Football League, raises considerable concerns. Military medicine is collaborating with civilian providers to better understand brain injury through the National Intrepid Center of Excellence (NICoE) that provides training to non-military physicians and health-care providers in strategies developed for military applications for TBI patients, thus bridging the gap between civilian and military TBI care.

A recent study suggests that the long term consequences of brain injuries are similar for soldiers, football players and others involved in impact sports such as ice hockey, boxing and wrestling. Furthermore, the classification of TBI is inadequate so that there is poor correlation between the initial allocation of severity and subsequent pathologies. Concussive events, whether caused by a blast or by repeated blows, can lead to the development of chronic traumatic encephalopathy (CTE), a tau protein-linked neurodegenerative disease, suggesting a mechanistic link between such concussive events and persistent impairments in neurophysiological function and learning and memory, which are the hallmarks of chronic TBI. (Goldstein L E, et al., Sci. Transl. Med. 4:134, 2012).

Critical care management of severe traumatic brain injury in adults is reviewed in Haddad, S. H. et al., Scandinavian Journal of Trauma, Resuscitation and Emergency Medicine, 20: 12, pages 1-15 (2012), incorporated herein by reference.

Those suffering from traumatic brain injury (TBI) may experience transient or subclinical effects, which are now recognized as carrying the potential for long-term sequelae. Current knowledge regarding pathophysiology of cerebral ischemia and brain trauma indicates that similar mechanisms contribute to loss of cellular integrity and tissue destruction. Mechanisms of cell damage include, but are not limited to, excitotoxicity, oxidative stress, free radical production, apoptosis, and inflammation.

In addition, severe cerebral ischemic insults lead to metabolic stress, ionic perturbations, and a complex cascade of biochemical and molecular events ultimately causing neuronal death. In the setting of a brief (e.g., lasting three minutes to five minutes) coronary artery, energy metabolism is impaired, leading to ionic perturbations, and a complex cascade of biochemical and molecular events ultimately causing neuronal death.

### 9. Inflammatory and Apoptotic Mechanisms of Ischemia

Inflammatory and apoptotic mechanisms following an ischemic injury have been studied extensively in the field of acute myocardial infarction (AMI).

Preclinical and clinical data demonstrate that following a myocardial infarction, the acute loss of myocardial muscle cells and the accompanying peri-infarct zone hypo-perfusion result in a cascade of events causing an immediate diminution of cardiac function, with the potential for long term persistence. The extent of myocardial cell loss is dependent on the duration of coronary artery occlusion, existing collateral coronary circulation and the condition of the cardiac microvasculature. Paul et al., Am. Heart J. 131: 710-15 (1996); Pfeffer, M. A., Braunwald, E., Circulation 81: 1161-72 (1990); Sheiban, I. e. al., J. Am. Coll. Cardiol. 38: 464-71 (2001); Braunwald E., Bristow, M. R., Circulation 102: IV-14-23 (2000); Rich et al., Am. J. Med. 92:7-13 (1992); Ren et al., J. Histochem. Cytochem. 49: 71-79 (2002); Hirai, T. et al., Circulation 79: 791-96 (1989); Ejiri, M. et al., J. Cardiology 20: 31-37 (1990). Because myocardial cells have virtually no ability to regenerate, myocardial infarction leads to permanent cardiac dysfunction due to contractile-muscle cell loss and replacement with nonfunctioning fibrotic scarring. Frangogiannis, N. G., et al., Cardiovascular Res. 53(1): 31-47 (2002). Moreover, compensatory hypertrophy of viable cardiac muscle leads to microvascular insufficiency that results in further demise in cardiac function by causing myocardial muscle hibernation and apoptosis of hypertrophied myocytes in the peri-infarct zone.

Among survivors of myocardial infarction, residual cardiac function is influenced most by the extent of ventricular remodeling (meaning changes in size, shape, and function, typically a decline in function, of the heart after injury). Alterations in ventricular topography (meaning the shape, configuration, or morphology of a ventricle) occur in both infarcted and healthy cardiac tissue after myocardial infarction. Pfeffer, M. A., Braunwald, E., Circulation 81: 1161-72 (1990). Ventricular dilatation (meaning a stretching, enlarging or spreading out of the ventricle that shifts ventricular shape from ellipsoid to spheroid) causes a decrease in global cardiac function and is affected most by the infarct size, infarct healing and ventricular wall stresses. Recent efforts to minimize remodeling have been successful by limiting infarct size through rapid reperfusion (meaning restoration of blood flow) using thromobolytic agents and mechanical interventions, including, but not limited to, placement of a stent, along with reducing ventricular wall stresses by judicious use of pre-load therapies and proper after-load management. Id. Without being limited by theory, neurohormonal pathways may mediate pre-load and after-load effects. For example, beta-blockers can act as antagonists of the sympathetic nervous system; and angiotensin-converting enzyme (ACE) inhibitors, angiotensin II receptor blockers (ARBs), and aldosterone inhibitors can act as antagonists of renin-angiotensin-aldosterone system. Regardless of these interventions, a substantial percentage of patients experience clinically relevant and long-term cardiac dysfunction after myocardial infarction. Sheiban, I. et al., J. Am. Coll. Cardiol. 38: 464-71 (2001). Despite revascularization of the infarct related artery circulation and appropriate medical management to minimize ventricular wall stresses, a significant percentage of patients experience ventricular remodeling, permanent cardiac dysfunction, and consequently remain at an increased lifetime risk of experiencing adverse cardiac events, including death. Paul et al., Am. Heart J. 131: 710-15 (1996); Pfeffer, M. A., Braunwald, E., Circulation 81: 1161-72 (1990).

At the cellular level, immediately following a myocardial infarction, transient generalized cardiac dysfunction uniformly occurs. In the setting of a brief (i.e., lasting three minutes to five minutes) coronary artery occlusion, energy metabolism is impaired, leading to demonstrable cardiac muscle dysfunction that can persist for up to 48 hours despite immediate reperfusion. This so-called “stunned myocardium phenomenon” occurs subsequent to or after reperfusion and is thought to be a result of reactive oxygen species. The process is transient and is not associated with an inflammatory response. Frangogiannis, N. G., et al., Cardiovascular Res. 53(1): 31-47 (2002). After successful revascularization, significant recovery from stunning occurs within three to four days, although complete recovery may take much longer. Boli, R., Prog. Cardiovascular Disease 40(6): 477-515 (1998); Sakata, K. et al., Ann. Nucleic Med. 8: 153-57 (1994); Wollert, K. C. et al., Lancet 364: 141-48 (2004).

Coronary artery occlusion of more significant duration, i.e., lasting more than five minutes, leads to myocardial ischemia (i.e., an insufficient blood flow to the heart's muscle mass) and is associated with a significant inflammatory response that begins immediately after reperfusion and can last for up to several weeks. Frangogiannis, N. G., et al., Cardiovascular Res. 53(1): 31-47 (2002); Frangogiannis, N. G. et al., Circulation 98: 687-798 (1998).

The inflammatory process following reperfusion is complex. Initially it contributes to myocardial damage but later leads to healing and scar formation. This complex process appears to occur in two phases. In the first so-called “hot” phase (within the first five days), reactive oxygen species (in the ischemic myocardial tissue) and complement activation generate a signal chemotactic for leukocytes (chemotaxis is the directed motion of a motile cell, organism or part towards environmental conditions it deems attractive and/or away from surroundings it finds repellent) and initiate a cytokine cascade. Lefer, D. J., Granger, D. N., Am. J. Med. 4:315-23 (2000); Frangogiannis, N. G., et al., Circulation 7:699-710 (1998); Sackner-Bernstein, J. D., Curr. Cardiol. Rep. 2(2):112-119 (2000). Mast cell degranulation, tumor necrosis factor alpha (TNF-α) release, and increased interleukin-6 (IL-6), intercellular adhesion molecule 1 (“ICAM-1” or CD-54, a receptor typically expressed on endothelial cells and cells of the immune system), selectin (L, E and P) and integrin (CD 11a, CD11b and CD18) expression all appear to contribute to neutrophil accumulation and degranulation in ischemic myocardium. Frangogiannis, N. G. et al., Circulation 7: 699-710 (1998), Kurrelmeyer, K. M, et al., Proc. Nat'l Acad. Sci. 10: 5456-61 (2000); Lasky, L. A., Science 258: 964-69 (1992); Ma, X. L., et al., Circulation 88(2): 649-58 (1993); Simpson, P. J. et al., J. Clin. Invest. 2: 624-29 (1998). Neutrophils contribute significantly to myocardial cell damage and death through microvascular obstruction and activation of neutrophil respiratory burst pathways after ligand-specific adhesion to cardiac myocytes. Entman, M. L., et al., J. Clin. Invest. 4: 1335-45 (1992). During the “hot” phase, angiogenesis is inhibited due to the release of angiostatic substances, including interferon gamma-inducible protein (IP 10). Frangogiannis, N. G., et al., FASEB J. 15: 1428-30 (2001).

In the second phase, the cardiac repair process begins (about day 6 to about day 14), which eventually leads to scar formation (about day 14 to about day 21) and subsequent ventricular remodeling (about day 21 to about day 90). Soon after reperfusion, monocytes infiltrate the infarcted myocardium. Attracted by complement (C5a), transforming growth factor β1 (“TGF-β1”) and monocyte chemotactic protein 1 (“MCP-1”), monocytes differentiate into macrophages that initiate the healing process by scavenging dead tissue, regulating extracellular matrix metabolism, and inducing fibroblast proliferation. Birdshall, H. H., et al., Circulation 3: 684-92 (1997). Secretion of interleukin 10 (IL-10) by infiltrating lymphocytes also promotes healing by down-regulating inflammatory cytokines and influencing tissue remodeling. Frangogiannis, N. G. et al., J. Immunol. 5:2798-2808 (2000). Mast cells also appear to be involved in the later stages of myocardial repair by participating in the formation of fibrotic scar tissue. Stem Cell Factor (SCF) is a potent attractor of mast cells. SCF mRNA has been shown to be up-regulated in ischemic myocardial segments in a canine model of myocardial infarction and thus may contribute to mast cell accumulation at ischemic myocardial sites. Frangogiannis, N. G. et al., Circulation 98: 687-798 (1998). Mast cell products (including TGF-B, basic fibroblast growth factor (bFGF), vascular endothelial growth factor (VEGF) and gelatinases A and B) induce fibroblast proliferation, influence extracellular matrix metabolism, and induce angiogenesis. Fang, K. C., et al., J. Immunol. 162: 5528-35 (1999); Takeshi, S., et al., Cardiology 93: 168-74 (2000).

Following a myocardial infarction, neoangiogenesis occurs after the “hot” phase of the inflammatory process subsides (about day 5) coincident with rising levels of VEGF (VEGF peaks at about day 7 and gradually subsides to baseline at about day 14 to about day 21). During this phase of the healing process, endothelial precursor cells (EPCs) are mobilized and recruited to the infarct site. Shinitani, S., et al., Circulation 103: 2776-79 (2001). Without being limited by theory, it has been suggested that the chemokine stromal cell derived factor-1 (SDF-1), which is the ligand for the CXCR-4 chemokine receptor expressed by CD34+ cells, also plays a role in homing of cells to areas of ischemic damage. Ceredini, D. J., et al., Nature Medicine 10: 858-63 (2004); Askari, A., et al., Lancet 362: 697-703 (2003); Yamaguchi, J. et al., Circulation 107: 1322-34 (2003). While it is known that SDF-1 plays a role in hematopoiesis and is involved in migration, homing and survival of hematopoietic progenitors, and while SDF-1 has been implicated in ischemic neovascularization in vivo by augmenting EPC recruitment to ischemic sites (Yamaguchi et al. Circulation 107:1322-1328 (2003), SDF-1's role in neoangiogenesis is not certain. There is suggestive evidence implicating SDF-1. For example, SDF-1 gene expression is upregulated during hypoxia, a deficiency of oxygen in the tissues, by hypoxia inducible factor-1. Furthermore, CD34+ cells are capable of homing to areas of ischemia, rich in SDF-1, including infarcted myocardium. Askari et al., Lancet 362: 697-703 (2003). Moreover, virtually all CD34+ CXCR-4+ cells co-express VEGF-2 and therefore migrate in response to VEGF as well as SDF-1. Peichev M., et al., Blood 95: 952-58 (2000). CD34+CXCR-4+VEGF-1 cells, once recruited, are capable of contributing to neoangiogenesis. Yamaguchi, J. et al., Circulation 107: 1322-34 (2003). Overexpression of SDF-1 expression by delivery of plasmid-DNA encoding human SDF-1 into infarct border zones in rats with ischemic heart failure was shown to promote angiogenesis and improved cardiac function; there also was evidence of scar remodeling with a trend toward decreased myocardial fibrosis. (Sundararaman, S. et al., Gene Therapy, 18: 867-873 (2011)).

In the acute phase of cerebral infarction, accumulating data suggest that free radicals including superoxide, hydroxy radical and nitric oxide are one of the most important factors to cause brain damage. For example, nitrotyrosine (a marker of endogenous production of peroxynitrite, which is readily produced from superoxide and nitric oxide) were detected in neurons and intraparenchymal vascular walls during post-ischemic reperfusion (Kotaro, T et al., Clinical Neurology, 41(12): 1052-1054, 2001). The acute phase response after cerebral infarction is less common and weaker than that reported after myocardial infarction. (Syrjanen, J. et al., “Acute phase response in cerebral infarction,” J. Clin. Pathol., 42: 63-68 (1989)).

### 10. Chemokines

Chemokines (Chemotactic Cytokines) Constitute a Family Of Low Molecular Mass (8-11 kDa) structurally-related proteins with diverse immune and neural functions (Mackay, C., Nat Immunol 2: 95-101, 2001; Youn, B. et al., Immunol Rev, 177: 150-174, 2000) that can be categorized into four subfamilies (C, CC, CXC and CX3C) based on the relative positions of conserved cysteine residues (Rossi, D. et al., Annu Rev Immunol, 18: 217-242, 2000). Chemokines affect cells by activating surface receptors that are seven-transmembrane-domain G-protein-coupled receptors; leukocyte responses to particular chemokines are determined by their complement of chemokine receptors. The binding of the chemokine to the receptor activates signaling cascades that culminate in the rearrangement, change of shape, and cell movement of actin.

Stromal cell-derived factor-1 (SDF-1, CXCL12) is a member of the CXC chemokine subfamily and the only known ligand for the G protein-coupled receptor CXCR-4 (Rossi, D. et al., Ann Rev Immunol, 18: 217-242, 2000). Two isoforms of SDF-1 (SDF-1α and SDF-1β), which only differ by four amino acids at the C-terminus, are generated from a single gene by differential RNA splicing. The primary structure of SDF-1 is highly conserved across species, with only one amino acid difference between the human and mouse proteins (Shirozu, M. et al., Genomics, 28: 495-500, 1995).

CXCR-4 and SDF-1 are highly expressed during development in the cerebellum, hippocampus and neocortex and constitutively expressed in the brain during adulthood (Stumm, R. et al., J. Neurosci, 23:5123-5130, 2003). Moreover, CXCR-4 and SDF-1 are expressed in neurons and glia in the adult brain (Felszeghy, K, Neuroimmunomodulation, 11: 404-413, 2004).

In addition, recent studies have suggested that SDF-1 and CXCR-4 play an important role in CNS homeostasis (Peng, H et al., J Neurosci Res, 76:35-50, 2004). For example, mice with targeted mutations in CXCR-4 or SDF-1 showed defective migration of cerebellar granule cells (Zou, Y. et al., Nature 393:595-599, 1998; Ma, Q. et al. Proc Natl Acad Sci USA, 95:9448-9453, 1998) or marked alteration of the morphology of the dentate gyrus (DG) (Lu, M. et al., Proc Natl Acad Sci USA, 99: 7090-7095, 2002; Bagri, A. et al., Development 129: 4249-4260, 2002).

Neural stem cells (NSCs) in the subventricular zone (SVZ) and subgranular zone (SGZ) in the adult rat brain have been shown to express CXCR-4 (Ji, J. et al., Neurosci Lett, 355: 236-240, 2004; Tran, P. et al., J Neurosci Res, 76: 20-34, 2004). In addition, it was shown that NSCs in the SGZ migrate to the dentate gyrus (DG), which synthesizes SDF-1, and differentiate into neurons, thereby contributing to neurogenesis and neuroplasticity (Bagri, A. et al., Development 129: 4249-4260, 2002). Meanwhile, studies also have found that nestin-positive, cultured NSCs isolated from the rat SVZ express CXCR-4 and migrate toward SDF-1 (Pujol, F. et al., J Cell Sci, 118: 1071-1080, 2005; Dziembowska, M. et al., Glia, 50: 258-269, 2005). These studies suggest that SDF-1/CXCR-4 expression after TBI may contribute to neural stem cell migration and neurogenesis (Peng, H. et al., J Neurosci Res, 76: 35-50, 2004; Imitola, J. et al., Proc Natl Acad Sci USA, 101:18117-18122, 2004).

### 11. Chemokine Signaling in Neural Injury

In the developing brain, neurons and glia express various chemokine receptors and are therefore potential targets for chemokine signaling. For example, previous studies have shown that chemokines, in particular SDF-1 and its receptor CXC chemokine receptor 4 (CXCR-4), regulate neonatal oligodendrocyte maturation and myelination, axonal growth, neuronal proliferation and neuronal survival (Tysseling, V. et al., J. Neuroinflammation, 8:16, 2011). In mice, targeted deletion of CXCR-4, or its ligand CXCL12, causes perinatal death, indicating that this ligand and its receptor have a vital developmental function (Charo, I. et al., N Engl J Med, 354:610-621, 2006).

Neural injury results in a specific cascade of biologic events in the surrounding region. This pathophysiology includes an acute inflammatory response with surrounding edema, causing disturbed nutritive blood flow, tissue ischemia, and eventually, apoptosis. Neurotrauma is associated with short term release of SDF-1 between days 2 and 11 (Deng J et al, Neurological Science 2011, 32:641-51). Previous studies found that, within the first 1-2 weeks, surrounding tissue with high SDF-1 levels attracts stem cells (Lu D. et al., Regeneration and Transplant, 12(3):559-563, 2011); and that, in rodent models of neurotrauma, SDF-1 release is associated with better neurologic outcome (Lu, Regen and Transplant 2001, 12:559. Li, Brain Research 2012, 1444:76). These data indicate that neither expression nor translation of SDF-1 are necessarily upregulated, but rather that increased release from constitutively produced SDF-1 is sufficient (Itoh T et al, Neurological Research 2009, 31:90-102).

Models of spinal neurotrauma appear to show similar physiology (Tysseling V M et al, Journal of Neuroinflammation 2011, 8:16). For example, previous studies have shown that SDF-1 and its receptor CXCR-4 are expressed in the normal, uninjured spinal cord, and that following the breach in blood brain barrier after severe spinal cord injury, multiple sources of both proteins contribute to specific post-injury signaling. It was shown that CXCR-4-expressing macrophages migrate through the spinal cord toward the sources of SDF-1 and arrive in the peripheral spinal cord, toward the SDF-1 in the meninges, and toward the intact dorsal corticospinal tract (dCST). It was reported that this migration occurs between 1 and 2 weeks post injury; and that CXCR-4-expressing ependymal cells remain post injury, but are in fewer number than in the uninjured spinal cord.

Studies of neurotrauma to rodents reveal that focal destruction is associated with a penumbra of affected tissues. Itoh and colleagues (Itoh et al, Neurological Research, 2009, 31:90-102) detected high levels of SDF-1 leaking from cells into the interstitial space, even without up-regulation of its production, in a manner that reflects local tissue signaling for angiogenesis. In parallel, the model revealed that cells expressing CXCR-4 are attracted to this region. With a peak release of SDF-1 at 3 days that returns to baseline at the end of a week, this physiology appears ripe for cell therapy.

Using animal models of neurotrauma, previous studies have established: (1) that release of SDF-1 is coupled to increase in microvessel density (Li S et al, Brain Research 2012, 1444:76-86) similar to what is observed in the myocardium; and (2) that mesenchymal stem cells administered intravenously are attracted by an SDF-1 gradient (Lu D et al, Regeneration and Transplantation, 2001, 12(3):559-63).

### 12. Bone Marrow-Derived Hematopoietic Stem Cells

Bone marrow consists of a variety of precursor and mature cell types, including hematopoietic cells (the precursors of mature blood cells) and stromal cells (the precursors of a broad spectrum of connective tissue cells), both of which appear to be capable of differentiating into other cell types (Wang, J. S. et al., J. Thorac. Cardiovasc. Surg. 122: 699-705, 2001; Tomita, S. et al., Circulation 100 (Suppl. II): 247-256, 1999; Saito, T. et al., Tissue Eng. 1: 327-343, 1995). Unmodified (i.e., not fractionated) marrow or blood-derived cells have been used in several clinical studies (for example, Hamano, K. et al., Japan Cir. J. 65: 845-847, 2001; Strauer, B. E., et al., Circulation 106: 1913-1918, 2002; Assmus, et al., Circulation 106: 3009-3017, 2002; Dobert, N. et al., Eur. J. Nucl. Med. Mol. Imaging, 8:1146-1151, 2004; Wollert, K. C. et al., Lancet 364: 141-48, 2004). Since the mononuclear fraction of bone marrow contains stromal cells, hematopoietic precursors, and endothelial precursors, the relative contribution of each of these populations to the observed effects, if any, remains unknown.

CD34 is a hematopoietic stem cell antigen selectively expressed on hematopoietic stem and progenitor cells derived from human bone marrow, blood and fetal liver (Yin et al., Blood 90: 5002-5012 (1997); Miaglia, S. et al., Blood 90: 5013-21 (1997)). Cells that express CD34 are termed CD34+. Stromal cells do not express CD34 and are therefore termed CD34−. CD34+ cells represent approximately 1% of bone marrow derived nucleated cells; CD34 antigen also is expressed by immature endothelial cell precursors; mature endothelial cells do not express CD34+ (Peichev, M. et al., Blood 95: 952-958, 2000). In vitro, CD34+ cells derived from adult bone marrow give rise to a majority of the granulocyte/macrophage progenitor cells (CFU-GM), some colony-forming units-mixed (CFU-Mix) and a minor population of primitive erythroid progenitor cells (burst forming units, erythrocytes or BFU-E) (Yeh, et al., Circulation 108: 2070-2073, 2003). CD34+ cells also may have the potential to differentiate into or to contribute to the development of new neural cells (including microglia, astrocytes and neurons), albeit at low frequency (Asheuer, M. et al. Proc Natl Acad Sci, USA, 101(10): 3557-3562; Reali, C. et al., Exp Neurol. 197(2): 399-406, 2006; Sigurjonsson, O. et al., Proc Natl Acad Sci, USA, 102(14): 5227-5232).

Techniques have been developed using immunomagnetic bead separation to isolate a highly purified and viable population of CD34+ cells from bone narrow mononuclear cells. See U.S. Pat. Nos. 5,536,475, 5,035,994, 5,130,144, 4,965,205, the entire contents of each which are incorporated herein by reference in its entirety.

### 13. Cell Therapy for Treating Traumatic Brain or Spinal Cord Injury

Originally, it was thought that recovery from traumatic brain or spinal cord injuries was severely limited because: (i) the nervous system, unlike many other tissues, has a limited capacity for self-repair; (ii) mature nerve cells lack the ability to regenerate, and (iii) neural stem cells, although they exist even in the adult brain, have a limited ability to regenerate new functional neurons in response to injury. Recent studies, however, have indicated that the mammalian nervous system has the potential to replenish populations of damaged and/or destroyed neurons via the proliferation of neural stem cells. This has generated intense interest in the possibility of repairing the nervous system by transplanting cells that can replace those lost through damage or release, or preserve those cells that remain and their function. By preserving cells that remain in salvageable regions and their function, transplanted stem cells also may lead to regeneration of new nerve cells, for example, by neural stem cells. To date, however, no ideal therapy exists to do so in patients following traumatic brain or spinal cord injury, more particularly, in patients with chronic traumatic encephalopathy (CTE).

We have shown that after an acute myocardial infarction, a therapeutic amount of a composition comprising a nonexpanded, isolated population of autologous mononuclear cells containing at least 0.5×10⁶ potent SDF-1 mobile CD34+CXCR-4+ cells that have chemotactic activity improves an infarct area injury by prevention of cardiomyocyte loss after AMI through enhancement of perfusion and prevention of apoptosis. These benefits in an AMI patient are exerted through a paracrine and neoangiogenic effect, which affects immediate cell death and later changes consistent with ventricular remodeling (AMI) (Quyyumi, A. et al., Am Heart J 161(1): 98-105 (2011); U.S. Pat. No. 7,794,705, U.S. Pat. No. 8,088,770, U.S. Published Application 2010-0143317, U.S. Published Application 2011-0076255, and U.S. Published Application 2012-0071855, each of which is incorporated by reference in its entirety).

The present invention relates to a composition comprising a chemotactic hematopoietic stem cell product containing a nonexpanded isolated population of autologous mononuclear cells enriched for CD34+ cells, which further contains a subpopulation of potent SDF-1 mobile, CD34+/CXCR-4+ cells that have CXCR-4 mediated chemotactic activity and are functionally VEGFR-2—that can migrate to the injured parenchyma in the brain or spinal cord based on an SDF-1 gradient, and by a similar mechanism, preserve neuronal cells that remain and their function.

### SUMMARY OF THE INVENTION

According to one aspect, the described invention provides a method for treating a vascular insufficiency of a cerebral artery, a spinal cord artery, or a branch thereof following a traumatic injury to head or spine that results in an injury to paranchyma of brain, spinal cord, or both, the method comprising: (a) administering to a subject in need thereof via a delivery device a therapeutic amount of a pharmaceutical composition comprising:

(i) a therapeutic amount of a sterile chemotactic hematopoietic stem cell product containing an isolated, nonexpanded population of autologous mononuclear cells comprising a subpopulation of CD34+ cells;

wherein the pharmaceutical composition is
formulated for administration parenterally;
characterized in that the isolated population of mononuclear cells comprising a subpopulation of CD34+ cells further contains a subpopulation of potent SDF-1 mobile CD34+/CXCR-4+ cells that have CXCR-4-mediated chemotactic activity, such that the therapeutic amount comprises at least 0.5×10⁶ potent SDF-1-mobile CD34+CXCR-4+ cells that have CXCR-4-medicated chemotactic activity; and further characterized as having the following properties for at least 24 hours following acquisition of the chemotactic hematopoietic stem cell product when tested in vitro after passage through a catheter:
(1) at least 70% of the cells are CD34+ cells;
(2) retains at least 2% of the CXCR-4-mediated chemotactic activity of the subpopulation of subpopulation of potent SDF-1 mobile CD34+/CXCR-4+ cells that have CXCR-4-mediated chemotactic activity measured prior to purification;
(3) is at least 70% viable; and
(4) is able to form hematopoietic colonies in vitro; and

(ii) a stabilizing amount of serum, which is effective to retain the CXCR-4-mediated chemotactic activity and hematopoietic colony forming activity of the population of SDF-1 mobile CD34+CXCR-4+ cells from acquisition to infusion; and

(b) monitoring the subject's cognitive and neurologic functions,

wherein the therapeutic amount of the sterile chemotactic hematopoietic stem cell product is effective to improve perfusion and to preserve existing nerve cells and their function in an area of ischemia in paranchyma of the injured brain, spinal cord, or both.

According to one embodiment of the method, the traumatic injury is a severe traumatic brain injury that requires craniectomy. According to another embodiment, the severe traumatic brain injury comprises acute subdural hematoma or parenchymal hematoma. According to another embodiment, the traumatic injury is a mild to moderate closed head injury that does not require craniectomy. According to another embodiment, the mid to moderate closed head injury is selected from the group consisting of cerebral concussion, cerebral contusion, epidural hematoma, subdural hematoma, intraventricular hemorrhage, and diffuse axonal injury. According to another embodiment, the injury to brain, spinal cord or both comprises an infarct area injury selected from the group consisting of apoptotic nerve cell loss in the infarct area; adverse remodeling after an acute cerebral infarction, when compared to controls; a progressive decline in cognitive function following the acute cerebral infarction; hypoperfusion of at least one ischemic peri-infarct zone; and a combination thereof. According to another embodiment, the method is capable of improving microvascular blood flow in the infarct area, of decreasing area of the infarct injury, of decreasing infarct mass, of increasing perfusion of at least one ischemic peri-infarct zone of nerve tissue, or a combination thereof when compared to controls. According to another embodiment, the chemotactic hematopoietic stem cell product is administered after peak inflammatory cytokine cascade production in an infarcted area and before completion of scar formation in the infarcted area. According to another embodiment, the injury to the brain, spinal cord, or both places the subject at risk for developing chronic traumatic encephalopathy (CTE) of the brain, spinal cord, or both. According to another embodiment, the chronic traumatic encephalopathy (CTE) is associated with progressive tauopathy in the injured brain, spinal cord or both. According to another embodiment, the pharmaceutical composition further comprises a therapeutic amount of at least one compatible therapeutic agent. According to another embodiment, the therapeutic amount of the compatible therapeutic agent is capable of promoting function of existing nerve cells to compensate for loss of function due to neuronal death, of regenerating new nerve cells, or both. According to another embodiment, the compatible therapeutic agent comprises a cytokine, a vasoactive agent, an anticoagulant agent, an antiplatelet agent, an antihypercholesterolemic agent, or a combination thereof. According to another embodiment, the anti-coagulant agent is selected from the group consisting of a coumarin, heparin, an inhibitor of Factor Xa, batroxobin, hementin, and a combination thereof. According to another embodiment, the delivery device is coated with the anticoagulant agent. According to another embodiment, the compatible therapeutic agent comprises a cytokine, a placental growth factor, granulocyte colony-stimulating factor, macrophage colony-stimulating factor, a vascular endothelial growth factor, neuregulin-1, tumor necrosis factor-like weak inducer of apoptosis, or a combination thereof. According to another embodiment, the cytokine is at least one selected from the group consisting of vascular endothelial growth factor (VEGF), placental growth factor (PIGF), granulocyte colony-stimulating factor (G-CSF), and macrophage colony-stimulating factor (M-C SF). According to another embodiment, the vascular endothelial growth factor is selected from the group consisting of VEGF-A, VEGF-B, VEGF-C, and VEGF-D. According to another embodiment, the at least one compatible therapeutic agent is placental growth factor. According to another embodiment, the administering parenterally is by direct injection or by infusion into the paranchyma of the injured brain, spinal cord, or both. According to another embodiment, the therapeutic amount of the chemotactic hematopoietic stem cell product is administered via stereotactically-guided direct injection. According to another embodiment, the therapeutic amount of the chemotactic hematopoietic stem cell product is administered via impedance-guided direct injection. According to another embodiment, the administering parenterally is performed intravascularly. According to another embodiment, the pharmaceutical composition is infused into an artery or a branch thereof. According to another embodiment, the administering parenterally is performed at one or more infusion dates. According to another embodiment, the artery is a carotid artery or a branch thereof. According to another embodiment, the artery is a cerebral artery or a branch thereof. According to another embodiment, the delivery device is a catheter. According to another embodiment, the catheter comprises an anticoagulant agent. According to another embodiment, the catheter has an internal diameter of at least 0.36 mm. According to another embodiment, the stabilizing amount of serum is from about 0.1% to about 70% (v/v).

### BRIEF DESCRIPTION OF THE DRAWINGS

FIG. 1 shows an illustrative lateral view of the human brain (Stedman's Medical Dictionary, 27^{th} Edition, plate 7 at A7 (2000)).

FIG. 2 shows an illustrative view of the circle of Willis and principal arteries of the brain (Correlative Neuroanatomy & Functional Neurology, 18^{th} Ed., p. 48 (1982)).

FIG. 3 shows an illustrative view of the arterial supply of the cerebral cortex. 1: orbitofrontal artery; 2: prerolandic artery; 3: rolandic artery; 4: anterior parietal artery; 5: posterior parietal artery; 6: angular artery; 7: posterior temporal artery; 8: anterior temporal artery; 9: orbital artery; 10: frontopolar artery; 11: callosomarginal artery; 12: posterior internal frontal artery; 13: pericallosal artery. (Correlative Neuroanatomy & Functional Neurology, 18^{th} Ed., p. 50 (1982)).

FIG. 4 shows an illustrative view of the cerebral arteries.

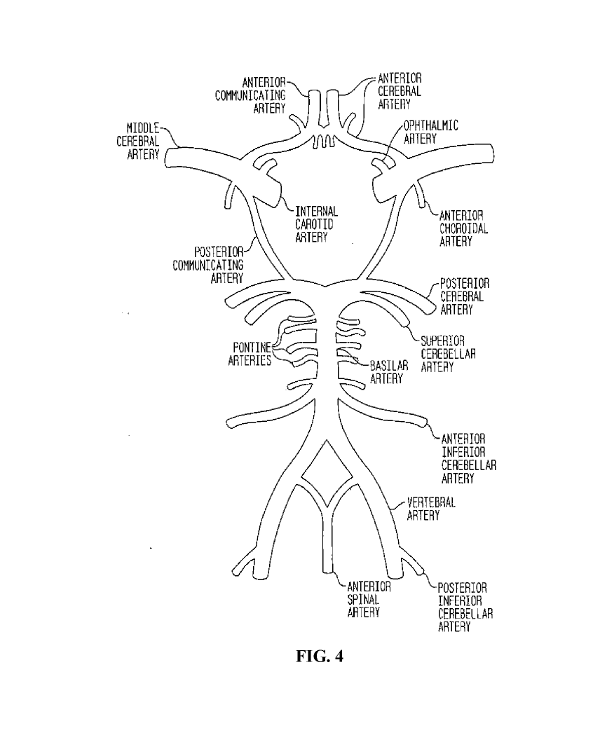

FIG. 5 shows an illustrative view of the cerebral arteries. (from Netter FH. The CIBA Collection of Medical Illustrations: Volumes 1, Nervous System. Vol. 1. Part I. CIBA: USA. 1986. pp. 256).

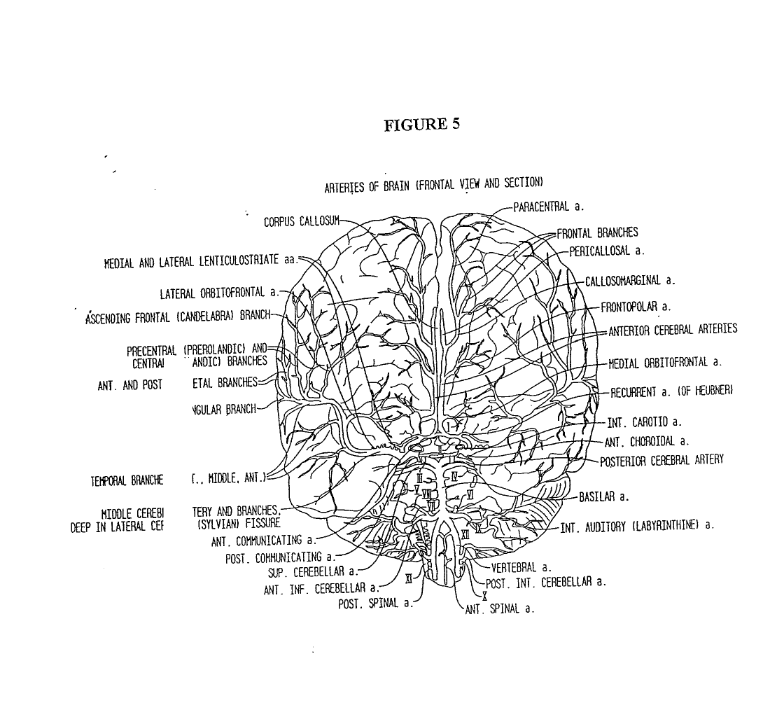

### DETAILED DESCRIPTION OF THE INVENTION

### Glossary

The following definitions set forth the parameters of the present invention.

The term “administer” as used herein in its various grammatical forms means to give or to apply. The term “administering” as used herein includes in vivo administration, as well as administration directly to tissue ex vivo. Generally, compositions may be administered systemically either parenterally or topically in dosage unit formulations containing conventional nontoxic pharmaceutically acceptable carriers, adjuvants, and vehicles as desired, or may be locally administered by means such as, but not limited to, injection, implantation, grafting, topical application, or parenterally. A means of administering cells may include, but is not limited to, infusion.

As used herein, the term “angiogenesis” refers to sprouting of new blood vessels from pre-existing blood vessels.

The term “anticoagulant agent” as used herein refers to any agent or agents capable of preventing or delaying blood clot formation in vitro and/or in vivo. Examples of anticoagulant include, but are not limited to, a coumarin (vitamin K antagonists, e.g., warfarin, acenocoumarol, phenprocoumon, atromentin, brodifacoum, phenindione), heparin (including a low molecular weight heparin), a synthetic pentasaccharide inhibitor of Factor Xa (e.g., fondaparinux, idraparinux), a direct Factor Xa inhibitor (e.g., rivaroxaban and apixaban), a direct thrombin inhibitor (e.g., hirudin, lepirudin, bivalirudin, argatroban, and dabigatran), batroxobin (a toxin from a snake venom), and hementin (an anticoagulant protease form the salivary glands of Haementeria ghilianii).

The term “antihypercholesterolemic agent” as used herein refers to an agent that reduce and control the level of cholesterol for example in the blood and liver. Examples of antihypercholesterolemic agents include, but are not limited to, atorvastatin, cholestyramine, gemfibrozil, lovastatin, simvastatin, sitosterol, pravachol, pravastatin, etc.

The term “antiplatelet agent” as used herein refers to an agent or agents capable of decreasing platelet aggregation and inhibit thrombus formation. Examples of antiplatelet agents include, but are not limited to, cyclooxygenase (COX) inhibitors (e.g. aspirin), adenosine diphosphate (ADP) receptor inhibitors (e.g. thionepyridines, such as clopidogrel, prasugrel, ticlopidine), phosphodiesterase inhibitors (e.g. cilostazol, dipyridamole), glycoprotein IIb/IIIb inhibitors (e.g. tirofiban, eptifibatide, abciximab), etc.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways.

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligomerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon agregation and mitochondrial localization, induces cytochrome c release. Akt is a critical regulator of GSK-3, and phosphorylation and inactivation of GSK-3 may mediate some of the antiapoptotic effects of Akt.

The term “carotid artery” as used herein refers to a major artery that delivers blood through the neck to the brain. There is one carotid artery on each side of the neck.

The term “catheter” as used herein refers to any hollow instrument capable of penetrating body tissue or interstitial cavities and providing a conduit for selectively injecting a solution or gas. The term “balloon catheter” as used herein refers to a type of “soft” thin flexible tube having an inflatable “balloon” at its tip which is used during some catheterization procedures to enlarge a narrow opening or passage within the body. The term “catheterization” as used herein refers to a procedure in which a catheter is passed into the body.

The term “cerebral artery” as used herein refers to any of the arteries supplying blood to the cerebral cortex of the brain and their branches.

The term “c-kit” refers to a protein on the surface of some cells that binds to stem cell factor (a substance that causes certain types of cells to grow). Altered forms of this receptor may be associated with some types of cancer.

The term “CD34+ cells” as used herein refers to hematopoietic stem and progenitor cells derived from human bone marrow that “are positive for” i.e., “express”, a hematopoietic stem cell antigen, at least a subpopulation of which express CXCR-4, and that can migrate to areas of injury.

The term “CD38” refers to a protein marker present on macrophages, dendritic cells, and activated B and NK cells, which may mediate the adhesion between lymphocytes and endothelial cells.

The terms “CD45” and “common leukocyte antigen” refer to a protein tyrosine phosphatase (PTP) located in hematopoietic cells except erythrocytes and platelets.

The term “CD59” refers to a glycosylphosphatidylinositol (GPI)-linked membrane glycoprotein which protects human cells from complement-mediated lysis.

The term “chemotaxis” refers to the directed motion of a motile cell or part towards environmental conditions it deems attractive and/or away from surroundings it finds repellent.

The term “Circle of Willis” as use herein refers to a complete ring of arteries at the base of the brain that is formed by the cerebral and communicating arteries.

The terms “composition” and “formulation” are used interchangeably herein to refer to a product of the present invention that comprises all active and inert ingredients. The term “active” refers to the ingredient, component or constituent of the compositions of the present invention responsible for the intended therapeutic effect. The terms “pharmaceutical formulation” or “pharmaceutical composition” as used herein refer to a formulation or composition that is employed to prevent, reduce in intensity, cure or otherwise treat a target condition or disease.

The term “CXCR-4” as used herein refers to a G-protein-linked chemokine receptor.

The term “craniectomy” as used herein refers to the opening of the skull and removal of a portion of it.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNF and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines

The term “colony stimulating factor” refers to a cytokine responsible for controlling the production of white blood cells. Types include granulocyte colony stimulating factor (G-CSF), macrophage colony stimulating factor (M-CSF), and granulocyte macrophage colony stimulating factor (GM-CSF).

The term “hematopoietic stem cell” refers to a cell isolated from the blood or from the bone marrow that can renew itself, differentiate to a variety of specialized cells, mobilize out of the bone marrow into the circulating blood, and can undergo programmed cell death (apoptosis). According to some embodiments of the present invention, hematopoietic stem cells derived from human subjects express at least one type of cell surface marker, including, but not limited to, CD34, CD38, HLA-DR, c-kit, CD59, Sca-1, Thy-1, and/or CXCR-4, or a combination thereof.

The term “HLA-DR” refers to a human class II histocompatibility antigen present on several cell types, including antigen-presenting cells, B cells, monocytes, macrophages, and activated T cells.

The term “infarction” as used herein refers to a sudden insufficiency of arterial or venous blood supply due to, for example, emboli, thrombi, mechanical factors, or pressure that produces a macroscopic area of necrosis. The term “infarct” as used herein refers to the area of necrosis resulting from a sudden insufficiency of arterial or venous blood supply. The term “cerebral infarction” as used herein refers to a loss of brain tissue subsequent to the transient or permanent loss of circulation and/or oxygen delivery to the cerebrum.

The term “interleukin” as used herein refers to a cytokine secreted by white blood cells as a means of communication with other white blood cells.

The term “ischemia” as used herein refers to a lack of blood supply and oxygen due to mechanical obstruction (mainly arterial narrowing or disruption) of the blood supply. Ischemia occurs when reduced perfusion pressure distal to an abnormal narrowing (stenosis) of a blood vessel is not compensated by autoregulatory dilation of the resistance vessels.

The terms “VEGF” or “vascular endothelial growth factor” are used interchangeably to refer to a family of cytokines that mediates numerous functions of endothelial cells including proliferation, migration, invasion, survival, and permeability. VEGF is critical for angiogenesis. In mammals, five VEGF ligands, which occur in several different splice variants and processed forms, have been identified so far. These ligands bind in an overlapping pattern to three receptor tyrosine kinases (RTKs), known as VEGF receptor-1, -2 and -3 (VEGFR1-3), as well as to co-receptors (here defined as VEGF-binding molecules that lack established VEGF-induced catalytic function), such as heparan sulphate proteoglycans (HSPGs) and neuropilins. (Olsson, et al, Nature Revs./Molec. Cell Biol. 7: 359- (2006), which is incorporated by reference herein in its entirety).

The term “chemokine” as used herein refers to a class of chemotactic cytokines that signal leukocytes to move in a specific direction. The term “chemotactic” refers to movement or orientation of a cell along a chemical concentration gradient either toward or away from a chemical stimulus.

The term “complete blood count” (CBC) refers to a laboratory test that provides detailed information about the amount and the quality of each of the blood cells types. It usually includes a measurement of each of the three major blood cells (red blood cells, white blood cells, and platelets) and a measure of the hemoglobin and hematocrit. “Hemoglobin” (HGB) refers to the number of grams of hemoglobin in a deciliter of blood (g/dL). Normal hemoglobin levels in healthy adult human subjects are about 14 g/dL to about 18 g/dL for men and about 12 g/dL to about 16 g/dL for women. As a rough guideline, hemoglobin generally should be about one-third the hematocrit. “Red Blood Cell Count” (RBC) refers to the total number of red blood cells in a quantity of blood. Normal ranges in human subjects are about 4.5 million cells/mm3 to about 6.0 million cells/mm3 for men and about 4.0 million cells/mm3 to about 5.5 million cells/mm3 for women. “White Blood Cell Count” (WBC) refers to the total number of white blood cells or leukocytes in a quantity of blood. Normal ranges in human subjects are about 4.3×103 cells/mm3 to about 10.8×103 cells/mm3. “Hematocrit” (HCT) refers to the proportion of red blood cells as a percentage of total blood volume. A normal hematocrit for human subjects is about 40% to about 55% for men and about 35% to about 45% for women.

The term “disease” or “disorder”, as used herein, refers to an impairment of health or a condition of abnormal functioning. The term “syndrome,” as used herein, refers to a pattern of symptoms indicative of some disease or condition. The term “condition”, as used herein, refers to a variety of health states and is meant to include disorders or diseases caused by any underlying mechanism or disorder, injury, and the promotion of healthy tissues and organs.

The term “hydrocephalus” as used herein refers to a condition marked by an excessive accumulation of cerebrospinal fluid resulting in dilation of the cerebral ventricles and raised intracranial pressure. Hydrocephalus may also result in enlargement of the cranium and atrophy of the brain.

As used herein, the term “inflammation” refers to a response to infection and injury in which cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue.

Regardless of the initiating agent, the physiologic changes accompanying acute inflammation encompass four main features: (1) vasodilation, which results in a net increase in blood flow, is one of the earliest s physical responses to acute tissue injury; (2) in response to inflammatory stimuli, endothelial cells lining the venules contract, widening the intracellular junctions to produce gaps, leading to increased vascular permeability which permits leakage of plasma proteins and blood cells out of blood vessels; (3) inflammation often is characterized by a strong infiltration of leukocytes at the site of inflammation, particularly neutrophils (polymorphonuclear cells). These cells promote tissue damage by releasing toxic substances at the vascular wall or in uninjured tissue; and (4) fever, produced by pyrogens released from leukocytes in response to specific stimuli.

During the inflammatory process, soluble inflammatory mediators of the inflammatory response work together with cellular components in a systemic fashion in the attempt to contain and eliminate the agents causing physical distress. The terms “inflammatory” or immuno-inflammatory” as used herein with respect to mediators refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1, interleukin-4, interleukin-6, interleukin-8, tumor necrosis factor (TNF), interferon-gamma, and interleukin 12.

The term “in-date” refers to the time interval between completion of acquiring from the subject a preparation comprising an enriched population of potent CD34+ cells from a subject under sterile conditions and initiating sterilely purifying potent CD34+ cells from the preparation. The term “out-date” refers to the time interval between completion of acquiring from the subject a preparation comprising an enriched population of potent CD34+ cells from a subject under sterile conditions and infusing the formulated pharmaceutical composition comprising a chemotactic hematopoietic cell product into the subject.

The terms “infuse” or “infusion” as used herein refer to the introduction of a fluid other than blood into a blood vessel of a subject, including humans, for therapeutic purposes.

The “infusion solution” of the present invention without autologous serum contains phosphate buffered saline (PBS) and 1% human serum albumin (HSA). According to some embodiments, the infusion solution is supplemented with 25 USP units/ml of heparin. According to some embodiments, the infusion solution is supplemented with serum. According to some embodiments, the serum is autologous.

The term “injury” refers to damage or harm caused to the structure or function of the body of a subject caused by an agent or force, which may be physical or chemical. The term “traumatic injury” as used herein refers to traumatic blows and trauma to the head or spine that causes damage or harm to the brain or connecting spinal cord, often without penetrating the skull or spinal column. In a traumatic brain injury, the initial trauma can result in an expanding hematoma, subarachnoid hemorrhage, cerebral edema, raised intracranial pressure (ICP), and cerebral hypoxia, which can, in turn, lead to severe secondary events due to low cerebral blood flow (CBF). The term “traumatic spinal cord injury” as used herein refers to a condition occurring when a traumatic event damages cells within the spinal cord, or when the nerve tracts that relay signals are severed or otherwise injured. Some of the most common types of spinal cord injury include contusion and compression.

The term “peri-infarct zone” as used herein refers to ischemic, but viable tissue that separates the central zone of progressive necrosis from surrounding normal neural tissues.

The term “macrophage” as used herein refers to a mononuclear, actively phagocytic cell arising from monocytic stem cells in the bone marrow. These cells are widely distributed in the body and vary in morphology and motility. Phagocytic activity is typically mediated by serum recognition factors, including certain immunoglobulins and components of the complement system, but also may be nonspecific. Macrophages also are involved in both the production of antibodies and in cell-mediated immune responses, particularly in presenting antigens to lymphocytes. They secrete a variety of immunoregulatory molecules.

The terms “microbe” or “microorganism” are used interchangeably herein to refer to an organism too small to be seen clearly with the naked eye, including, but not limited to, microscopic bacteria, fungi (molds), algae, protozoa, and viruses.

The term “modulate” as used herein means to regulate, alter, adapt, or adjust to a certain measure or proportion.

The terms “parenchyma” or “parenchymal” as used herein refer to the distinguishing cells or tissue constituting an organ, e.g. cerebral or brain parenchyma refers to the distinguishing cells constituting cerebral or brain tissue.

The terms “parenteral” or “parenterally” as used herein refer to introduction into the body by way of an injection (i.e., administration by injection), including, but not limited to, infusion techniques. Non-limiting examples of parenteral administration includes, but are not limited to, intravascular infusion or direct injection into brain or spinal cord parenchyma. When used generally, the term “parenteral” refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents. Additional administration may be performed, for example, intravenously, pericardially, orally, via implant, transmucosally, transdermally, intramuscularly, subcutaneously, intraperitoneally, intrathecally, intralymphatically, intralesionally, or epidurally. Administering can be performed, for example, once, a plurality of times, and/or over one or more extended periods either as individual unit doses or in the form of a treatment regimen comprising multiple unit doses of multiple drugs and/or substances.

As used herein, the term “potent” or “potency” refers to the necessary biological activity of the chemotactic hematopoietic stem cell product of the present invention, i.e., potent cells of the present invention remain viable, are capable of mediated mobility, and are able to grow, i.e., to form hematopoietic colonies in an in vitro CFU assay.

The term “traumatic penumbra” or “traumatic penumbra region” as used herein refer to a tissue that is most at risk of secondary ischemic injury and that will be most affected by changes in physiology or therapeutic interventions.

The term “progenitor cell” as used herein refers to an immature cell in the bone marrow that can be isolated by growing suspensions of marrow cells in culture dishes with added growth factors. Progenitor cells mature into precursor cells that mature into blood cells. Progenitor cells are referred to as colony-forming units (CFU) or colony-forming cells (CFC). The specific lineage of a progenitor cell is indicated by a suffix, such as, but not limited to, CFU-E (erythrocytic), CFU-GM (granulocytic/macrophage), and CFU-GEMM (pluripotent hematopoietic progenitor).

The term “repair” as used herein as a noun refers to any correction, reinforcement, reconditioning, remedy, making up for, making sound, renewal, mending, patching, or the like that restores function. When used as a verb, it means to correct, to reinforce, to recondition, to remedy, to make up for, to make sound, to renew, to mend, to patch or to otherwise restore function. According to some embodiments “repair” includes full repair and partial repair.

The term “Sca-1” or “stem cell antigen-1” refers to a surface protein component in a signaling pathway that affects the self-renewal ability of mesenchymal stem cells.

The term “stem cells” refers to undifferentiated cells having high proliferative potential with the ability to self-renew that can generate daughter cells that can undergo terminal differentiation into more than one distinct cell phenotype.

The term “stent” is used to refer to a small tube used to prop open an artery. The stent is collapsed to a small diameter, put over a balloon catheter, inserted through a main artery in the groin (femoral artery) or arm (brachial artery) and threaded up to the narrowed/blocked section of the artery. When it reaches the right location, the balloon is inflated slightly to push any plaque out of the way and to expand the artery (balloon angioplasty). When the balloon is inflated, the stent expands, locks in place, and forms a scaffold to hold the artery open. The stent stays in the artery permanently. In certain subjects, a stent reduces the renarrowing that occurs after balloon angioplasty or other procedures that use catheters. A stent also may help restore normal blood flow and keep an artery open if it has been torn or injured by the balloon catheter. Reclosure (restenosis) may be a problem with the stent procedure. Drug-eluting stents are stents coated with drugs that are slowly released. These drugs may help keep the blood vessel from reclosing.

The term “sterotactically-guided injection” as used herein refers to an injection guided by a stereotactic apparatus. The term “stereotactic apparatus” as used herein refers to equipment used for precise control of a probe to be inserted into a patient's brain, brainstem, or spinal cord. Examples of stereotactic apparatus include stereotactic frames that attach to a patient's head, adapters, such as may be used to mount devices to a stereotactic frame, microdrives for precision movement of a probe generally along the longitudinal axis of the probe, translation stages for lateral positioning of a probe, and other devices attached to or used in conjunction with a stereotactic frame.

The term “subject” or “patient” as used herein includes animal species of mammalian origin, including humans.

The term “Thy-1” refers to the Ig superfamily cell surface glycoprotein Thy-1 expressed on immune cells and neurons of rodents and humans, which is hypothesized to function in cell adhesion and signal transduction in T cell differentiation, proliferation, and apoptosis.

As used herein, the term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

The term “vascular insufficiency” refers to insufficient blood flow.

### I. Composition for Treating Traumatic Brain or Spinal Cord Injury

According to one aspect, the present invention provides a pharmaceutical composition for treating a vascular insufficiency following a traumatic brain or spinal cord injury.

According to some embodiments, the traumatic brain injury comprises a closed head injury, a concussive head injury, or a penetrating head injury. According to some embodiments, the traumatic brain injury is a severe traumatic brain injury (for example, an acute subdural hematoma or a parenchymal hematoma) that causes an increase in intracranial pressure.

According to some other embodiments, the traumatic brain injury is a mild or mild-to-moderate closed head injuries (such as a cerebral concussion, cerebral contusion, epidural hematoma, subdural hematoma, intraventricular hemorrhage, and diffuse axonal injury) that do not require craniectomy.

According to one embodiment, the pharmaceutical composition for treating traumatic brain or spinal cord injury of the present invention comprises a chemotactic hematopoietic stem cell product comprising a nonexpanded, isolated population of autologous mononuclear cells. According to one embodiment, the mononuclear cells are enriched for CD34+ cells. According to one embodiment, the CD34+ cells further contain a subpopulation of potent SDF-1 mobile CD34+/CXCR-4+ cells that have CXCR-4-mediated chemotactic activity.

According to another embodiment, the chemotactic hematopoietic stem cell product is prepared by isolating or purifying CD34+ hematopoietic stem cells from a population of mononuclear cells isolated from bone marrow harvested from the subject. According to another embodiment, the chemotactic hematopoietic stem cell product is prepared by isolating or purifying CD34+ hematopoietic stem cells from a population of mononuclear cells isolated from peripheral blood collected from the subject.

According to another embodiment, the chemotactic hematopoietic stem cell product is prepared by isolating or purifying CD34+ hematopoietic stem cells from a population of mononuclear cells isolated from peripheral blood collected from the subject, after treatment with a hematopoietic stem cell mobilizing agent. According to some such embodiments, the hematopoietic stem cell mobilizing agent comprises G-CSF, GM-CSF, or a pharmaceutically acceptable analog or derivative thereof. According to some embodiments, the hematopoietic stem cell mobilizing agent is a recombinant analog or derivative of a colony stimulating factor. According to some embodiments, the hematopoietic stem cell mobilizing agent is filgrastim. According to some embodiments, the hematopoietic stem cell mobilizing agent is mobilzil.

According to another embodiment, the isolated population of autologous mononuclear cells is enriched for CD34+ cells. According to one embodiment, the CD34+ cells further contain a subpopulation of potent SDF-1 mobile CD34+/CXCR-4+ cells. According to another embodiment, the subpopulation of potent SDF-1 mobile CD34+/CXCR-4+ cells has CXCR-4-mediated chemotactic activity.

According to another embodiment, the nonexpanded isolated population of autologous mononuclear cells is purified from cellular components of a bone marrow aspirate acquired from the subject. According to another embodiment, the nonexpanded isolated population of autologous mononuclear cells is purified from peripheral blood. According to one embodiment, the nonexpanded, isolated population of autologous mononuclear cells is enriched for CD34+ cells. According to another embodiment, the CD34+ cells further contain a subpopulation of potent SDF-1 mobile CD34+/CXCR-4+ cells. According to another embodiment, the subpopulation of potent SDF-1 mobile CD34+/CXCR-4+ cells has CXCR-4-mediated chemotactic activity.

According to some embodiments, the chemotactic hematopoietic stem cell product enriched for CD34+ cells contains at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% pure CD34+ cells.

According to some embodiments, the subpopulation of CD34+ cells comprises from at least about 1% to at least about 95% of the isolated population of autologous mononuclear cells. According to one embodiment, the subpopulation of CD34+ cells comprises at least about 1% of the isolated population of autologous mononuclear cells. According to one embodiment, the subpopulation of CD34+ cells comprises at least about 2% of the isolated population of autologous mononuclear cells. According to one embodiment, the subpopulation of CD34+ cells comprises at least about 3% of the isolated population of autologous mononuclear cells. According to one embodiment, the subpopulation of CD34+ cells comprises at least about 4% of the isolated population of autologous mononuclear cells. According to one embodiment, the subpopulation of CD34+ cells comprises at least about 5% of the isolated population of autologous mononuclear cells. According to one embodiment, the subpopulation of CD34+ cells comprises at least about 6% of the isolated population of autologous mononuclear cells. According to one embodiment, the subpopulation of CD34+ cells comprises at least about 7% of the isolated population of autologous mononuclear cells. According to one embodiment, the subpopulation of CD34+ cells comprises at least about 8% of the isolated population of autologous mononuclear cells. According to one embodiment, the subpopulation of CD34+ cells comprises at least about 9% of the isolated population of autologous mononuclear cells. According to one embodiment, the subpopulation of CD34+ cells comprises at least about 10% of the isolated population of autologous mononuclear cells. According to one embodiment, the subpopulation of CD34+ cells comprises at least about 15% of the isolated population of autologous mononuclear cells. According to one embodiment, the subpopulation of CD34+ cells comprises at least about 20% of the isolated population of autologous mononuclear cells. According to one embodiment, the population of CD34+ cells comprises at least about 25% of the isolated population of autologous mononuclear cells. According to one embodiment, the subpopulation of CD34+ cells comprises at least about 30% of the isolated population of autologous mononuclear cells. According to one embodiment, the subpopulation of CD34+ cells comprises at least about 35% of the isolated population of autologous mononuclear cells. According to one embodiment, the subpopulation of CD34+ cells comprises at least about 40% of the isolated population of autologous mononuclear cells. According to one embodiment, the subpopulation of CD34+ cells comprises at least about 45% of the isolated population of autologous mononuclear cells. According to one embodiment, the subpopulation of CD34+ cells comprises at least about 50% of the isolated population of autologous mononuclear cells. According to one embodiment, the subpopulation of CD34+ cells comprises at least about 55% of the isolated population of autologous mononuclear cells. According to one embodiment, the subpopulation of CD34+ cells comprises at least about 60% of the isolated population of autologous mononuclear cells. According to one embodiment, the subpopulation of CD34+ cells comprises at least about 65% of the isolated population of autologous mononuclear cells. According to one embodiment, the subpopulation of CD34+ cells comprises at least about 70% of the isolated population of autologous mononuclear cells. According to one embodiment, the subpopulation of CD34+ cells comprises at least about 75% of the isolated population of autologous mononuclear cells. According to one embodiment, the subpopulation of CD34+ cells comprises at least about 80% of the isolated population of autologous mononuclear cells.

According to one embodiment, the subpopulation of CD34+ cells comprises at least about 85% of the isolated population of autologous mononuclear cells. According to one embodiment, the subpopulation of CD34+ cells comprises at least about 90% of the isolated population of autologous mononuclear cells. According to one embodiment, the subpopulation of CD34+ cells comprises at least about 95% of the isolated population of autologous mononuclear cells.

According to some embodiments, the chemotactic activity of the CD34+/CXCR-4+ cells is mediated by SDF-1, VEGF, and/or CXCR-4. According to one embodiment, the chemotactic activity of the CD34+/CXCR-4+ cells is mediated by SDF-1. According to one embodiment, the chemotactic activity of the CD34+/CXCR-4+ cells is mediated by VEGF. According to one embodiment, the chemotactic activity of the CD34+/CXCR-4+ cells is mediated by CXCR-4.

According to another embodiment, at least about 1% to at least about 95% of the CD34+ cells are viable for at least about 24, at least about 48 hours, or at least about 72 hours following acquisition of the nonexpanded isolated population of autologous mononuclear cells.

According to some embodiments, at least about 1% to at least about 95% of the CD34+ cells are viable for at least about 24 hours following acquisition of the isolated population of autologous mononuclear cells comprising the subpopulation of CD34+ cells. According to one embodiment, at least about 1% the CD34+ cells are viable for at least about 24 hours following acquisition of the isolated population of autologous mononuclear cells comprising the subpopulation of CD34+ cells. According to one embodiment, at least about 2% the CD34+ cells are viable for at least about 24 hours following acquisition of the isolated population of autologous mononuclear cells comprising the subpopulation of CD34+ cells. According to one embodiment, at least about 3% the CD34+ cells are viable for at least about 24 hours following acquisition of the isolated population of autologous mononuclear cells comprising the subpopulation of CD34+ cells. According to one embodiment, at least about 4% the CD34+ cells are viable for at least about 24 hours following acquisition of the isolated population of autologous mononuclear cells comprising the subpopulation of CD34+ cells. According to one embodiment, at least about 5% the CD34+ cells are viable for at least about 24 hours following acquisition of the isolated population of autologous mononuclear cells comprising the subpopulation of CD34+ cells. According to one embodiment, at least about 6% the CD34+ cells are viable for at least about 24 hours following acquisition of the isolated population of autologous mononuclear cells comprising the subpopulation of CD34+ cells. According to one embodiment, at least about 7% the CD34+ cells are viable for at least about 24 hours following acquisition of the isolated population of autologous mononuclear cells comprising the subpopulation of CD34+ cells. According to one embodiment, at least about 8% the CD34+ cells are viable for at least about 24 hours following acquisition of the isolated population of autologous mononuclear cells comprising the subpopulation of CD34+ cells. According to one embodiment, at least about 9% the CD34+ cells are viable for at least about 24 hours following acquisition of the isolated population of autologous mononuclear cells comprising the subpopulation of CD34+ cells. According to one embodiment, at least about 10% the CD34+ cells are viable for at least about 24 hours following acquisition of the isolated population of autologous mononuclear cells comprising the subpopulation of CD34+ cells. According to one embodiment, at least about 15% the CD34+ cells are viable for at least about 24 hours following acquisition of the isolated population of autologous mononuclear cells comprising the subpopulation of CD34+ cells. According to one embodiment, at least about 20% the CD34+ cells are viable for at least about 24 hours following acquisition of the isolated population of autologous mononuclear cells comprising the subpopulation of CD34+ cells. ccording to one embodiment, at least about 25% the CD34+ cells are viable for at least about 24 hours following acquisition of the isolated population of autologous mononuclear cells comprising the subpopulation of CD34+ cells. According to one embodiment, at least about 30% the CD34+ cells are viable for at least about 24 hours following acquisition of the isolated population of autologous mononuclear cells comprising the subpopulation of CD34+ cells. According to one embodiment, at least about 35% the CD34+ cells are viable for at least about 24 hours following acquisition of the isolated population of autologous mononuclear cells comprising the subpopulation of CD34+ cells. According to one embodiment, at least about 40% the CD34+ cells are viable for at least about 24 hours following acquisition of the isolated population of autologous mononuclear cells comprising the subpopulation of CD34+ cells. According to one embodiment, at least about 45% the CD34+ cells are viable for at least about 24 hours following acquisition of the isolated population of autologous mononuclear cells comprising the subpopulation of CD34+ cells. According to one embodiment, at least about 50% the CD34+ cells are viable for at least about 24 hours following acquisition of the isolated population of autologous mononuclear cells comprising the subpopulation of CD34+ cells. According to one embodiment, at least about 55% the CD34+ cells are viable for at least about 24 hours following acquisition of the isolated population of autologous mononuclear cells comprising the subpopulation of CD34+ cells. According to one embodiment, at least about 60% the CD34+ cells are viable for at least about 24 hours following acquisition of the isolated population of autologous mononuclear cells comprising the subpopulation of CD34+ cells. According to one embodiment, at least about 65% the CD34+ cells are viable for at least about 24 hours following acquisition of the isolated population of autologous mononuclear cells comprising the subpopulation of CD34+ cells. According to one embodiment, at least about 70% the CD34+ cells are viable for at least about 24 hours following acquisition of the isolated population of autologous mononuclear cells comprising the subpopulation of CD34+ cells. According to one embodiment, at least about 75% the CD34+ cells are viable for at least about 24 hours following acquisition of the isolated population of autologous mononuclear cells comprising the subpopulation of CD34+ cells. According to one embodiment, at least about 80% the CD34+ cells are viable for at least about 24 hours following acquisition of the isolated population of autologous mononuclear cells comprising the subpopulation of CD34+ cells. According to one embodiment, at least about 85% the CD34+ cells are viable for at least about 24 hours following acquisition of the isolated population of autologous mononuclear cells comprising the subpopulation of CD34+ cells. According to one embodiment, at least about 90% the CD34+ cells are viable for at least about 24 hours following acquisition of the isolated population of autologous mononuclear cells comprising the subpopulation of CD34+ cells. According to one embodiment, at least about 95% the CD34+ cells are viable for at least about 24 hours following acquisition of the isolated population of autologous mononuclear cells comprising the subpopulation of CD34+ cells.

According to some embodiments, at least about 1% to at least about 95% of the CD34+ cells are viable for at least about 48 hours following acquisition of the isolated population of autologous mononuclear cells comprising the subpopulation of CD34+ cells. According to one embodiment, at least about 1% the CD34+ cells are viable for at least about 48 hours following acquisition of the isolated population of autologous mononuclear cells comprising the subpopulation of CD34+ cells. According to one embodiment, at least about 2% the CD34+ cells are viable for at least about 48 hours following acquisition of the isolated population of autologous mononuclear cells comprising the subpopulation of CD34+ cells. According to one embodiment, at least about 3% the CD34+ cells are viable for at least about 48 hours following acquisition of the isolated population of autologous mononuclear cells comprising the subpopulation of CD34+ cells. According to one embodiment, at least about 4% the CD34+ cells are viable for at least about 48 hours following acquisition of the isolated population of autologous mononuclear cells comprising the subpopulation of CD34+ cells. According to one embodiment, at least about 5% the CD34+ cells are viable for at least about 48 hours following acquisition of the isolated population of autologous mononuclear cells comprising the subpopulation of CD34+ cells. According to one embodiment, at least about 6% the CD34+ cells are viable for at least about 48 hours following acquisition of the isolated population of autologous mononuclear cells comprising the subpopulation of CD34+ cells. According to one embodiment, at least about 7% the CD34+ cells are viable for at least about 48 hours following acquisition of the isolated population of autologous mononuclear cells comprising the subpopulation of CD34+ cells. According to one embodiment, at least about 8% the CD34+ cells are viable for at least about 48 hours following acquisition of the isolated population of autologous mononuclear cells comprising the subpopulation of CD34+ cells. According to one embodiment, at least about 9% the CD34+ cells are viable for at least about 48 hours following acquisition of the isolated population of autologous mononuclear cells comprising the subpopulation of CD34+ cells. According to one embodiment, at least about 10% the CD34+ cells are viable for at least about 48 hours following acquisition of the isolated population of autologous mononuclear cells comprising the subpopulation of CD34+ cells. According to one embodiment, at least about 15% the CD34+ cells are viable for at least about 48 hours following acquisition of the isolated population of autologous mononuclear cells comprising the subpopulation of CD34+ cells. According to one embodiment, at least about 20% the CD34+ cells are viable for at least about 48 hours following acquisition of the isolated population of autologous mononuclear cells comprising the subpopulation of CD34+ cells. According to one embodiment, at least about 25% the CD34+ cells are viable for at least about 48 hours following acquisition of the isolated population of autologous mononuclear cells comprising the subpopulation of CD34+ cells. According to one embodiment, at least about 30% the CD34+ cells are viable for at least about 48 hours following acquisition of the isolated population of autologous mononuclear cells comprising the subpopulation of CD34+ cells. According to one embodiment, at least about 35% the CD34+ cells are viable for at least about 48 hours following acquisition of the isolated population of autologous mononuclear cells comprising the subpopulation of CD34+ cells. According to one embodiment, at least about 40% the CD34+ cells are viable for at least about 48 hours following acquisition of the isolated population of autologous mononuclear cells comprising the subpopulation of CD34+ cells. According to one embodiment, at least about 45% the CD34+ cells are viable for at least about 48 hours following acquisition of the isolated population of autologous mononuclear cells comprising the subpopulation of CD34+ cells. According to one embodiment, at least about 50% the CD34+ cells are viable for at least about 48 hours following acquisition of the isolated population of autologous mononuclear cells comprising the subpopulation of CD34+ cells. According to one embodiment, at least about 55% the CD34+ cells are viable for at least about 48 hours following acquisition of the isolated population of autologous mononuclear cells comprising the subpopulation of CD34+ cells. According to one embodiment, at least about 60% the CD34+ cells are viable for at least about 48 hours following acquisition of the isolated population of autologous mononuclear cells comprising the subpopulation of CD34+ cells. According to one embodiment, at least about 65% the CD34+ cells are viable for at least about 48 hours following acquisition of the isolated population of autologous mononuclear cells comprising the subpopulation of CD34+ cells. According to one embodiment, at least about 70% the CD34+ cells are viable for at least about 48 hours following acquisition of the isolated population of autologous mononuclear cells comprising the subpopulation of CD34+ cells. According to one embodiment, at least about 75% the CD34+ cells are viable for at least about 48 hours following acquisition of the isolated population of autologous mononuclear cells comprising the subpopulation of CD34+ cells. According to one embodiment, at least about 80% the CD34+ cells are viable for at least about 48 hours following acquisition of the isolated population of autologous mononuclear cells comprising the subpopulation of CD34+ cells. According to one embodiment, at least about 85% the CD34+ cells are viable for at least about 48 hours following acquisition of the isolated population of autologous mononuclear cells comprising the subpopulation of CD34+ cells. According to one embodiment, at least about 90% the CD34+ cells are viable for at least about 48 hours following acquisition of the isolated population of autologous mononuclear cells comprising the subpopulation of CD34+ cells. According to one embodiment, at least about 95% the CD34+ cells are viable for at least about 48 hours following acquisition of the isolated population of autologous mononuclear cells comprising the subpopulation of CD34+ cells.

According to some embodiments, at least about 1% to at least about 95% of the CD34+ cells are viable for at least about 72 hours following acquisition of the isolated population of autologous mononuclear cells comprising the subpopulation of CD34+ cells. According to one embodiment, at least about 1% the CD34+ cells are viable for at least about 72 hours following acquisition of the isolated population of autologous mononuclear cells comprising the subpopulation of CD34+ cells. According to one embodiment, at least about 2% the CD34+ cells are viable for at least about 72 hours following acquisition of the isolated population of autologous mononuclear cells comprising the subpopulation of CD34+ cells. According to one embodiment, at least about 3% the CD34+ cells are viable for at least about 72 hours following acquisition of the isolated population of autologous mononuclear cells comprising the subpopulation of CD34+ cells. According to one embodiment, at least about 4% the CD34+ cells are viable for at least about 72 hours following acquisition of the isolated population of autologous mononuclear cells comprising the subpopulation of CD34+ cells. According to one embodiment, at least about 5% the CD34+ cells are viable for at least about 72 hours following acquisition of the isolated population of autologous mononuclear cells comprising the subpopulation of CD34+ cells. According to one embodiment, at least about 6% the CD34+ cells are viable for at least about 72 hours following acquisition of the isolated population of autologous mononuclear cells comprising the subpopulation of CD34+ cells. According to one embodiment, at least about 7% the CD34+ cells are viable for at least about 72 hours following acquisition of the isolated population of autologous mononuclear cells comprising the subpopulation of CD34+ cells. According to one embodiment, at least about 8% the CD34+ cells are viable for at least about 72 hours following acquisition of the isolated population of autologous mononuclear cells comprising the subpopulation of CD34+ cells. According to one embodiment, at least about 9% the CD34+ cells are viable for at least about 72 hours following acquisition of the isolated population of autologous mononuclear cells comprising the subpopulation of CD34+ cells. According to one embodiment, at least about 10% the CD34+ cells are viable for at least about 72 hours following acquisition of the isolated population of autologous mononuclear cells comprising the subpopulation of CD34+ cells. According to one embodiment, at least about 15% the CD34+ cells are viable for at least about 72 hours following acquisition of the isolated population of autologous mononuclear cells comprising the subpopulation of CD34+ cells. According to one embodiment, at least about 20% the CD34+ cells are viable for at least about 72 hours following acquisition of the isolated population of autologous mononuclear cells comprising the subpopulation of CD34+ cells. According to one embodiment, at least about 25% the CD34+ cells are viable for at least about 72 hours following acquisition of the isolated population of autologous mononuclear cells comprising the subpopulation of CD34+ cells. According to one embodiment, at least about 30% the CD34+ cells are viable for at least about 72 hours following acquisition of the isolated population of autologous mononuclear cells comprising the subpopulation of CD34+ cells. According to one embodiment, at least about 35% the CD34+ cells are viable for at least about 72 hours following acquisition of the isolated population of autologous mononuclear cells comprising the subpopulation of CD34+ cells. According to one embodiment, at least about 40% the CD34+ cells are viable for at least about 72 hours following acquisition of the isolated population of autologous mononuclear cells comprising the subpopulation of CD34+ cells. According to one embodiment, at least about 45% the CD34+ cells are viable for at least about 72 hours following acquisition of the isolated population of autologous mononuclear cells comprising the subpopulation of CD34+ cells. According to one embodiment, at least about 50% the CD34+ cells are viable for at least about 72 hours following acquisition of the isolated population of autologous mononuclear cells comprising the subpopulation of CD34+ cells. According to one embodiment, at least about 55% the CD34+ cells are viable for at least about 72 hours following acquisition of the isolated population of autologous mononuclear cells comprising the subpopulation of CD34+ cells. According to one embodiment, at least about 60% the CD34+ cells are viable for at least about 72 hours following acquisition of the isolated population of autologous mononuclear cells comprising the subpopulation of CD34+ cells. According to one embodiment, at least about 65% the CD34+ cells are viable for at least about 72 hours following acquisition of the isolated population of autologous mononuclear cells comprising the subpopulation of CD34+ cells. According to one embodiment, at least about 70% the CD34+ cells are viable for at least about 72 hours following acquisition of the isolated population of autologous mononuclear cells comprising the subpopulation of CD34+ cells. According to one embodiment, at least about 75% the CD34+ cells are viable for at least about 72 hours following acquisition of the isolated population of autologous mononuclear cells comprising the subpopulation of CD34+ cells. According to one embodiment, at least about 80% the CD34+ cells are viable for at least about 72 hours following acquisition of the isolated population of autologous mononuclear cells comprising the subpopulation of CD34+ cells. According to one embodiment, at least about 85% the CD34+ cells are viable for at least about 72 hours following acquisition of the isolated population of autologous mononuclear cells comprising the subpopulation of CD34+ cells. According to one embodiment, at least about 90% the CD34+ cells are viable for at least about 72 hours following acquisition of the isolated population of autologous mononuclear cells comprising the subpopulation of CD34+ cells. According to one embodiment, at least about 95% the CD34+ cells are viable for at least about 72 hours following acquisition of the isolated population of autologous mononuclear cells comprising the subpopulation of CD34+ cells.

According to another embodiment, the CD34+ cells can form hematopoietic colonies in vitro for at least about 4 hours, at least about 8 hours, at least about 16 hours, at least about 20 hours, at least about 24 hours, at least about 36 hours, at least about 48 hours, at least about 60 hours, or at least about 72 hours following acquisition of the enriched population of CD34+ cells. According to another embodiment, the CD34+ cells can form hematopoietic colonies in vitro for at least about 4 hours, at least about 8 hours, at least about 16 hours, at least about 20 hours, at least about 24 hours, at least about 36 hours, at least about 48 hours, at least about 60 hours, or at least about 72 hours following acquisition of the isolated population of autologous mononuclear cells comprising the subpopulation of CD34+ cells. According to one embodiment, the CD34+ cells can form hematopoietic colonies in vitro for at least about 4 hours following acquisition of the isolated population of autologous mononuclear cells comprising the subpopulation of CD34+ cells. According to one embodiment, the CD34+ cells can form hematopoietic colonies in vitro for at least about 8 hours following acquisition of the isolated population of autologous mononuclear cells comprising the subpopulation of CD34+ cells. According to one embodiment, the CD34+ cells can form hematopoietic colonies in vitro for at least about 16 hours following acquisition of the isolated population of autologous mononuclear cells comprising the subpopulation of CD34+ cells. According to one embodiment, the CD34+ cells can form hematopoietic colonies in vitro for at least about 20 hours following acquisition of the isolated population of autologous mononuclear cells comprising the subpopulation of CD34+ cells. According to one embodiment, the CD34+ cells can form hematopoietic colonies in vitro for at least about 24 hours following acquisition of the isolated population of autologous mononuclear cells comprising the subpopulation of CD34+ cells. According to one embodiment, the CD34+ cells can form hematopoietic colonies in vitro for at least about 36 hours following acquisition of the isolated population of autologous mononuclear cells comprising the subpopulation of CD34+ cells. According to one embodiment, the CD34+ cells can form hematopoietic colonies in vitro for at least about 48 hours following acquisition of the isolated population of autologous mononuclear cells comprising the subpopulation of CD34+ cells. According to one embodiment, the CD34+ cells can form hematopoietic colonies in vitro for at least about 60 hours following acquisition of the isolated population of autologous mononuclear cells comprising the subpopulation of CD34+ cells. According to one embodiment, the CD34+ cells can form hematopoietic colonies in vitro for at least about 72 hours following acquisition of the isolated population of autologous mononuclear cells comprising the subpopulation of CD34+ cells.

CD34+ cells can be enriched/selected by any techniques known to the skilled artisan. For example, according to some embodiments, the population of mononuclear cells comprising CD34+ cells is enriched for cells expressing CD34 cell antigen and CXCR-4 cell antigen by fluorescence activated cell sorting (FACS). According to some embodiments, CD34+ cells in the bone marrow or peripheral blood are enriched/selected by positive or negative immunoseparation techniques. According to some embodiments, isolation and/or purification of hematopoietic stem cells from the bone marrow or peripheral blood is based on cell fractionation methods based on size and cell density, efflux of metabolic dyes, or resistance to cytotoxic agents. According to one embodiment, for example, CD34+ cells in the bone marrow or peripheral blood are enriched/selected using a monoclonal anti-CD34 antibody and an immunomagnetic separation technique.

The selected CD34+ cells can be identified, quantified and characterized by techniques known in the art. For example, according to some embodiments, the percentage of CD34+ cells in the bone marrow, blood, or the chemotactic hematopoietic stem cell product can be determined by FACS analysis. According to another embodiment, CD34 protein expression is quantified by Western blot. The term “Western blot” refers to a method for identifying proteins in a complex mixture; proteins are separated electrophoretically in a gel medium; transferred from the gel to a protein binding sheet or membrane; and the sheet or membrane containing the separated proteins exposed to specific antibodies which bind to, locate, and enable visualization of protein(s) of interest. For example, monoclonal anti-CD34 antibody can be used to detect CD34 protein adhered to a membrane in situ.

According to another embodiment, the expression of CD34 mRNA and DNA in the isolated CD34+ cells can be quantified. The term “northern blot” as used herein refers to a technique in which RNA from a specimen is separated into its component parts on a gel by electrophoresis and transferred to a specifically modified paper support so that the mRNA is fixed in its electrophoretic positions. CD34 related sequences are identified using probes comprising a reporter molecule, such as, without limitation, a radioactive label. According to another embodiment, the level of CD34 and/or CXCR-4 expression is/are determined by quantitative or semi-quantitative PCR or real-time PCR (“qPCR”) techniques. The abbreviation “PCR” refers to polymerase chain reaction, which is a technique for amplifying the quantity of DNA, thus making the DNA easier to isolate, clone and sequence. See, e.g., U.S. Pat. Nos. 5,656,493, 5,333,675, 5,234,824, and 5,187,083, each of which is incorporated herein by reference.

According to another embodiment, the selected CD34+ hematopoietic stem cells of the chemotactic hematopoietic stem cell product of the present invention contain a subpopulation of CD34+/CXCR-4+ cells having CXCR-4 mediated chemotactic activity. According to one embodiment, the hematopoietic stem cell product of the present invention comprises a minimum number of isolated CD34+ hematopoietic stem cells such that a subpopulation of at least 0.5×10⁶ potent SDF-1 mobile CD34+/CXCR-4+ cells having CXCR-4-mediated chemotactic activity is present.

According to another embodiment, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, or 34% of the CXCR-4-mediated chemotactic activity of the subpopulation of potent SDF-1 mobile CD34+/CXCR-4+ cells is retained for at least 4 hours, at least 8 hours, at least 16 hours, at least 20 hours, at least 24 hours, at least 36 hours, at least 48 hours, at least 60 hours, or at least 72 hours following acquisition of the isolated population of autologous mononuclear cells comprising a subpopulation of CD34+ cells.

According to another embodiment, at least an average of about 5%, 6%, 7%, 8%, 9%, 10%, 11%, 12%, 13%, 14%, 14%, 16% or 17% of the CXCR-4 mediated chemotactic activity of the subpopulation of potent SDF-1 mobile CD34+/CXCR-4+ cells is retained for at least 4 hours, at least 8 hours, at least 16 hours, at least 20 hours, at least 24 hours, at least 36 hours, at least 48 hours, at least 60 hours, or at least 72 hours following acquisition of the isolated population of autologous mononuclear cells comprising a subpopulation of CD34+ cells. According to another embodiment, the CD34+/CXCR-4+ cells of the chemotactic hematopoietic cell product retain at least about 2% of their CXCR-4 mediated chemotactic activity for at least 4 hours, at least 8 hours, at least 16 hours, at least 20 hours, at least 24 hours, at least 36 hours, at least 48 hours, at least 60 hours, or 72 hours following acquisition of the isolated population of autologous mononuclear cells comprising a subpopulation of CD34+ cells further containing a subpopulation of potent SDF-1 mobile CD34+/CXCR-4+ cells.

According to one embodiment, at least about 1% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 2% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ having CXCR-4-mediated chemotactic activity. According to another embodiment, at least about 3% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 4% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 5% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 6% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 7% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 8% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 9% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 10% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 11% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 12% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 13% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 14% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 15% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 16% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 17% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 18% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 19% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 20% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 21% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 22% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 23% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 24% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 25% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 26% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 27% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 28% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 29% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 30% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 31% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 32% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 33% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 34% of the CXCR-4 mediated chemotactic activity of the potent SDF-1 mobile CD34+/CXCR-4+ cells is retained for at least 24 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity.

According to another embodiment, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, or 34% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 2% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 3% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 4% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 5% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 6% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 7% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 8% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 9% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 10% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 11% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 12% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 13% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 14% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 15% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 16% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 17% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 18% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 19% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 20% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 21% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 22% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 23% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 24% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 25% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 26% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 27% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 28% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 29% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 30% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 31% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 32% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 33% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 34% of the CXCR-4 mediated chemotactic activity of the potent SDF-1 mobile CD34+/CXCR-4+ cells is retained for at least 48 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity.

According to one embodiment, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, or 34% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 2% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 3% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 4% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 5% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 6% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 7% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 8% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 9% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 10% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 11% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 12% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 13% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 14% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 15% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 16% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 17% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 18% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 19% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 20% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 21% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 22% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 23% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 24% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 25% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 26% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 27% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 28% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 29% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 30% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 31% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 32% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 33% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity. According to another embodiment, at least about 34% of the CXCR-4 mediated chemotactic activity of the potent SDF-1 mobile CD34+/CXCR-4+ cells is retained for at least 72 hours following acquisition of the isolated population of autologous mononuclear cells comprising the subpopulation of CD34+ cells further containing a subpopulation of potent SDF-1 mobile CD34+/CXCR-4+ cells having CXCR-4-mediated chemotactic activity.

According to some embodiments, the pharmaceutical composition of the present invention further comprises a stabilizing amount of serum. According to some such embodiments, the stabilizing amount of serum is from at least about 0.1% (v/v) to about 70% (v/v). According to another embodiment, the stabilizing amount of serum is at least about 0.1% (v/v). According to another embodiment, the stabilizing amount of serum is at least about 0.5% (v/v). According to another embodiment, the stabilizing amount of serum is at least about 1% (v/v). According to another embodiment, the stabilizing amount of serum is at least about 2% (v/v). According to another embodiment, the stabilizing amount of serum is at least about 3% (v/v). According to another embodiment, the stabilizing amount of serum is at least about 4% (v/v). According to another embodiment, the stabilizing amount of serum is at least about 5% (v/v). According to another embodiment, the stabilizing amount of serum is at least about 6% (v/v). According to another embodiment, the stabilizing amount of serum is at least about 7% (v/v). According to another embodiment, the stabilizing amount of serum is at least about 8% (v/v). According to another embodiment, the stabilizing amount of serum is at least about 9% (v/v). According to another embodiment, the stabilizing amount of serum is at least about 10% (v/v). According to another embodiment, the stabilizing amount of serum is at least about 15% (v/v). According to another embodiment, the stabilizing amount of serum is at least about 20% (v/v). According to another embodiment, the stabilizing amount of serum is at least about 25% (v/v). According to another embodiment, the stabilizing amount of serum is at least about 30% (v/v). According to another embodiment, the stabilizing amount of serum is at least about 35% (v/v). According to another embodiment, the stabilizing amount of serum is at least about 40% (v/v). According to another embodiment, the stabilizing amount of serum is at least about 45% (v/v). According to another embodiment, the stabilizing amount of serum is at least about 50% (v/v). According to another embodiment, the stabilizing amount of serum is at least about 55% (v/v). According to another embodiment, the stabilizing amount of serum is at least about 60% (v/v). According to another embodiment, the stabilizing amount of serum is at least about 65% (v/v). According to another embodiment, the stabilizing amount of serum is at least about 70% (v/v).

According to some embodiments, the pharmaceutical composition of the present invention further comprises serum at a concentration of at least 10% by volume of the composition. According to one embodiment, the serum is autologous. According to another embodiment, the serum is a synthetic or recombinant serum. The minimum concentration of serum present in the composition is at least about 10%, 15%, 20%, 25%, 30%, 35%, 40%, 45%, 50%, 55%, 60%, 65%, or 70% expressed as ml/100 cc final volume of the composition. The maximum concentration of serum present in the composition of the present invention is about 70%, 65%, 60%, 55%, 50%, 45%, 40%, 35%, 30%, 25%, 20%, 15%, or 10% expressed as ml/100 cc final volume of the composition.

According to some embodiments, the composition of the present invention may be formulated with an excipient, carrier or vehicle including, but not limited to, a solvent. The terms “excipient”, “carrier”, or “vehicle” as used herein refers to carrier materials suitable for formulation and administration of the chemotactic hematopoietic stem cell product described herein. Carriers and vehicles useful herein include any such materials known in the art which are nontoxic and do not interact with other components. As used herein the phrase “pharmaceutically acceptable carrier” refers to any substantially non-toxic carrier useable for formulation and administration of the composition of the present invention in which the chemotactic hematopoietic stem cell product of the present invention will remain stable and bioavailable.

The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. For example, the pharmaceutically acceptable carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable pharmaceutically acceptable carriers for the compositions of the present invention include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like. Such carrier solutions also can contain buffers, diluents and other suitable additives. The term “buffer” as used herein refers to a solution or liquid whose chemical makeup neutralizes acids or bases without a significant change in pH. Examples of buffers envisioned by the present invention include, but are not limited to, Dulbecco's phosphate buffered saline (PBS), Ringer's solution, 5% dextrose in water (D5W), normal/physiologic saline (0.9% NaCl). According to some embodiments, the infusion solution is isotonic to subject tissues. According to some embodiments, the infusion solution is hypertonic to subject tissues. Compositions of the present invention that are for parenteral administration, can include pharmaceutically acceptable carriers such as sterile aqueous solutions, non-aqueous solutions in common solvents such as alcohols, or solutions in a liquid oil base.

According to some embodiments, the carrier of the composition of the present invention may include a release agent such as sustained release or delayed release carrier. In such embodiments, the carrier can be any material capable of sustained or delayed release of the active to provide a more efficient administration, e.g., resulting in less frequent and/or decreased dosage of the composition, improve ease of handling, and extend or delay effects on diseases, disorders, conditions, syndromes, and the like, being treated, prevented or promoted. Non-limiting examples of such carriers include liposomes, microsponges, microspheres, or microcapsules of natural and synthetic polymers and the like. Liposomes may be formed from a variety of phospholipids such as cholesterol, stearylamines or phosphatidylcholines.

The compositions of the present invention may be administered parenterally in the form of a sterile injectable aqueous or oleaginous suspension.

The sterile composition of the present invention may be a sterile solution or suspension in a nontoxic parenterally acceptable diluent or solvent. A solution generally is considered as a homogeneous mixture of two or more substances; it is frequently, though not necessarily, a liquid. In a solution, the molecules of the solute (or dissolved substance) are uniformly distributed among those of the solvent. A suspension is a dispersion (mixture) in which a finely-divided species is combined with another species, with the former being so finely divided and mixed that it doesn't rapidly settle out. In everyday life, the most common suspensions are those of solids in liquid water. Among the acceptable vehicles and solvents that may be employed are water, Ringer's solution, and isotonic sodium chloride (saline) solution. According to some embodiments, hypertonic solutions are employed. In addition, sterile, fixed oils are conventionally employed as a solvent or suspending medium. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran.

Additional compositions of the present invention can be readily prepared using technology which is known in the art such as described in Remington's Pharmaceutical Sciences, 18th or 19th editions, published by the Mack Publishing Company of Easton, Pa., which is incorporated herein by reference.

As used herein the terms “therapeutic amount,” “or “pharmaceutically effective amount” refer to the amount of the compositions of the invention that result in a therapeutic or beneficial effect following its administration to a subject. The therapeutic, or pharmaceutical effect can be curing, minimizing, preventing or ameliorating a disease or disorder, or may have any other pharmaceutical beneficial effect. The concentration of the substance is selected so as to exert its therapeutic or pharmaceutical effect, but low enough to avoid significant side effects within the scope and sound judgment of the physician. The effective amount of the composition may vary with the age and physical condition of the biological subject being treated, the severity of the condition, the duration of the treatment, the nature of concurrent therapy, the timing of the infusion, the specific composition or other active ingredient employed, the particular carrier utilized, and like factors.

A skilled artisan can determine a pharmaceutically effective amount of the inventive compositions by determining the dose in a dosage unit (meaning unit of use) that elicits a given intensity of effect, hereinafter referred to as the “unit dose.” The term “dose-intensity relationship” refers to the manner in which the intensity of effect in an individual recipient relates to dose. The intensity of effect generally designated is 50% of maximum intensity. The corresponding dose is called the 50% effective dose or individual ED50. The use of the term “individual” distinguishes the ED50 based on the intensity of effect as used herein from the median effective dose, also abbreviated ED50, determined from frequency of response data in a population. “Efficacy” as used herein refers to the property of the compositions of the present invention to achieve the desired response, and “maximum efficacy” refers to the maximum achievable effect. The amount of the chemotactic hematopoietic stem cell product in the pharmaceutical compositions of the present invention that will be effective in the treatment of a particular disorder or condition will depend on the nature of the disorder or condition, and can be determined by standard clinical techniques. (See, for example, Goodman and Gilman's THE PHARMACOLOGICAL BASIS OF THERAPEUTICS, Joel G. Harman, Lee E. Limbird, Eds.; McGraw Hill, N.Y., 2001; THE PHYSICIAN′S DESK REFERENCE, Medical Economics Company, Inc., Oradell, N.J., 1995; and DRUG FACTS AND COMPARISONS, FACTS AND COMPARISONS, INC., St. Louis, Mo., 1993). The precise dose to be employed in the formulations of the present invention also will depend on the route of administration and the seriousness of the disease or disorder, and should be decided according to the judgment of the practitioner and each subject's circumstances. It is envisioned that subjects may benefit from multiple administrations of the pharmaceutical composition of the present invention.

The therapeutically amount of the sterile isolated chemotactic hematopoietic stem cell product that is effective to restore heart function is at least 0.5×10⁶ potent SDF-1 mobile CD34+/CXCR-4+ cells having CXCR-4 mediated chemotactic activity per dosage unit for parenteral administration. The therapeutically amount of the sterile isolated chemotactic hematopoietic stem cell product that is effective to treat a traumatic brain or spinal cord injury will be similar on a per organ weight basis, depending on the organ weight, the type of artery or a branch, flow within the artery or branch, and region of the artery or branch where the product is administered. According to one embodiment, the therapeutically effective amount of the sterile isolated chemotactic hematopoietic stem cell product is at least 0.5×10⁶ potent SDF-1 mobile CD34+/CXCR-4+ cells having CXCR-4 mediated chemotactic activity per dosage unit for parenteral administration at the physician's discretion. According to some embodiments, the delivered volume is an amount that does not cause unwanted adverse effects, such as hydrocephalus, a condition characterized by an excessive accumulation of cerebrospinal fluid resulting in dilation of the cerebral ventricles and raised intracranial pressure.

According to another embodiment, the pharmaceutical compositions of the present invention can be administered by a combination therapy, wherein the pharmaceutical compositions can further include one or more compatible active ingredients which are aimed at providing the composition with another pharmaceutical effect in addition to that provided by the isolated chemotactic hematopoietic stem cell product of the present invention. “Compatible” as used herein means that the active ingredients of such a composition are capable of being combined with each other in such a manner so that there is no interaction that would substantially reduce the efficacy of each active ingredient or the composition under ordinary use conditions.

According to some embodiments, the hematopoietic stem cell mobilizing agent is a cytokine. According to some embodiments, the hematopoietic stem cell mobilizing agent is a colony stimulating factor. According to some such embodiments, the hematopoietic stem cell mobilizing agent comprises G-CSF, GM-CSF, or a pharmaceutically acceptable analog or derivative thereof. According to some embodiments, the hematopoietic stem cell mobilizing agent is a recombinant analog or derivative of a colony stimulating factor. According to some embodiments, the hematopoietic stem cell mobilizing agent is filgrastim. According to some embodiments, the hematopoietic stem cell mobilizing agent is mobilzil.

According to some embodiments, the combination therapy comprises administering to a subject in need thereof a pharmaceutical composition comprising a chemotactic hematopoietic stem cell product of the present invention combined with a second agent including, but not limited to, an anticoagulant agent, an anti-epileptic agent, an anti-inflammatory agent, and an antibiotic.

According to some embodiments, the composition of the present invention further comprises about 0.5% to about 5% albumin. According to some such embodiments, the minimum amount of albumin is about 0.5%, about 0.75%, about 1.0%, about 1.25%, about 1.5%, about 1.75%, about 2.0%, about 2.5%, about 2.75%, about 3.0%, about 3.5%, about 4.0%, about 4.5%, or about 5.0%, expressed as ml/100 cc volume of the composition. According to some other embodiments, the maximum amount of albumin in the compositions of the present invention is about 5.0%, about 4.75%, about 4.5%, about 4.25%, about 4.0%, about 3.75%, about 3.5%, about 3.25%, about 3.0%, about 2.75%, about 2.5%, about 2.25%, about 2.0%, about 1.75%, about 1.5%, about 1.25%, or about 1.0%, expressed as ml/100 cc volume of the composition. According to some embodiments, the albumin is human albumin. According to some embodiments the albumin is recombinant human albumin.

### II. Methods for Preparing the Pharmaceutical Composition

According to another aspect, the present invention provides a method of preparing the pharmaceutical composition comprising a chemotactic hematopoietic stem cell product for treating a vascular insufficiency following a traumatic brain or spinal cord injury. The method comprises the steps of:

(1) acquiring a chemotactic hematopoietic stem cell preparation comprising a population of CD34+ cells, which contains a subpopulation of potent SDF-1 mobile CD34+/CXCR-4+ cells that have CXCR-4-mediated chemotactic activity from the subject under sterile conditions by a chemotactic cell acquisition process;

(2) sterilely purifying the population of CD34+ cells containing the subpopulation of potent SDF-1 mobile CD34+/CXCR-4+ cells having CXCR-4-mediated chemotactic activity from the preparation;

(3) sterilely formulating the CD34+ cells in step (2) to form the chemotactic hematopoietic stem cell product;

(4) sterilely formulating the chemotactic hematopoietic stem cell product containing the population of CD34+ cells, which further contain a subpopulation of potent SDF-1 mobile CD34+/CXCR-4+ cells having CXCR-4-mediated chemotactic activity to form a pharmaceutical composition;

(5) assessing sterility of the pharmaceutical composition;

(6) releasing the sterile pharmaceutical composition as eligible for infusion into the subject;

(7) loading a therapeutically effective amount of the pharmaceutical composition into a delivery apparatus; and

(8) optionally transporting the delivery apparatus containing the therapeutically effective amount of the sterile pharmaceutical composition comprising the chemotactic hematopoietic stem cell product to a catheterization facility for infusion into the subject.

According to some embodiments, the nonexpanded, isolated population of autologous mononuclear cells can be acquired from the subject at any time. According to some embodiments, the nonexpanded, isolated population of autologous mononuclear cells is acquired early after a traumatic brain or spinal cord injury. According to some such embodiments, the nonexpanded, isolated population of autologous mononuclear cells is acquired 1 day, 2 days, 3 days, 4 days, 5 days, 6 days, 7 days, 8 days, 9 days, 10 days, 11 days, 12 days, 13 days, 14 days or more after the occurrence of a traumatic brain or spinal cord injury. According to some embodiments, the nonexpanded, isolated population of autologous mononuclear cells is acquired late after the occurrence of a traumatic brain or spinal cord injury. According to some such embodiments, the nonexpanded, isolated population of autologous mononuclear cells is acquired at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 60 days, at least 90 days, at least 120 days, at least 150 days, at least 180 days or more after the occurrence of a traumatic brain or spinal cord injury.

According to one embodiment, step (2) is initiated within about 12 hours to about 24 hours of completion of acquiring step (1). According to another embodiment, releasing step (7) proceeds only if the sterile formulated cell product is to be infused into the subject within about 48 hours to about 72 hours of completion of acquiring step (1). According to another embodiment, step (2) is initiated within about 12 hours to about 24 hours of completion of acquiring step (1), and releasing step (6) proceeds only if the sterile formulated cell product is to be infused into the subject within about 48 hours to about 72 hours of completion of acquiring step (1).

According to one embodiment, step (5), i.e., the step of assessing sterility of the pharmaceutical composition further comprises the steps of (i) centrifuging the chemotactic hematopoietic stem cell product comprising potent SDF-1 mobile CD34+/CXCR-4+ cells to form a cell pellet and a supernatant, the cell pellet comprising the potent SDF-1 mobile CD34+/CXCR-4+ cells; (ii) sterilely removing the supernatant without disturbing the cell pellet; and (iii) analyzing whether the supernatant is contaminated by a microbe thereby determining the sterility of the cell pellet.

According to one embodiment, in step (1), the chemotactic cell acquisition process is a mini-bone marrow harvest technique used to acquire a preparation of mononuclear cells comprising CD34+ cells, which further contain a subpopulation of potent SDF-1 mobile CD34+/CXCR-4+ cells from the bone marrow of the subject under sterile conditions. For the bone marrow harvest technique, step (a) of the method further comprises the steps: (i) aspirating the bone marrow from a left posterior iliac crest and a right posterior iliac crest of the subject using the harvesting syringes and a mini-bone marrow harvest technique to form harvested bone marrow; and (ii) infusing the harvested bone marrow into a collecting bag. According to one embodiment, the harvesting syringes in step (i) and the collecting bag in step (ii) contain a preservative free heparinized solution comprising 0.9% normal saline. According to one embodiment, step (a) of the method further comprises the steps: preloading harvesting syringes with heparin prior to harvesting bone marrow from a subject, prior to the aspirating step (i). The final concentration of heparin in the heparinized saline solution is about 20 units per ml to about 25 units per ml.

Optionally, according to one embodiment of the method, the harvested bone marrow is transported to a processing facility different from the facility from which the bone marrow was harvested. According to one embodiment, the method for transporting the harvested bone marrow to the processing facility comprises the steps (a) placing the harvested bone marrow in a collection bag; (b) placing the collection bag in a secondary bag; (c) placing the secondary bag containing the collection bag in a shipping container comprising an interior compartment containing frozen wet ice and at least one sheet of bubble wrap; (d) affixing a temperature tag monitor to the interior compartment of the shipping container; (e) sealing the shipping container; and (f) shipping the shipping container to the processing facility.

According to another embodiment, in step (1), the chemotactic cell acquisition process is a peripheral blood collection technique used to acquire a preparation of mononuclear cells comprising a population of CD34+ cells, which further contain a subpopulation of potent SDF-1 mobile CD34+/CXCR-4+ cells from the peripheral blood of the subject under sterile conditions. For the peripheral blood collection technique, step (a) of the method further comprises the steps: mobilizing the population of mononuclear cells comprising CD34+ cells, which further contain a subpopulation of potent SDF-1 mobile CD34+/CXCR-4+ cells, using a hematopoietic stem cell mobilizing agent. According to some embodiments, the hematopoietic stem cell mobilizing agent is a colony stimulating factor. According to some such embodiments, the hematopoietic stem cell mobilizing agent comprises G-CSF, GM-CSF, or a pharmaceutically acceptable analog or derivative thereof. According to some embodiments, the hematopoietic stem cell mobilizing agent is a recombinant analog or derivative of a colony stimulating factor. According to some embodiments, the hematopoietic stem cell mobilizing agent is filgrastim. According to some embodiments, the hematopoietic stem cell mobilizing agent is mobilzil.

### III. Methods for Treating Traumatic Brain or Spinal Cord Injury

According to another aspect, the described invention provides a method for treating a vascular insufficiency following a traumatic brain or spinal cord injury, the method comprising:

(a) administering to a subject in need thereof via a delivery device a therapeutic amount of a pharmaceutical composition comprising:

(i) a therapeutic amount of a sterile chemotactic hematopoietic stem cell product containing an isolated, nonexpanded population of autologous mononuclear cells comprising a population of CD34+ cells;
wherein the pharmaceutical composition is
formulated for administration parenterally;
characterized in that the isolated population of mononuclear cells comprising a population of CD34+ cells further contains a subpopulation of potent SDF-1 mobile CD34+/CXCR-4 cells that have CXCR-4-mediated chemotactic activity, such that the therapeutic amount comprises at least 0.5×10⁶ potent SDF-1-mobile CD34+CXCR-4+ cells that have CXCR-4-medicated chemotactic activity; and
further characterized as having the following properties for at least 24 hours following acquisition of the chemotactic hematopoietic stem cell product when tested in vitro after passage through a catheter:
   (1) at least 70% of the cells are CD34+ cells;
   (2) retains at least 2% of the CXCR-4-mediated chemotactic activity of the subpopulation of subpopulation of potent SDF-1 mobile CD34+/CXCR-4+ cells that have CXCR-4-mediated chemotactic activity measured prior to purification;
   (3) is at least 70% viable; and
   (4) is able to form hematopoietic colonies in vitro; and

(ii) a stabilizing amount of serum, which is effective to retain the CXCR-4-mediated chemotactic activity and hematopoietic colony forming activity of the population of SDF-1 mobile CD34+CXCR-4+ cells from acquisition to infusion; and

(b) monitoring the subject's cognitive and neurologic functions,

wherein the therapeutic amount of the sterile chemotactic hematopoietic stem cell product is effective to improve perfusion and to preserve existing nerve cells and their function in an area of ischemia in the injured brain or spinal cord parenchyma.

Without being limited by theory, according to some embodiments, the effect of the sterile chemotactic hematopoietic stem cell product is mediated by paracrine and anti-apoptotic effects of the sterile isolated chemotactic hematopoietic stem cell product.

According to some embodiments, the traumatic brain or spinal cord injury comprises an infarct area injury, including, but not limited to, apoptotic nerve cell loss in the infarct area; adverse remodeling after an acute cerebral infarction, when compared to controls; a progressive decline in cognitive function following the acute cerebral infarction; hypoperfusion of at least one ischemic peri-infarct zone; and a combination thereof.

According to some embodiments, the therapeutic amount of the composition is effective to improve microvascular blood flow in the infarct area, to decrease infarct mass, to increase perfusion of at least one ischemic peri-infarct zone of the brain or spinal cord tissue, or a combination thereof when compared to controls.

According to some embodiments, the traumatic brain injury comprises a closed head injury, a concussive head injury, or a penetrating head injury.

According to some embodiments, the traumatic brain injury comprises a severe traumatic brain injury that requires craniectomy, for example, an acute subdural hematoma or parenchymal hematoma, which increases intracranial pressure.

According to some embodiments, the method comprises an assessment of the subject's cognitive function. According to some such embodiments, the patient's cognitive function is evaluated by utilizing Glasgow Coma Scale (Teasdale, G. and Jennett, B., Lancet, 2(7872): 81-84, 1974, incorporated by reference herein in its entirety).

The Glasgow Coma Scale (GCS) is the most widely used scoring system used in quantifying the level of consciousness following traumatic brain injury. It is used because it is simple has a relatively high degree of reliability and correlates well with outcomes following severe brain injury. When applying the GCS, the final score is determined by adding the values of E+V+M (eye opening score+verbal response score+motor response score). This number helps medical practitioners categorize the possible levels for survival, with a lower number indicating a more severe injury and a poor diagnosis. Generally, brain injury is classified as follows:
Mild brain injury: a score of 13 to 15
Moderate brain injury: a score of 9 to 12 (This usually suggests that there was a loss of consciousness greater than 30 minutes)
Severe brain injury: a score of 3 to 8.

According to some embodiments, the traumatic brain injury is a mild or mild-to-moderate closed head injury (such as a cerebral concussion, a cerebral contusion, an epidural hematoma, a subdural hematoma, an intraventricular hemorrhage, and a diffuse axonal injury) that does not require craniectomy.

According to some embodiments, the subject experiences transient or subclinical effects, and the neural tissue of the injured brain or spinal cord contains an ischemic peri-infarct zone.

According to another embodiment, the traumatic brain injury produces repeated concussive injuries to the brain or spinal cord.

According to some other embodiments, the subject has an increased risk of developing chronic traumatic encephalopathy (CTE). According to some such embodiments, the chronic traumatic encephalopathy (CTE) is associated with progressive tauopathy (i.e., pathological aggregation of tau protein (microtubule-associated protein) in neurons) in the injured brain or spinal cord tissue.

According to some embodiments, the extent of the traumatic brain or spinal cord injury of the subject is evaluated by neuroimaging. Examples of neuroimaging include, but are not limited to, computed axial tomography (CT) or computed axial tomography (CAT), magnetic resonance imaging (MRI), functional magnetic resonance imaging (fMRI), magnetoencephalography (MEG), positron emission tomography (PET), and single-photon emission computed tomography (SPECT).

Computed tomography (CT) scanning builds up a picture of the brain based on the differential absorption of X-rays. During a CT scan the subject lies on a table that slides in and out of a hollow, cylindrical apparatus. An x-ray source rides on a ring around the inside of the tube, with its beam aimed at the subjects head. After passing through the head, the beam is sampled by one of the many detectors that line the machine's circumference. Images made using x-rays depend on the absorption of the beam by the tissue it passes through. Bone and hard tissue absorb x-rays well, air and water absorb very little and soft tissue is somewhere in between. Thus, CT scans reveal the gross features of the brain but do not resolve its structure well.

Magnetic resonance imaging (MRI) uses magnetic fields and radio waves to produce high quality two- or three-dimensional images of brain structures without use of ionizing radiation (X-rays) or radioactive tracers.

Functional magnetic resonance imaging, or fMRI, is a technique for measuring brain activity. It works by detecting the changes in blood oxygenation and flow that occur in response to neural activity—when a brain area is more active it consumes more oxygen and to meet this increased demand blood flow increases to the active area. fMRI can be used to produce activation maps showing which parts of the brain are involved in a particular mental process.

Magnetoencephalography (MEG) is an imaging technique used to measure the magnetic fields produced by electrical activity in the brain via extremely sensitive devices known as SQUIDs. There are many uses for the MEG, including assisting surgeons in localizing pathology, assisting researchers in determining the function of various parts of the brain, neurofeedback, and others.

Positron Emission Tomography (PET) uses trace amounts of short-lived radioactive material to map functional processes in the brain. When the material undergoes radioactive decay a positron is emitted, which can be picked up be the detector. Areas of high radioactivity are associated with brain activity.

Single-Photon Emission Computed Tomography (SPECT) is similar to PET and uses gamma ray-emitting radioisotopes and a gamma camera to record data that a computer uses to construct two- or three-dimensional images of active brain regions. SPECT relies on an injection of radioactive tracer, or “SPECT agent,” which is rapidly taken up by the brain but does not redistribute. Uptake of SPECT agent is nearly 100% complete within 30 to 60 seconds, reflecting cerebral blood flow (CBF) at the time of injection. SPECT provides a “snapshot” of cerebral blood flow since scans can be acquired after seizure termination (so long as the radioactive tracer was injected at the time of the seizure).

According to another embodiment, the pharmaceutical composition further comprises a therapeutically effective amount of at least one compatible therapeutic agent.

According to another embodiment, the therapeutic amount of the compatible agent is capable of promoting the function of existing nerve cells to compensate for loss of function due to neuronal death.

According to another embodiment, the compatible therapeutic agent comprises a vasoactive agent, an anti-coagulant agent, an anti-platelet agent, an anti-hypercholesterolemic agent, or a combination thereof.

According to another embodiment, the anticoagulant agent is selected from the group consisting of a coumarin, heparin, an inhibitor of Factor Xa, batroxobin, hementin, and a combination thereof.

According to some embodiments, the compatible therapeutic agent comprises a cytokine, a placental growth factor, granulocyte colony-stimulating factor, macrophage colony-stimulating factor, a vascular endothelial growth factor, neuregulin 1, tumor necrosis factor-like weak inducer of apoptosis, or a combination thereof.

According to another embodiment, the cytokine is at least one selected from the group consisting of vascular endothelial growth factor (VEGF), placental growth factor (PIGF), granulocyte colony-stimulating factor (G-CSF), and macrophage colony-stimulating factor (M-CSF).

According to another embodiment, the vascular endothelial growth factor is selected from the group consisting of VEGF-A, VEGF-B, VEGF-C, and VEGF-D.

According to one embodiment, the compatible therapeutic agent is Neuregulin-1 (NRG)1. Neuregulin-1 (NRG1) is an agonist for receptor tyrosine kinases of the epidermal growth factor receptor family, consisting of ErbB1, 2, 3, and 4. (Fuller, S J, et al., J. Mol. Cell. Cariol. 44: 831-54 (2008). Binding of NRG1 to Erb4 increases its kinase activity and leads to heterodimerization with erbB2 or homodimerization with ErbB4 and stimulation of intracellular signal transduction pathways. Id. NFRG1 receptor subunits ErbB2 and ErbB4 also are expressed in differentiated cardiomyocytes. Id. Previous studies showed that neuregulins are mitogenic to neuron-restricted progenitor cells and play important roles during central nervous system (CNS) neurogenesis (Liu, Y. et al., Dev Biol, 283(2): 437-445).

According to another embodiment, the delivery device comprises an anticoagulant agent. According to some embodiments, the delivery device is coated with an anticoagulant agent that prevents clotting of blood. Examples of such delivery devices include, but are not limited to, a heparin coated polyurethane catheter. According to some other embodiments, the delivery device is made of a material that intrinsically acts as an anticoagulant agent.

According to some embodiments, the therapeutic amount of the pharmaceutical composition is administered by direct injection or infusion into the traumatic penumbra of the injured brain or spinal cord tissue.

According to some such embodiments, the therapeutic amount is administered via stereotactically-guided injection. According to another embodiment the therapeutic amount is administered via impedance-guided injection.

According to some embodiments, the pharmaceutical composition is administered via intravascular delivery. According to some such embodiments, the pharmaceutical composition is injected or infused into an artery or a branch thereof. According to one embodiment, the artery is a carotid artery. According to another embodiment, the artery is a cerebral artery or a branch thereof. According to another embodiment, the cerebral artery includes, but is not limited to, a middle cerebral artery or a branch thereof, an anterior cerebral artery or a branch thereof, a posterior cerebral artery or a branch thereof, a basilar artery or a branch thereof, or a posterior cerebral artery or a branch thereof. According to another embodiment, the artery is a femoral artery or a branch thereof. According to another embodiment, the artery is a brachial artery or a branch thereof.

According to some embodiments, a stent is inserted into the artery or a branch thereof prior to delivering the pharmaceutical composition in order to provide an access for the chemotactic hematopoietic stem cell product comprising potent SDF-1 mobile CD34+/CXCR-4+ cells to the injured brain or spinal parenchyma. According to some embodiments, the stent is inserted into the artery or a branch thereof following an angioplasty procedure. Angioplasty and stenting are usually done through a small incision or puncture in the skin, called the access site. A catheter then is inserted through the access site and guided through the blood vessel to the site of occlusion. The tip of the catheter carries the angioplasty balloon or stent. According to some other embodiments, a stent is inserted into the artery or a branch thereof following endarterectomy. Endarterectomy is a surgical procedure that removes plaque from an artery that has become narrowed or blocked. According to some other embodiments, the endarterectomy is combined with anti-platelet therapy.

According to another embodiment, the intravascular administration utilizes the stop-flow technique. According to another embodiment, the intravascular administration utilizes a flow-directed transport without stop flow. According to another embodiment, the intravascular administration utilizes a catheter that permits injection across the vasculature.

According to some embodiments, the delivery device used to deliver the pharmaceutical composition of the present invention to a subject in need thereof comprises an infusion syringe and a catheter.

According to some other embodiments, the delivery device is an intravascular device comprising (a) an infusion device attached to a sterile four-way stopcock containing the pharmaceutical composition comprising the chemotactic hematopoietic stem cell product; (b) a flushing device attached to the sterile four-way stopcock, the flushing device containing a flushing solution, and (c) a catheter attached to the delivery apparatus by the sterile four-way stopcock.

According to another embodiment, the infusion device is a syringe made of any suitable material.

According to some embodiments, the body and handle of suitable four way stopcocks may be made of the same or a different material as the syringe. Examples of suitable four-way stopcocks includes, without limitation, a stopcock having a polycarbonate body/polycarbonate handle, a stopcock having a polyethylene body/polyethylene handle, a stopcock having a polycarbonate body/polyethylene handle, or a disposable stopcock.

According to another embodiment, a device is further attached to the stopcock to regulate the pressure exerted on the delivered solution. According to another embodiment an integral flush device or syringe is attached to the stopcock.

The viability and potential efficacy of the chemotactic hematopoietic stem cell product of the present invention comprising potent SDF-1 mobile CD34+/CXCR-4+ cells depends on the cells maintaining their potency as they pass through a catheter. The catheter used in the methods of the present invention has an internal diameter of at least 0.36 mm. Any type of catheter having an internal diameter of at least 0.36 mm may be effective in delivering the pharmaceutical compositions of the present invention. For example, a flow control catheter, which slows drainage of blood through the coronary artery vasculature allows the cells time to transit through the blood vessel wall and into the tissue.

According to some embodiments, the catheter is a flow control catheter.

According to some embodiments, the catheter is a balloon catheter. The term “balloon catheter” refers to a type of “soft” thin flexible tube having an inflatable “balloon” at its tip which is used during a catheterization procedure to enlarge a narrow opening or passage within the body. The deflated balloon catheter is positioned, inflated to perform the necessary procedure, and deflated again to be removed. For example, without limitation, the following balloon dilatation catheters available from Cordis, Boston Scientific, Medtronic and Guidant having an internal diameter of about 0.36 mm have been validated (see Table 1).

TABLE 1
Balloon catheter validated for infusion of selected CD34+ cells through
the IRA

| | Name and Model | | Lumen Internal |
|---|---|---|---|
| Manufacturer | No. | Balloon Dimensions | Diameter |
| Cordis | Raptor OTW 579-130 | 15 mm × 3.0 mm | 0.36 mm (0.14 in.) |
| Boston Scientific | OTW Maverick | 15 mm × 3.0 mm | 0.36 mm (0.14 in.) |
| | 20620-1530 | | |
| Medtronic | OTW Sprinter SPR | 15 mm × 3.0 mm | 0.36 mm (0.14 in.) |
| | 3015W | | |
| Guidant | Voyager OTW | 15 mm × 3.0 mm | 0.36 mm (0.14 in.) |
| | 1009443-15 | | |

In addition, catheters have been described having a fluid delivery port adjacent the balloon such that the balloon may be inflated against a vessel wall to isolate the delivery site from hemodynamics opposite the balloon from the port, which may be located distally of the balloon. Additionally, balloon catheters have been disclosed having lumens ending in side ports disposed proximally to the balloon catheter; these balloon catheters generally may be referred to as “balloon/delivery” catheters, although particular references may use different descriptors. See, e.g., U.S. Pat. No. 5,415,636 to Forman, which is incorporated by reference herein in its entirety.

According to some embodiments, the pharmaceutical composition is administered multiple times at a plurality of infusion dates, or as needed in the judgment of a treating physician. According to one embodiment, the pharmaceutical composition is administered to the subject at a first infusion date. According to another embodiment, the pharmaceutical composition is administered to the subject at a first infusion date, and optionally, at a second infusion date. According to another embodiment, the pharmaceutical composition is administered to the subject at a first infusion date, and optionally at a second infusion date, and a third infusion date. According to another embodiment, the pharmaceutical composition is administered to the subject at a first infusion date, and optionally at a second infusion date, a third infusion date, and a fourth infusion date. According to another embodiment, the pharmaceutical composition is administered to the subject at a first infusion date, and optionally at a second infusion date, a third infusion date, a fourth infusion date, and a fifth infusion date. According to another embodiment, the pharmaceutical composition is administered to the subject at a first infusion date, and optionally at a second infusion date, a third infusion date, a fourth infusion date, a fifth infusion date, and a sixth infusion date. According to another embodiment, the pharmaceutical composition is administered to the subject at a first infusion date, and optionally at a second infusion date, a third infusion date, a fourth infusion date, a fifth infusion date, a sixth infusion date, and a seventh infusion date. According to another embodiment, the pharmaceutical composition is administered to the subject at a first infusion date, and optionally at a second infusion date, a third infusion date, a fourth infusion date, a fifth infusion date, a sixth infusion date, a seventh infusion date, and an eighth infusion date. According to another embodiment, the pharmaceutical composition is administered to the subject at a first infusion date, and optionally at a second infusion date, a third infusion date, a fourth infusion date, a fifth infusion date, a sixth infusion date, a seventh infusion date, an eighth infusion date, and a ninth infusion date. According to another embodiment, the pharmaceutical composition is administered to the subject at a first infusion date, and optionally at a second infusion date, a third infusion date, a fourth infusion date, a fifth infusion date, a sixth infusion date, a seventh infusion date, an eighth infusion date, a ninth infusion date, and a tenth infusion date. According to another embodiment, the pharmaceutical composition is administered to the subject at a first infusion date, and optionally at a second infusion date, a third infusion date, a fourth infusion date, a fifth infusion date, a sixth infusion date, a seventh infusion date, an eighth infusion date, a ninth infusion date, a tenth infusion date, and so on.

According to another embodiment, the first infusion date is a time after an inflammatory cytokine cascade production peaks after an occurrence of the traumatic brain or spinal cord injury. According to some embodiments, the first infusion date is at least about one day, at least about two days, at least about three days, at least about four days, at least about five days, at least about six days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or more after an occurrence of the traumatic brain or spinal cord injury.

According to some embodiments, the second infusion date is at least about one day, at least about two days, at least about three days, at least about four days, at least about five days, at least about six days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or more after an occurrence of the traumatic brain or spinal cord injury. According to some embodiments, the third infusion date is at least about one day, at least about two days, at least about three days, at least about four days, at least about five days, at least about six days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or more after an occurrence of the traumatic brain or spinal cord injury.

According to some embodiments, the fourth infusion date is at least about one day, at least about two days, at least about three days, at least about four days, at least about five days, at least about six days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or more after an occurrence of the traumatic brain or spinal cord injury.

According to some embodiments, the fifth infusion date is at least about one day, at least about two days, at least about three days, at least about four days, at least about five days, at least about six days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or more after an occurrence of the traumatic brain or spinal cord injury.

According to some embodiments, the sixth infusion date is at least about one day, at least about two days, at least about three days, at least about four days, at least about five days, at least about six days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or more after an occurrence of the traumatic brain or spinal cord injury.

According to some embodiments, the seventh infusion date is at least about one day, at least about two days, at least about three days, at least about four days, at least about five days, at least about six days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or more after an occurrence of the traumatic brain or spinal cord injury.

According to some embodiments, the eighth infusion date is at least about one day, at least about two days, at least about three days, at least about four days, at least about five days, at least about six days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or more after an occurrence of the traumatic brain or spinal cord injury.

According to some embodiments, the ninth infusion date is at least about one day, at least about two days, at least about three days, at least about four days, at least about five days, at least about six days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or more after an occurrence of the traumatic brain or spinal cord injury.

According to some embodiments, the tenth infusion date is at least about one day, at least about two days, at least about three days, at least about four days, at least about five days, at least about six days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or more after an occurrence of the traumatic brain or spinal cord injury.

According to some embodiments, the chemotactic hematopoietic stem cell product of the composition administered at the second, third, fourth, fifth, sixth, seventh, eighth, ninth and/or tenth infusion date is prepared from a frozen and thawed aliquot of a nonexpanded, isolated population of autologous mononuclear cells containing CD34+ cells.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and describe the methods and/or materials in connection with the publications are cited.

It must also be noted that as used herein and in the appended claims, the singular forms “a,” “and” and “the” include plural referents unless the context clearly dictates otherwise.

All technical and scientific terms used herein have the same meaning

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

It should be understood by those skilled in the art that various changes may be made and equivalents may be substituted without departing from the true spirit and scope of the Invention. In addition, many modifications may be made to adapt a particular situation, material, composition of matter, process, process step or steps, to the objective, spirit and scope of the described invention. All such modifications are intended to be within the scope of the claims appended hereto.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Acquisition Process for Acquiring Chemotactic Hematopoietic Stem Cell Product that can then be Enriched for CD34+ Cells

While it is contemplated that any acquisition process appropriate for acquiring the chemotactic hematopoietic stem cell product comprising potent CD34+ cells is within the scope of the present invention, the following example illustrates one such process referred to herein as a mini-bone marrow harvest technique.

### Preparation of Harvesting Syringes

According to one embodiment, prior to the bone marrow harvest, forty 10 cc syringes optionally loaded with about 2-ml of a preservative free heparinized saline solution (about 100 units/ml to about 125 units/ml, APP Cat. No. 42592B or equivalent) will be prepared under sterile conditions. According to one embodiment, heparin will be injected via a sterile port into each of two 100-ml bags of sterile 0.9% normal saline solution (“Normal Saline”, Hospira Cat. No. 7983-09 or equivalent) following removal of 10 cc to 12.5 cc of normal saline from each bag, resulting in a final heparin concentration of about 100 units/ml (U/ml) to about 125 units/ml (U/ml). 2-ml of the preservative free heparin solution (about 100 U/ml to about 125 U/ml) will be loaded under sterile conditions into each of the forty 10 cc syringes, which then are capped and placed into a sterile bag for transport to the harvesting site.

Subjects will be prepared for bone marrow harvest after written informed consent is obtained. Conscious sedation will be provided using standard institutional procedures and guidelines. Bone marrow harvest will be conducted under sterile conditions. The term “sterile conditions” as used herein includes proper scrubbing and gowning with a sterile mask and gloves worn by the harvesting attending and assistant. The harvesting procedure can be performed outside of an operating room as follows: after sterile prepping and draping, each iliac crest should be anaesthetized with a 1% lidocaine solution using a minimum of 10-ml for each crest. The area of anesthesia should be a circular area no less than 10 cm in diameter. The harvesting needle is inserted until the iliac crest is punctured. The cap and stylet is removed and 2-ml of marrow is harvested into the 10-ml harvesting syringe optionally containing 2-ml of the heparin solution. The syringe then is removed and placed on the sterile field. After re-inserting the stylet, the harvesting needle is advanced slightly and then rotated 90°. The stylet is then removed and an additional 2-ml of marrow is drawn into the harvesting syringe retrieved from the sterile field. This procedure is repeated two more times until the harvesting syringe contains 8-ml of marrow for a total of 10-ml of optionally heparinized marrow at a final heparin concentration of about 20 U/ml to about 25 U/ml. Finally, the full harvesting syringe is handed to the harvesting assistant and shaken and infused in the sterile collecting bag as described below. The harvesting physician then takes the other harvesting needle that had been flushed previously with the heparin solution and repeats this process.

The full harvesting syringe is infused in the sterile collecting bag as follows. The harvesting assistant is handed the full harvesting syringe and empties it in the 500-ml collecting bag though the sterile adaptor attached to the bag. Then the harvesting needle is flushed with the heparin solution in the flushing syringe and retuned to the sterile field.

The harvesting process is repeated on one iliac crest until about 19 syringes have been collected and emptied in the collecting bag. The same process is repeated on the other iliac crest until another about 19 syringes have been filled. A total of thirty-eight 8 ml aspirations from both iliac crest (ideally 19 from each iliac crest) will result in 302-ml of bone marrow harvested in a final volume of 380 ml at a heparin concentration of about 20 U/ml to about 25 U/ml.

The collecting bag is sealed by tying off the connecting tube three times and then clamped distal to the ties. The bag is appropriately labeled “Human Bone Marrow Collection” and the results of the harvesting procedure, including final volume collected and any procedure related complication, are recorded on the Mayo Clinical Risk Score (MCRS) case report form. The completed label is affixed to the bone marrow bag. The bag then is placed in a sterile carrying bag to be transported to the processing facility.

### Example 2

### Preparation of the Bone Marrow Product for Transportation

According to one embodiment, the harvested bone marrow is transported to the processing facility as follows. When the clinical site is prepared to ship the bone marrow preparation, 24-hour notice will be provided to the processing facility. The processing laboratory will make shipping arrangements at the earliest possible time for pickup for same day delivery to the processing laboratory. Immediately after the bone marrow is collected, the bone marrow product will be placed in the supplied shipping container. The shipping container contains two small blocks of frozen wet ice on the bottom and a sheet of bubble wrap on top of the wet ice. The bone marrow product is placed into a secondary bag and the secondary bag is placed on top of the bubble wrap. A temperature tag monitor (a sensor used to monitor the internal temperature) is affixed to the interior of the box. Another layer of bubble wrap then is placed on top of the product before the shipping container is sealed off.

### Example 3

### Selection of CD34+ Cells from the Harvested Bone Marrow Product

CD34+ cells will be isolated from the harvested bone marrow product. According to one embodiment, CD34+ cells will be isolated using the anti-CD34 monoclonal antibody (Mab), Dynabeads® M-450 Sheep anti-Mouse IgG, and PR34+™ Stem Cell Releasing Agent components of the Isolex 300i Magnetic Cell Selection System (Baxter Healthcare Corp. Cat. No. 4R9734) as described in U.S. Pat. Nos. 5,536,475, 5,035,994, 5,130,144, 4,965,204, 5,968,753, 6,017,719, 6,251,295, 5,980,887, 6,676,937, U.S. Published Application No. 2003/0232050, and the Isolex 300i Package Insert, each of which is incorporated herein by reference. This operating system has been adapted for isolation of CD34+ cells from bone marrow according to the present invention.

Upon arrival at the processing laboratory, the harvested bone marrow product (in the collecting bag) is inspected immediately and the bag checked for any leakage. The collection should be free flowing with no apparent clumps and should not be hemolyzed. The collection will not be used if the integrity of the bag has been breached in any way.

The bone marrow product should be processed within about 12 hours to about 24 hours of inspection. A 300-ml or 400-ml transfer pack container is obtained, and a plasma transfer set is attached to the sampling port of the container. The bone marrow product is transferred from the collecting bag to the transfer pack container. The pooled bone marrow collection product is mixed thoroughly by inverting the container twenty (20) times.

The pooled bone marrow collection product then is sampled for analysis. According to one embodiment, a total volume of 2.0 ml of the product is removed and aliquoted as follows: 0.3 ml is used for a duplicate run of Complete Blood Count (CBC) using a hematology analyzer; 0.2-ml is dispensed into a 75×100-mm glass tube for the detection of Gram positive and Gram negative bacteria by Gram Stain (Gram Stain Kit, VWR, Cat. NO. BB231401); as a sterility check, 0.6-ml is dispensed into a Tryptic Soy Broth (TSB) (VWR, Cat. No. 29446-184) bottle for aerobic bacteria growth assay, 0.6-ml is dispensed into a Fluid Thioglycollate Media (FTM) (VWR Cat. #29446-138) bottle for anaerobic bacteria growth assay, and 0.3-ml is used in flow analysis for CD34+ cell enumeration and cell viability.

The collection is weighed on an electronic scale, and the appropriate tare weight of the collection bag recorded. The relationship of the volume of the bone marrow product to the weight of the product can be expressed as

Volume (ml)=[Weight (gm) of product−Tare weight of bag (gm)]÷1.06 (gm/ml) (Formula 1)

The number of Total Nucleated Cells (TNC) in the bone marrow product is calculated using the white blood cell (WBC) count obtained from the CBC according to the following relationship:

TNC=WBC/μl×1000×Product volume (ml) (Formula 2)

The number of CD34+ cells in the bone marrow product is calculated from the following relationship:

Total CD34+ cells in the bone marrow product=Number of CD34+ cell/μl×1,000×Product volume (ml) (Formula 3)

The Red Blood Cell (RBC) volume of the bone marrow collection product is calculated from the following relationship:

RBC volume (ml)=Product volume (ml)×Hematocrit(%)/100 (Formula 4),

If the collection contains more than 20 ml of RBC, red blood cell depletion is required. RBCs are depleted by centrifugation. Centrifugation at 1000×g for 20 minutes at ambient temperature is performed to separate the buffy coat from the RBCs. The term “buffy coat” refers to a thin grayish white fraction of a blood sample that contains most of the white blood cells (leukocytes). Immediately after centrifugation, a 60 ml syringe is connected to the bottom of the centrifugation bag and the RBCs are removed. More than one syringe may be needed to collect all the packed RBC. The RBC depleted bone marrow product then is washed to remove fat contents.

A 1-ml syringe is used to remove 0.3-ml of the RBC-depleted bone marrow cell product through the transfer set attached to the product bag and a CBC performed. The TNC of the RBC depleted bone marrow product is determined from the relationship:

Total TNC of the RBC depleted product=WBC/μl of RBC depleted product×1000×180-ml (Formula 5)

The TNC recovery of the RBC depleted product, which must be at least 80% of the original product count, is calculated from the relationship:

TNC recovery=TNC of the RBC depleted product÷TNC of the unprocessed product×100% (Formula 6)

The total RBC volume is calculated as described supra; the RBC volume in the RBC depleted product should be less than <20-ml.

According to one embodiment of the present invention, the Isolex® 300i system is used to process the RBC-depleted product or the bone marrow product whose RBC volume is <20 ml according to the following processing steps:
(i) The bone marrow is washed automatically to remove platelets;
(ii) CD34 positive (CD34+) cells are labeled specifically for selection by incubation with the Isolex 300i CD34 monoclonal antibody (Mab);
(iii) Unbound reagent is removed by washing the cell suspension with buffer solution;
(iv) Sensitized CD34+ cells (meaning CD34+ cells labeled with CD34 Mab) are captured by Dynabeads® M-450 Sheep anti-Mouse IgG;
(v) A selection column is used to separate the magnetically-labeled Dynabeads having captured CD34+ cells from unwanted cells, which are washed through the selection column and collected in the Negative Fraction Bag; and
(vi) PR34+ Stem Cell Releasing Agent releases CD34+ cells from the column, and the CD34+ cells are collected in the End Product Bag. The system performs several washing steps, disposing most of the liquid into the Buffer Waste Bag.

The Isolex® selected CD34+ fraction is assayed as follows to determine WBC and CD34+ cell yields. The volume of the CD34 Positive Fraction is determined by mixing the cells in the End Product Bag; the bag is gently massaged by hand to ensure even cell distribution. A transfer set is inserted into the sampling port of the End Product Bag and a 60-ml syringe attached. The cell suspension is withdrawn into the syringe (maximum 50-ml at a time) in order to measure the total volume.

A 3-ml or 5-ml syringe is used to remove a 2.0-ml sample from the End Product Bag through the transfer set for quality control testing. The aliquoted volumes of the samples and the analyses performed on those samples are as previously described, i.e., CBC: 0.3-ml; Gram stain: 0.3-ml; CD34+ cell enumeration and cell viability: 0.2-ml.

The total TNC of the CD34 Positive Fraction is calculated from the relationship:

Total TNC of the Positive Fraction=WBC/μl of the Positive Fraction×1000×Volume of the Positive Fraction (Formula 7)

The TNC recovery of the Positive Fraction, which must be less than 5% of the original product count, is calculated from the following relationship:

TNC recovery=Total TNC of the Positive Fraction÷Total TNC of the unprocessed product×100% (Formula 8)

The total number of viable CD34+ cells in the Positive Fraction is determined from the following relationship:

Total CD34+ cells in the Positive Fraction=Number of CD34+ cells/μl of the final product×1,000×Final product volume (ml) (Formula 9)

The CD34+ cell recovery of the Positive Fraction is calculated from the following relationship:

CD34+ cell recovery=Total CD34+ cells of the Positive Fraction÷Total CD34+ cells of the unprocessed product×100% (Formula 10).

### Example 4

### Preparation of Selected CD34+ Cells for Infusion

Samples of the chemotactic hematopoietic stem cell product will be removed to be assayed for WBC count, by flow cytometry (for CD34+ cell enumeration and viability), Gram stain, and sterility.

CD34+ cells are characterized by flow cytometric analysis featuring CD34 bright and CD45dim fluorescence by double labeling with anti-CD34 and anti-CD45 antibodies (Beckman Coulter, PN IM3630). CD34+ cells and CD45+ cell viability is determined by excluding the dying cells which take up the intercalating DNA dye 7-aminoactinomycin D (7AAD). See Brocklebank A M, Sparrow R L. Cytometry. 2001; 46:254-261 (2001); Barnett D, et al. Br. J. Haematol. 106:1059-1062 (1999); Sutherland, et al., J Hematotherapy 5:213-226 (1996), and U.S. Pat. Nos. 4,520,110; 4,859,582; 5,055,556; European Patent No. 76.695; Canadian Patent No. 1,179,942 (PE, APC); U.S. Pat. No. 4,876,190 (PerCP); U.S. Pat. Nos. 5,268,486; 5,486,616; 5,569,587; 5,569,766; 5,627,027 (Cy); U.S. Pat. Nos. 4,714,680; 4,965,204; 5,035,994 (CD34); U.S. Pat. No. 5,776,709 (Lyse/no-wash method); U.S. Pat. Nos. 5,723,218 and 5,187,288 (TruCOUNT Tubes), the contents of each of which is incorporated by reference herein in its entirety.

Any flow cytometer or an equivalent device can be used for conducting analysis of CD34+ cell enumeration and viability. According to one embodiment, the processing laboratory employs a BD FACSCalibur™ flow cytometer and BD FACSComp™ software is used for instrument setup and monitoring. A template and a panel of legend labels are preinstalled for acquisition and analysis. Prior to use, the reagents, namely CD45FITC/CD34PE, Stem-Count Fluorospheres, Concentrated Ammonium Chloride Lysing Solution, and 7-AAD Viability Dye, are brought to ambient temperature. CD34+ cell controls are run as a positive control to affirm that the instrument is set up for analyzing CD34+ cells, and the results are compared with the manufacturer's pre-determined CD34 percent range.

The unprocessed bone marrow product and Isolex processed chemotactic hematopoietic stem cell products may be analyzed by many different procedures. For example, immediately upon receiving the sample, if the WBC count of the sample is greater than 2×10⁷ cells per ml, the sample is diluted with Sheath fluid to achieve a cell count of about 2×10⁷ WBC per ml. 100 μl of the diluted product is aliquoted into two 15×100 mm tubes. Using a micropipetter, 20 μl of CD45FITC/CD34 PE and 7-AAD viability dye reagent are added into each tube and the samples gently vortexed. The tubes are covered with aluminum foil and left at ambient temperature for 15 to 20 minutes. RBCs are lysed by adding 1.5 ml of 1× Lysing Solution to each tube, vortexing gently. The tubes are incubated for ten minutes at ambient temperature, protected from light. The samples are stored at about 2° C.-about 8° C. (i.e., on an ice bath) protected from light until data acquisition is performed. Data acquisition must be performed within one hour of adding the lysing buffer. Before data acquisition, Stem-Count Fluorospheres are resuspended by end-over-end rotation (10 times). 100 μl of Fluorospheres is added to each tube and gently vortexed taking care not to generate air bubbles. The absolute count of CD34+ cells in the product is calculated from the relationship:

$$Number of viable CD 34 \text{+} cells per µl of product = \frac{LCD 34 \times FAC}{F} \qquad ( Formula 11 )$$

where LCD34 is the averaged number of events for Live CD34+/All CD 45+; “FAC” is Fluorospheres Assayed Concentration; and F is the averaged number of Fluorosphere singlets counted.

The volume of CD34+ Positive Fraction is calculated to obtain the number of CD34+ cells required for the required dosing. The Required Positive Fraction Volume (ml) is defined as:

The Requested CD34+ cell dosage÷(Total CD34+ cells per μl in the Positive Fraction×1,000). (Formula 12)

An appropriate number of cells is dispensed into a 50 ml conical tube and centrifuged at 500×g for 10 minutes. The supernatant is removed using a 30 ml serological pipette and disposed of as waste while exercising care not to disperse the cell pellets at the bottom of the tubes during this process. The infusion solution (20 ml) is added into the CD34+ Cell Positive Fraction tube and the cells dispersed using a 10 ml serological pipette by repeat pipetting. The resuspended cells are centrifuged for 10 minutes at 500 g. A 30 ml serological pipette is used (without disturbing the cell pellet) to transfer the supernatant/infusion solution into a 50 ml conical tube with a label “Positive Fraction Supernatant” affixed. The tube containing the supernatant is vortexed to homogenize the solution. A 10 ml serological pipette is used to transfer 10 ml of the homogenized supernatant back to the CD34+ Cell Positive Fraction tube. The remaining 10 ml of suspension in the Supernatant tube will be used for sterility testing (5 ml each into a TSB (Trypticase Soy Broth) bottle and an FTM (Fluid Thioglycollate) bottle). The cells in the CD34+ Cell Positive Fraction are resuspended by slowly withdrawing and aspirating through a blunt end needle affixed to a 10 ml syringe (Infusion Syringe) several times. The cell suspension is withdrawn into the syringe, any air bubbles are aspirated off, and the blunt end needle removed. The infusion syringe is attached to the injection port of a 4-way stopcock.

The chemotactic hematopoietic stem cell product of the present invention will be released for infusion only if it meets the following criteria:

CD34+ cell purity of at least about 70%, 75%, 80%, 85%, 90% or 95%;

A negative Gram stain result for the selected positive fraction;

Endotoxin Levels: less than about 0.5 endotoxin units/ml;

Viable CD34+ cell yield of the “Chemotactic hematopoietic stem cell product” meets the required dosing as per the treatment cohort;

CD34+ cells are at least about 70%, 75%, 80%, 85%, 90% or 95% viable by 7-AAD;

USP sterility result for “Positive Fraction Supernatant”: negative (14 days later); and

Bone marrow CD34+ cell selection was initiated within about 12 hours to about 24 hours of completion of bone marrow harvest.

Sterility assessment on the stem cell product including gram staining and endotoxin will be performed prior to product release for infusion. USP sterility (bacterial and fungal) culture will be performed and the results will be reported to the principal investigator. In the event of a positive USP sterility result, the subject and attending physician on call will be notified immediately, provided with identification and sensitivity of the organism when available, and documentation of appropriate anti-microbial treatment and treatment outcome will be recorded by the investigative site and the sponsor.

After meeting these release criteria, the chemotactic hematopoietic stem cell product will be released for infusion and packaged for transportation to the catheterization facility. A sample also will be sent for in vitro testing. Product will be released only if CD34+ cell selection is initiated within 12 hours to about 24 hours of completion of bone marrow harvest and only if it is to be infused within about 48 hours to about 72 hours of completion of bone marrow harvest.

### Example 5

### Formulation of the Chemotactic Hematopoietic Stem Cell Product Comprising CD34+ Cells

The chemotactic hematopoietic stem cell product is formulated in 10-ml of saline (0.9% Sodium Chloride, Injection, USP, Hospira, Cat#7983-09) supplemented with 1% HSA (Human Albumin USP, Alpha, Cat. #521303) (“Infusion Solution”) and about 0.1% (v/v) to about 70% autologous serum. In addition, there may be some trace amount of materials (quantities not determined) in the chemotactic hematopoietic stem cell product that are used and left over during the product processing. These materials include: Dulbecco's Phosphate Buffered Saline-Ca++, Mg++Free (D-PBS) (Baxter, Cat. # EDR9865), Sodium Citrate (Baxter/Fenwal, Cat. #4B7867), Hetastarch (Abbott Laboratories, Cat. #0074-7248-03), IVIg (Gammagard®

Immune Globulin Intravenous, Baxter, Cat. #060384) and the reagents in the Isolex® 300i Stem Cell Reagent Kit (Baxter, Cat. #4R9734) including anti-CD34 monoclonal antibody, stem cell releasing agent and Sheep anti-mouse magnetic beads.

### Example 6

### Transporting Chemotactic Hematopoietic Stem Cell Product to the Administration Site

The chemotactic hematopoietic stem cell product that meets the release criteria will be loaded into a sterile 10 cc syringe in a Class 100 biological safety cabinet located within a controlled aseptic environment, e.g., at minimum, a Class 100,000 cell processing facility; class 10,000 is preferable, but not required. The chemotactic hematopoietic stem cell product will be suspended in 10-ml PBS supplemented with HSA and a stabilizing amount of serum (see below) and the container labeled in accordance with release criteria. The loaded infusion syringe will be attached to a four-way stopcock along with a flushing syringe, capped and have safety guards applied to prevent leakage. The delivery apparatus will be sealed in a double sterile bag and placed in a secure transportation box for transportation to the administration/catheterization facility. Following release of the chemotactic hematopoietic stem cell product and cohort assignment, the chemotactic hematopoietic stem cell product will be shipped to the administration site for direct infusion or intravascular administration.

Statistical Analysis

According to some embodiments, a paired design, where each subject serves as his or her own control, will be used. Differences between before and after treatment, per subject, will be analyzed for brain function. Linear regression analysis will be used to assess the significance of increased dosing levels. The null hypothesis is that the slope of the regression line (dosing level serving as the independent variable and the “after” minus the “before” difference serving as the dependant variable) is equal to zero. The power of rejecting a false null hypothesis is 0.68 at the 0.05 alpha level of significance for a high correlation of 0.5 between dosing and improvement in cognitive function. The 95% confidence interval about the slope of the regression line will be used to assess the medical significance of the increase in dosing level. If the slope of the regression line is not significantly different from zero but the intercept of the regression line is different from zero, then all treatment groups will be combined and a paired t-test will be performed to assess the overall treatment effectiveness. The null hypothesis is that the mean of the differences is equal to zero. A Wilcoxon signed-ranks test also will be performed as an additional test to determine the treatment effectiveness. This test is more powerful (rejecting a false null hypothesis) than a t-test if the observations are not normally distributed. The power of the t-test is 0.79 for rejecting a false null hypothesis at the alpha level of 0.05 and the treatment having a medium size effect (an effect large enough to be discernable by the naked eye). The medical significance of the treatment effect size will be determined by computing a 95% confidence interval about the mean of the differences (the true mean of the differences will lay in this interval in 95% of tested samples).

### Example 7

### Experimental Results of Preliminary Studies

A series of preliminary preclinical studies have been performed in an attempt to accomplish the following goals:
(1) Optimize the manufacturing process for the Mini bone-Marrow Harvest (MMH);
(2) Evaluate the stability of the inbound MMH product and the outbound hematopoietic cell product;
(3) Evaluate the internal diameter allowance and safety of the catheters;
(4) Evaluate the compatibility of the cell product with the catheters intended to be used in the study; and
(5) Evaluate the suitability of using the supernatant of the final hematopoietic cell product to represent the final hematopoietic cell product for stability testing.

### Study 1: Optimizing the Manufacturing Process for the Mini Bone-Marrow Harvest (MMH);

The effect of key manufacturing variables on the yield of viable CD34 cells from representative bone marrow products was evaluated. A total of six (6) volunteer donors over the age of 45 (based on a range of 45-57) and three under 30 years of age (based a range of 21-28) agreed to donate an average of 45 ml (based on a range of 31 ml-54 ml) bone marrow and provided written Informed Consent for the procedure. The marrow aspiration technique employed was identical to that to be performed for the clinical scale MMH (see Example 3, supra). As shown in Table 2, the cell counts of nucleated cell (NC) and CD34+ cells of Mini bone-Marrow Harvest (“MMH”) derived cells collected from volunteer donors appeared to be age related.

TABLE 2
Effect of donor age on nucleated cell yield of the MMH.

| | Donor age group | | | | | |
|---|---|---|---|---|---|---|
| | Over 45 (45-57) | | | Under 30 (23-28) | | |
| | Volume of | Viability | CD34 cells | Volume of | Viability | CD34 cells |
| Donor | MMH (ml) | (%) | (10⁵ per ml) | MMH (ml) | (%) | (10⁵ per ml) |
| 1 | 31.30 | 83.85 | 1.27 | 48.00 | 96.90 | 7.98 |
| 2 | 43.50 | 97.42 | 3.89 | 50.60 | 96.28 | 11.60 |
| 3 | 51.50 | 85.74 | 1.37 | 39.90 | 87.17 | 5.99 |
| 4 | 47.50 | 80.95 | 1.76 | — | — | — |
| 5 | 53.70 | 98.21 | 5.58 | — | — | — |
| 6 | 44.90 | 96.36 | 4.48 | — | — | — |
| Avg. | 45.40 | 90.42 | 3.06 | 46.17 | 93.45 | 8.52 |

The average cell count of the bone marrow products from older donors (N=6) was 28.4×10⁶ (based on a range of 15.8×10⁶−49.5×10⁶) nucleated cells per ml [“NC/ml”], with an average viability, as determined by 7-AAD dye exclusion and flow cytometry, of 90.42% (based on a range of 80.95%-98.21%) and CD34+ content of 3.06×10⁵/ml (based on a range of 1.27×10⁵/ml−5.58×10⁵/ml). In the younger subject group (N=3), the average cell count collected from marrow aspiration was 46.2×10⁶ NC/ml (based on a range of 39.9×10⁶ NC/ml−50.6×10⁶ NC/ml), with an average 7-AAD viability of 93.5% (based on a range of 87.17%-96.90%) and total CD34+ content of 8.5×10⁵/ml (based on a range of 5.99×10⁵ CD34+ cells/ml−11.60×10⁵ CD34+ cells/ml).

Red Cell Depletion and CD34 Selection

TABLE 3
CD34+ cell recovery after RBC depletion of MMH from older age
group (4557) donors.

| | Donor | | | | | |
|---|---|---|---|---|---|---|
| | 1 | 2 | 3 | 4 | 5 | Average |
| Method of RBC | Hetastarch | Buffy | Buffy | Buffy | Buffy | — |
| depletion | | coat | coat | coat | coat | |
| CD34+ cell % | 1.09 | 1.64 | 1.63 | 1.45 | 1.99 | 1.58 |
| in MMH: | | | | | | |
| Pre-RBC depletion | | | | | | |
| CD34+ cell % | 1.33 | 1.55 | 1.51 | 1.61 | 1.84 | 1.57 |
| in MMH: | | | | | | |
| Post-RBC depletion | | | | | | |
| CD34+ cell recovery | 65.68 | 92.36 | 80.66 | 78.79 | 81.67 | 79.83 |
| post RBC depletion | | | | | | |
| (%) | | | | | | |

As shown in Table 3, following red cell depletion of the MMH-derived bone marrow products collected from the older donors, an average of 79.83% (based on a range of 65.68%-92.36%) of the CD34 cells from the initial MMH was recovered. There was no significant difference between the initial CD34 cell purity (1.58%, based on a range of 1.09%-1.99%) and that following red cell depletion (1.57%, based on a range of 1.33%-1.84%).

TABLE 4
CD34+ cell recovery, purity, CXCR-4 migratory activity, viability and
hematopoietic CFU growth immediately after Isolex processing of MMH
from older age group (age 45-age 57) donors.

| | Donor | | | | | |
|---|---|---|---|---|---|---|
| | 1 | 2 | 3 | 4 | 5 | Average |
| Storage time (hours) at | 0 | 0 | 0 | 12 | 10.50 | — |
| 4° C.-8° C. | | | | | | |
| CD34+ cell recovery (%) | 32.36 | 29.09 | 15.31 | 43.60 | 40.20 | 32.11 |
| CD34+ cell purity (%} | 76.76 | 73.64 | 71.66 | 72.52 | 72.01 | 73.32 |
| CD34+ cell viability | 98.49 | 93.80 | 97.38 | 98.28 | 98.39 | 97.27 |
| CD34+ cell CXCR-4 | 22.10 | 2.60 | 22.00 | 19.90 | 19.70 | 17.26 |
| migratory activity (%) | | | | | | |
| Hematopoietic CFU/100 | 27.5 | 25.0 | 18.9 | 17.0 | 21.00 | 21.9 |
| CF34+ cells cultured | | | | | | |

As shown in Table 4, following CD34 selection using the Isolex system, which includes immunomagnetic Dynabeads® and anti-CD34 MAb, we recovered an average of 32.11% (based on a range of 15.31%-43.60%) of the CD34 cells with an average purity of 73.32% (based on a range of 71.66%-73.64%) and an average viability of 97.27% (based on a range of 93.80%-98.49%). In addition, these CD34+ cells displayed an average of 17.26% (based on a range of 2.60%-22.10%) CXCR-4 migratory ability immediately after selection and were capable of generating hematopoietic colonies (21.89 colonies/100 CD34+ cells plated (based on a range of 17.0 colonies/100 CD34+ cells plated−27.5 colonies/100 CD34+ cells plated) in MethoCult culture.

Study 2: Evaluation of the Stability of the Inbound Mini-Bone Marrow Harvest and of the Outbound Chemotactic Hematopoietic Cell Product

A series of experiments, using healthy volunteers, was performed in order to evaluate the stability of the inbound MMH and of the outbound chemotactic hematopoietic stem cell product of the present invention. Assessment of the functional viability of the inbound and outbound products was evaluated by cell viability (7-AAD), SDF-1/CXCR-4 mediated CD34+ cell migration, and the ability to form hematopoietic colonies in methylcellulose (CFU colony forming ability).

To evaluate the inbound product stability for shipping and logistic purposes and for coordination with clinical schedules, MMH products were stored at 4° C. to 8° C. as indicated. To evaluate the outbound product stability for shipping and logistic purposes, the chemotactic hematopoietic stem cell product comprising isolated CD34+ cells enriched following MMH was stored at 4° C. to 8° C. as indicated.

In preliminary studies, cells either were processed immediately or maintained at 4-8° C. for 12 hours prior to processing to evaluate the impact of shipping and logistic duration on the manufacture a suitable cell product for infusion. Despite the duration of storage prior to processing (inbound product expiration), the results did not vary significantly (data not shown).

In another series of experiments, cells were stored at about 4° C. to about 8° C. for 12 hours and about 24 hours prior to reassessment to simulate products infused at about 36 hours and at about 48 hours, respectively, following MMH.

TABLE 5
CD34+ cell viability, growth and CXCR-4 migratory activity
13-13.5 hours after Isolex processing of MMH.

| | Donor | | |
|---|---|---|---|
| | 1 | 2 | Average |
| CD34+ cell viability (%) | 97.59 | 96.90 | 97.24 |
| CD34+ cell CXCR-4 migratory activity (%) | 7.70 | 7.50 | 7.60 |
| Hematopoietic CFU/100 CD34+ cells cultured | 18.00 | 25.00 | 21.5 |

As shown in Table 5, the isolated CD34+ cells of the chemotactic hematopoietic stem cell product had an average viability of 97.24% (based on a range of 96.90%-97.59%) and average CXCR-4-mediated migratory capacity of 7.60% (based on a range of 7.50%-7.70%). As shown in Table 6, after storage for an average of 26.3 hours (based on a range of 26.0 h-26.5 h), these cells had an average viability of 96.81% (based on a range of 96.39%-97.22%) and an average CXCR-4-mediated migratory capacity of 4.75% (based on a range of 4.50%-5.00%). Further, the cells still maintained their ability to generate hematopoietic colonies in vitro.

TABLE 6
CD34+ cell viability, growth and CXCR-4 migratory activity
26.0-26.5 hours after Isolex processing of MMH.

| | Donor | | |
|---|---|---|---|
| | 1 | 2 | Average |
| CD34+ cell viability (%) | 97.22 | 96.39 | 96.81 |
| CD34+ cell CXCR-4 migratory activity (%) | 4.50 | 5.00 | 4.75 |
| Hematopoietic CFU/100 CD34+ cells cultured | 28.00 | 14.00 | 21.00 |

Thus, an average of 13.3 hours (based on a range of 13.0 h-13.5 h) after CD34+ cell selection, representing 26.0-26.5 hr post-MMH, the CD34+ cell population had an average viability of 97.24% (based on a range of 96.90%-97.59%), with average CXCR-4 mediated migratory capacity of 7.60% (based on a range of 7.50%-7.70%). At an average of 26.3 hours (based on a range of 26.0 h-26.5 h) following MMH, the average viability of the cells was 96.81% (based on a range of 96.39%-97.2%) and maintained an average CXCR-4-mediated migratory capacity of 4.75% (based on a range of 4.50%-5.00%).

Formulation of the composition of the present invention comprising this product occurred an average of 8 hours (8.63±1.80 N=4) after MMH collection, and infusion occurred within 24 hours of MMH.

TABLE 7
CD34+ cell viability as a function of time after MMH: 12-hour
in-dating and 48 hour outdating (all time points measured from
completion of MMH.)

| | CD34+ cell viability (%) | | | | |
|---|---|---|---|---|---|
| Time (h) after MMH | | | | | Average |
| (SD) | A | B | C | D | (SD) |
| | 98.22 | 97.13 | 97.60 | 99.00 | 97.99 |
| | | | | | (0.29) |
| 24 | 95.32 | 97.76 | — | — | 96.54 |
| | | | | | (1.73) |
| 33 | 91.92 | 96.32 | 95.90 | 80.00 | 91.04 |
| | | | | | (7.62) |

In a subsequent experiment, four (4) MMH products (A-D) were collected and stored at 4° C. for an average of 12.8 hours (based on a range of 12.5 h-13.0 h) before the CD34+ cells were isolated by the Isolex procedure. This group, representing the “12 hour in-date” group (meaning that the product was formulated within the in-date time of about 12 hours), was evaluated for functional viability out-date at “24 hours” (22.9 h±1.63, N=4), “33 hours” (33.38±1.11, N=2), and “48 hours” (48.33±0.82, N=4) post MMH harvest. The data, summarized in Tables 7-9, demonstrate that following MMH, the chemotactic hematopoietic stem cell product comprising enriched CD34+ cells maintains: (1) high viability (>90.0% average viability, Table 7); (2) 76.85% (±21.66) of their SDF-1/VEGF/CXCR-4 mediated migratory ability (Table 8); and 3) their ability to form hematopoietic colonies in vitro (Table 9), respectively.

Table 8 shows SDF-1/VEGF/CXCR-4 mediated CD34+ cell migration (% migrating CD34+ cells as a function of time after MMH: 12-hour in-dating and 48-hour outdating (all time points measured from completion of MMH). For the purposes of determining the impact of time post-MMH on the migratory ability of the CD34+ cells, time point “X” was considered the reference point, as this was determined to represent the earliest time point following MMH at which cells reasonably could be expected to be returned to the subject in a finished formulation. The remaining migratory activity at the following time points (Y=33 hours, Z=48 hours) was calculated as percent migratory ability remaining following the 24 hour (X) time point.

TABLE 8
SDF-1/VEGF/CXCR-4 mediated CD34+ cell migration
(% migrating CD34+ cells as a function of time
after MMH: 12-hour in-dating and 48-hour outdating
(all time points measured from completion of MMH).

| | Migrating CD34+ cells (%) | | | | |
|---|---|---|---|---|---|
| Time (h) | | | | | Average |
| after MMH | A | B | C | D | (SD) |
| 24 (X) | 20.00 | 18.50 | 21.50 | 36.00 | 24 |
| | | | | | (8.09) |
| % Remaining | 100.00 | 100.00 | 100.00 | 100.00 | 100.00 |
| | | | | | (0) |
| 33 (Y) | 21.80 | 10.50 | — | — | 16.15 |
| | | | | | (7.99) |
| *% Remaining | 109.00 | 56.76 | — | — | 82.88 |
| | | | | | (36.94) |
| 48 (Z) | 8.80 | 17.00 | 17.50 | 31.00 | 18.58 |
| | | | | | (9.19) |
| @% Remaining | 44.00 | 91.89 | 81.40 | 86.00 | 75.85 |
| | | | | | (21.66) |

*= (Y ÷ X) × 100%
@= (Z ÷ X) × 100%

Table 9 shows the number of colony forming units (CFU) per 100 viable CD34+ cells plated as a function of time after MMH: 12-hour in-dating and 48 hour-out-dating (all time points measured from completion of MMH.

TABLE 9

| | # of CFU per 100 viable CD34+ cells plated | | | | |
|---|---|---|---|---|---|
| Time (h) | | | | | Average |
| after MMH | A | B | C | D | (SD) |
| 24 | 13.00 | 30.00 | 37.00 | 39.00 | 29.75 |
| | | | | | (11.81) |
| 33 | 12.00 | 34.00 | — | — | 23.00 |
| | | | | | (15.56) |
| 48 | 15.00 | 30.00 | 20.00 | 8.00 | 28.25 |
| | | | | | (14.57) |

In an attempt to extend both the in-date and out-date stability parameters for the chemotactic hematopoietic stem cell product of the present invention comprising CD34+ cells from 12-hours (in-date) and from 48-hours (out-date) (12/48), respectively, to 24-hours (in-date) and 72-hours (outdate) (24/72), respectively, CD34 cells were purified about 12 hours after MMH harvest (12 hour in-date) and about 24 hours after MMH harvest (24 hour in-date) and analyzed for functional viability at about 48 hours and at about 72 hours total time from MMH to time of testing/anticipated infusion (48 hour out-date and 72 hour out-date, respectively). Specifically, the functional viability characteristics of two MMH/chemotactic hematopoietic stem cell products of the present invention were evaluated at 48 hours and 72 hours. The resulting data were further compared to the same indices derived at the previous 12/48 time points (Tables 7-9).

Tables 10-12 show that at 33 hours (based on 32.5±0.71, N=2), 48 hours (based on one data point at 49 hours), and at 72 hours (based on 72.5 h±0.71, N=2), the isolated CD34+ cells of the chemotactic hematopoietic stem cell product of the present invention maintain: (1) over 90% viability (Table 10), (2) 102.19±32.69% of their SDF-1/VEGF/CXCR-4 mediated migratory ability (Table 11), and (3) their ability to generate hematopoietic colonies in vitro (Table 12).

TABLE 10
CD34+ cell viability as a function of time
after MMH: 24-h in-dating and 72-h outdating
(all time points measured from completion of MMH)

| | CD34+ cell viability (%) | | |
|---|---|---|---|
| Time (h) | | | Average |
| after MMH | A | B | (SD) |
| 33 | 98.00 | 99.00 | 98.50 |
| | | | (0.71) |
| 48 | — | 97.00 | 97.00 |
| | | | (—) |
| 72 | 91.00 | 97.00 | 94.00 |
| | | | (4.24) |

TABLE 11
SDF-1/VEGF/CXCR-4 mediated CD34+ cell migration
(% population of migrated CD34+ cells as a function
of time after MMH): 24-h in-dating and 72-h outdating
(all time points measured from completion of MMH)

| | Migrating CD34+ cells (%) | | |
|---|---|---|---|
| Time (h) | | | Average |
| after MMH (SD) | A | B | (range) |
| 33 | 8.20 | 14.05 | 11.13 |
| | | | (2.93) |
| % Remaining | 100.00 | 100.00 | 100.00 |
| | | | (0.00) |
| 48 | — | 18.61 | 18.61 |
| | | | (—) |
| % Remaining | — | 132.46 | 132.46 |
| | | | (—) |
| 72 | 5.70 | 18.95 | 12.33 |
| | | | (6.63) |
| % Remaining | 69.51 | 134.88 | 102.19 |
| | | | (32.69) |

The % remaining ratios in Table 11 were determined as in Table 8 above.

TABLE 12
Number of CFU per 100 viable CD34+ cells plated as
a function of time after MMH: 24-h in-dating and 72-h outdating
(all time points measured from completion of MMH)

| | # of CFU per 100 viable CD34+ cells plated | | |
|---|---|---|---|
| Time (h) | | | Average |
| after MMH (SD) | A | B | (range) |
| 33 | 26.00 | 28.50 | 22.25 |
| | | | (1.25) |
| 48 | — | 16.80 | 16.80 |
| | | | (—) |
| 72 | 14.50 | 27.50 | 21.00 |
| | | | (6.5) |

Further evaluation of the functional viability parameters of the chemotactic hematopoietic stem cell product comprising isolated CD34+ cells of the present invention (“clinical product”) at 8 hours (8.6 h±1.80, N=4), 12 hours (12.87 h±1.92, N=4), 32 hours (one time point at 33.5 h), 48 hours (47.50 h±2.5, N=2), and 72 hours (71.5 h±0.50, N=2) after MMH shows that after 72 hours, the product retains its 1) viability (Table 13), 2) SDF-1/VEGF/CXCR-4 mediated migratory ability (Table 14), and 3) ability to form hematopoietic colonies in vitro (Table 15), equivalent to the 24-hour time point.

TABLE 13
Clinical Product Experience: CD34+ cell
viability as a function of time after MMH.

| | CD34+ cell viability (%) | | | | |
|---|---|---|---|---|---|
| Time (h) | | | | | Average |
| after MMH | A | B | C | D | (SD) |
| 8 | 98.30 | 99.08 | 90.00 | 96.45 | 95.96 |
| | | | | | (4.12) |
| 12 | 98.89 | 96.96 | 99.00 | 99.43 | 98.57 |
| | | | | | (1.10) |
| 33 | — | 93.42 | — | — | 93.42 |
| 48 | — | 93.15 | 91.58 | — | 92.37 |
| | | | | | (1.11) |
| 72 | — | 91.25 | 89.25 | — | 90.30 |
| | | | | | (1.48) |

TABLE 14
Clinical Product Experience: SDF-1/VEGF/CXCR-4 mediated
CD34+ cell migration (% migrating CD34+ cells
as a function of time after MMH)

| | Migrating CD34+ cells (%) | | | | |
|---|---|---|---|---|---|
| Time (h) | | | | | Average |
| after MMH | A | B | C | D | (SD) |
| 12 (X) | 14.31 | 13.08 | 9.74 | 31.73 | 17.97 |
| | | | | | (11.34) |
| % Remaning | 100.0 | 100.0 | 100.0 | 100.0 | 100.0 |
| | | | | | (0) |
| 33 (Y) | — | 6.17 | — | — | 6.17 |
| *% Remaining | — | 47.17 | — | — | 47.17 |
| 48 (Y) | — | 4.88 | 8.21 | — | 6.55 |
| | | | | | (2.35) |
| *% Remaining | — | 37.30 | 84.29 | — | 60.79 |
| | | | | | (23.49) |
| 72 (Y) | — | 3.7 | 6.6 | — | 5.15 |
| | | | | | (2.05) |
| *% Remaining | — | 28.29 | 21.19 | — | 24.74 |
| | | | | | (3.55) |

*= (Y ÷ X) × 100%
All remaining ratios were calculated as in Table 8 above.

TABLE 15
Clinical Product Experience: # of CFU per 100 viable CD34+
cells plated as a function of time after MMH

| | # of CFU per 100 viable CD34+ cells plated | | | | |
|---|---|---|---|---|---|
| Time (h) | | | | | Average |
| after MMH | A | B | C | D | (SD) |
| 12. | 98.14 | 33.30 | 24.00 | 22.50 | 44.49 |
| | | | | | (36.09) |
| 33 | — | 16.50 | — | — | 16.5 |
| 48 | — | 19.56 | 20.50 | — | 20.03 |
| | | | | | (0.66) |
| 72 | — | 20.45 | 21.19 | — | 20.82 |
| | | | | | (1.10) |

Based on these data, extension of the in-dating to 24 hours (from 12-hours) and the out-dating to 72 hours (from 48 hours) for the CD34+ cell clinical product of the present invention is justified.

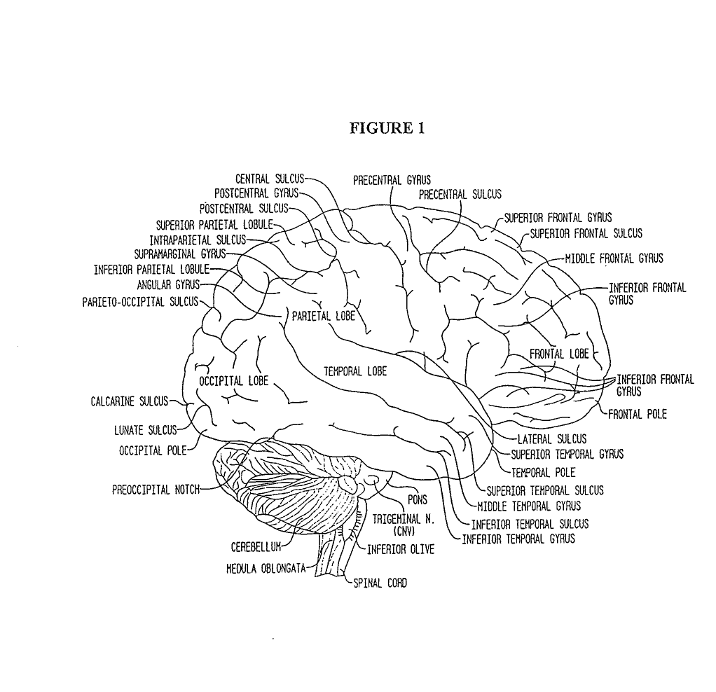

FIG. 1 indicates the equivalence of the functional viability of the chemotactic hematopoietic cell product of the present invention at 72 hours to the same indices evaluated at 48 hours.

Study 3: Catheter Safety

The viability and potential efficacy of the chemotactic hematopoietic stem cell product of the present invention comprising potent CD34+ cells depends on the cells maintaining their potency as they pass through a catheter. The catheter used in the methods of the present invention has an internal diameter of at least 0.36 mm. Any type of catheter having an internal diameter of at least 0.36 mm may be effective in delivering the pharmaceutical compositions of the present invention.

According to one embodiment, the catheter is a balloon catheter. Balloon catheter safety studies were conducted to determine whether high cell concentrations and repeated perfusions adversely affect cell viability, cell recovery or catheter integrity. Non-mobilized peripheral blood progenitors were used in order to obtain an adequate number of cells to perform the analysis. Catheters were assessed for infusion of the cell product of the present invention comprising selected CD34+ cells through the IRA. None of the 0.36 mm internal diameter catheters tested adversely affected CD34+ selected cell viability, growth in culture, or mobility in CXCR-4 assays.

TABLE 16
Viability of CD34+ cells before and after infusions through the catheters

| | | Viability (%) | | | | |
|---|---|---|---|---|---|---|
| Catheter | Condition | 1 | 2 | 3 | 4 | 5 |
| — | Pre-infusion | 81.45 | | | | |
| Raptor | After 1st infusion | 84.29 | 70.94 | 87.89 | 88.02 | 84.68 |
| | After 2nd infusion | 83.00 | 87.44 | 86.39 | 79.91 | 83.18 |
| Sprinter | After 1st infusion | 93.39 | 91.09 | 84.13 | 88.28 | 81.68 |
| | After 2nd infusion | 91.89 | 91.08 | 84.88 | 77.65 | 77.73 |
| Voyager | After 1st infusion | 94.21 | 86.21 | 83.08 | 77.53 | 69.68 |
| | After 2nd infusion | 88.03 | 84.71 | 79.27 | 78.11 | 76.80 |
| Maverick | After 1st infusion | 90.00 | 89.76 | 90.79 | 85.49 | 81.31 |
| | After 2nd infusion | 90.94 | 87.38 | 81.98 | 80.09 | 85.47 |

As shown in Table 16, in all catheters tested, average CD34+ cell viability was at or above 70% following passage through the catheters.

To demonstrate that infusion of the CD34+ cell product does not pose any safety breach of the catheter used and that a significant percentage of cell product does not adhere to the interior walls of the catheter, catheters were challenged with repeat infusions of a CD34+ cell product having a considerably higher cell concentration than that used clinically. Four brands of catheters (Sprinter, Voyager, Maverick and Raptor) were evaluated using 5 catheters of each type. Non-mobilized apheresis products were used in order to obtain an adequate number of cells to perform the analysis. A cell concentration greater than three times that planned as treatment doses for the trial, i.e., 160×10⁶ nucleated cells containing CD34+ cells in 10 ml of infusion solution, was passed twice through each catheter. The average CD34+ cell recovery was 100.59% (based on a range of 76.99% to 228.70%) following passage through the catheters.

All twenty catheters were tested for integrity using a methylene blue dye leak test after two perfusions with the nucleated cells. There was no evidence of leakage and the contact points and catheter tips were normal upon inspection.

As shown in Table 17a and 17b, the effect on the cells of their perfusion through a catheter appears to be independent of catheter model and make among those catheters tested and was independent of the amount of time the cells were stored either prior to processing and/or after CD34+ cell selection and prior to perfusion, resulting in a final formulation containing an average recovery of 96.0% (range 80.8%-102.2%) of the CD34+ cells (Table 17b) and 86.36% of the CD45+ cells perfused through the catheter. Further, the average viability of the cells was 96.5% (range 92.5%-98.6%, N=16); the cells maintained both CXCR-4 migratory capacity (data not shown) and their ability to form hematopoietic colonies in methylcellulose (average 25.8 CFU/100 cells seeded (range 21.0%-30.5%).

TABLE 17a
CD45 cell recovery and viability after being infused through the catheters

| | | 1 | | 2 | | 3 | | 4 | | 5 | | Average | |
|---|---|---|---|---|---|---|---|---|---|---|---|---|---|
| | Con- | | R'd | | R'd | | R'd | | R'd | | R'd | | R'd |
| Catheter | dition | Recovery | viab | Recovery | viab | Recovery | viab | Recovery | viab | Recovery | viab | Recovery | viab |
| Raptor | After^{1st} | 69.68% | −1.35% | 78.67% | 2.08% | 72.14% | −4.55% | 80.54% | 1.83% | 73.21% | −2.13% | 74.85% | −0.82% |
| | infusion | | | | | | | | | | | (30.83%) | (2.53%) |
| | After^{2nd} | 97.91% | −8.55% | 81.84% | −4.76% | 142.98% | 3.28% | 107.82% | −8.48% | 94.08% | 0.08% | 104.93% | −3.69% |
| | infusion | | | | | | | | | | | (47.60%) | (4.94%) |
| Sprinter | After^{1st} | 76.74% | −0.60% | 68.56% | 4.01% | 72.63% | 5.29% | 73.61% | 6.06% | 66.83% | 8.31% | 71.67% | 4.61% |
| | infusion | | | | | | | | | | | (29.48%) | (3.51%) |
| | After^{2nd} | 78.82% | 2.86% | 85.40% | 0.98% | 90.29% | −1.02% | 82.22% | 6.50% | 91.61% | 0.00% | 85.67% | 1.86% |
| | infusion | | | | | | | | | | | (35.30%) | (2.76%) |
| Voyager | After^{1st} | 87.38% | 1.58% | 83.93% | −0.36% | 103.58% | 0.93% | 95.82% | 4.52% | 131.55% | −4.39% | 100.45 | 0.46% |
| | infusion | | | | | | | | | | | (44.39%) | (2.91%) |
| | After | 82.70% | 7.01% | 69.34% | 15.90% | 69.54% | 10.40% | 89.04% | 0.27% | 69.03% | 7.50% | 75.93% | 8.22% |
| | 2nd | | | | | | | | | | | (32.11%) | (6.09%) |
| | infusion | | | | | | | | | | | | |
| Maverick | After^{1st} | 73.97% | 1.58% | 87.01% | 0.42% | 78.31% | 0.69% | 75.53% | 2.61% | 77.22% | 2.95% | 78.41% | 1.65% |
| | infusion | | | | | | | | | | | (32.33%) | (1.21%) |
| | After^{2nd} | 152.35% | −5.06% | 73.44% | 2.78% | 80.85% | −3.92% | 97.10% | −2.97% | 91.11% | −2.07% | 98.97% | −2.25% |
| | infusion | | | | | | | | | | | (49.11%) | (2.85%) |

| | | |
|---|---|---|
| Average of all catheters: | 86.36% | 1.26% |

^{a}Recovery of CD45+ cells = (# of CD45 cells after infusion ÷ # of CD45 before infusion) × 100%
^{b}Reduction of CD45+ cell viability = [1 − (CD45+ cell viability % after infusion ÷ CD45+ cell viability % before infusion)] × 100%

TABLE 17b
CD34 cell recovery and viability after being infused through the catheters

| | | 1 | | 2 | | 3 | |
|---|---|---|---|---|---|---|---|
| Catheter | | | R'd | | R'd | | R'd |
| used | Condition | Recoverya | viabb | Recovery | viab | Recovery | viab |
| Raptor | After^{1st} | 116.49% | −3.48% | 121.62% | 12.91% | 110.89% | −7.91% |
| | infusion | | | | | | |
| | After^{2nd} | 91.66% | 1.53% | 85.18% | −23.26% | 122.47% | 1.71% |
| | infusion | | | | | | |
| Sprinter | After^{1st} | 89.19% | −14.66% | 83.34% | −11.83% | 102.72% | −3.29% |
| | infusion | | | | | | |
| | After 2nd | 103.52% | 1.61% | 99.82% | 0.01% | 82.11% | −0.89% |
| | infusion | | | | | | |
| Voyager | After^{1st} | 81.02% | −15.67% | 96.08% | −5.84% | 90.16% | −2.00% |
| | infusion | | | | | | |
| | After^{2nd} | 106.48% | 6.56% | 81.66% | 1.74% | 95.04% | 4.58% |
| | infusion | | | | | | |
| Maverick | After^{1st} | 76.99% | −10.50% | 101.79% | −10.21% | 98.62% | −11.46% |
| | infusion | | | | | | |
| | After 2nd | 228.70% | −1.05% | 88.66% | 2.65% | 103.35% | 9.70% |
| | infusion | | | | | | |

Average of all catheters:

| | | 4 | | 5 | | Average | |
|---|---|---|---|---|---|---|---|
| Catheter | | | R'd | | R'd | Recovery | R'd viab |
| used | Condition | Recovery | viab | Recovery | viab | (SD) | (SD) |
| Raptor | After^{1st} | 97.55% | −8.06% | 96.14% | −3.97% | 108.54% | −2.10% |
| | infusion | | | | | (45.46%) | (7.79%) |
| | After^{2nd} | 111.33% | 9.21% | 98.96% | 1.78% | 101.92% | −1.81% |
| | infusion | | | | | (43.73%) | (11.14%) |
| Sprinter | After^{1st} | 84.57% | −8.39% | 88.65% | −0.28% | 89.69% | −7.69% |
| | infusion | | | | | (37.26%) | (6.16%) |
| | After 2nd | 114.87% | 12.05% | 100.45% | 4.84% | 100.15% | 3.52% |
| | infusion | | | | | (42.22%) | (4.90%) |
| Voyager | After^{1st} | 82.73% | 4.82% | 89.32% | 14.46% | 87.86% | −0.85% |
| | infusion | | | | | (36.28%) | (10.13%) |
| | After^{2nd} | 94.81% | −0.75% | 91.01% | −10.23% | 93.80% | 0.38% |
| | infusion | | | | | (39.12%) | (5.86%) |
| Maverick | After^{1st} | 112.58% | −4.96% | 96.05% | 0.18% | 97.21% | −7.39% |
| | infusion | | | | | (41.34%) | (5.34%) |
| | After 2nd | 89.35% | 6.31% | 117.63% | −5.12% | 125.54% | 2.50% |
| | infusion | | | | | (73.48%) | (5.33%) |

| | | |
|---|---|---|
| Average of all catheters: | 100.59% | −1.68% |

^{a}Recovery of CD34+ cells = (# of CD34 cells after infusion ÷ # of CD34 before infusion) × 100%
^{b}Reduction of CD34+ cell viability = [1 − (CD34+ cell viability % after infusion ÷ CD34+ cell viability % before infusion)] × 100%

Collectively these experiments demonstrate that the serial passage of a chemotactic hematopoietic stem cell product comprising CD34+ cells through a catheter with an internal diameter of at least about 0.36 mm does not adversely affect either catheter integrity or CD34+ cell potency, i.e., CD34+ cell viability, CFU colony growth, or CD34+CXCR+ mediated migratory capacity/mobility.

Study 4: Compatibility of the Cell Product with the Catheters

To further test the compatibility of the chemotactic hematopoietic stem cell product comprising CD34+ cells with each of the catheters that may be used for delivery of the cell product, cell products were tested after multiple passages through each catheter type to evaluate the effects of extreme conditions of stress that would be greater than those expected during the treatment protocol.

At 48 hours post-MMH harvest, the chemotactic hematopoietic stem cell product comprising a range of about 5.73×10⁶ CD34+ cells to about 21.10×10⁶ CD34+ cells (i.e., dosages reflective of the treatment cohort) obtained from individual donors was infused sequentially through three catheters of the same brand, one type of catheter for each donor (Sprinter, Voyager or Maverick), and the cell product assessed for CD34+ cell recovery, colony formation and viability.

TABLE 18
CD34+ cell recovery and sterility after
sequential infusions through the catheters.

| | | Catheter used | | |
|---|---|---|---|---|
| Condition | Parameter | Sprinter | Voyager | Maverick |
| Pre-infusion | CD34+ cell yield | 9.72 × 10⁶ | 2.11 × 10⁷ | 5.73 × 10⁶ |
| After 1st | CD34+ cell recovery | 111% | 103% | 99% |
| catheter | | | | |
| After 2nd | CD34+ cell recovery | 94% | 104% | 97% |
| catheter | | | | |
| After 3rd | CD34+ cell recovery | 99% | 99% | 106% |
| catheter | | | | |
| | Sterility (aerobic and | Negative | Negative | Negative |
| | anaerobic microbes) | | | |

As shown in Table 18, viable, colony forming cells were recovered in all experiments for all three catheters tested (cell recovery 99%, 99% and 106%).

As shown in Table 19, the average viability of the CD34+ cells after passing through the third catheter was 94.000% (based on a range of 93.55%-94.40%) versus 96.01% (based on range of 94.18%-97.93%) of the pre-infusion cell product.

TABLE 19
CD34+ cell viability after sequential
infusions through the catheters.

| | CD34+ cell viability | | | |
|---|---|---|---|---|
| Condition | Sprinter | Voyager | Maverick | Average |
| Pre-infusion | 94.18% | 95.91% | 97.93% | 96.01% |
| After 1^{st} catheter | 94.73% | 96.31% | 95.45% | 95.50% |
| After 2^{nd} Catheter | 95.34% | 95.72% | 95.01% | 95.36% |
| After 3^{rd} catheter | 93.55% | 94.40% | 94.04% | 94.00% |

As shown in Table 20, colony forming unit (CFU) growth derived from the CD34+ cells after passing through the third catheter was 95.27% (based on a range of 43.47%-163.64%) of the infusion product (i.e., the infused chemotactic hematopoietic stem cell product comprising CD34+ cells).

TABLE 20
CFU growth of CD34+ cells after
sequential infusions through the catheters.

| | CFU per 100 CD34+ cells cultured | | |
|---|---|---|---|
| Condition | Sprinter | Voyager | Maverick |
| Pre-infusion | 30.5 | 11.5 | 11.0 |
| After 1st catheter | 22.0 | 14.0 | 22.0 |
| After 2nd catheter | 20.5 | 4.0 | 19.0 |
| After 3rd catheter | 24.0 | 5.0 | 18.0 |
| Recovery from the pre- | 78.69% | 43.47% | 163.64% |
| infused product after the 3rd | | | |
| catheter | | | |

| | |
|---|---|
| Average recovery | 95.27% |

To determine the effect of catheter perfusion on CD34+ cell mobility and ability to grow in culture, a series of experiments were performed where MMH cells obtained from healthy donors were stored at 4° C. for 12 or 24 hours before initiation of Isolex processing. Isolated CD34+ cell product that had been stored for about 12 hours pre-Isolex processing then were stored at 4° C. until about 36 hours had elapsed from the end of processing, for a total of about 48 hours post MMH. At that time they were assessed for SDF-1/CXCR-4 mobility and CFU growth pre- and post-perfusion through a 0.36 mm inner diameter (i.d.) balloon catheter. Similarly, cells that were stored pre-Isolex processing for 24 hours then were stored at 4° C. until 48 hours had elapsed from the end of Isolex processing, for a total of 72 hours, and then ssessed.

TABLE 21
12 inbound/48 outbound and 48 hour inbound/72 hour outbound
from MMH: SDF-1/CXCR-4 mobility (% population of migrated CD34+
cells) and CFU (per 100 viable CD34+ plated) pre catheter
perfusion (“PRE”) and post catheter perfusion (“POST”)

Time (h)

| after MMH | SDF-1/CXCR4 mobility (%) // # of CFU per | | | | |
|---|---|---|---|---|---|
| Inbound/ | 100 viable CD34+ cells plated | | | | |
| outbound | A | B | C | D | E |
| 12/48 | 2.7 // 14 | 8.8 // 15 | 15.8 // 16 | — | — |
| PRE | | | | | |
| 12/48 | 3.4 // 15 | 18.9 // 13 | 17.6 // 8 | — | — |
| POST | | | | | |
| 24/72 | — | — | — | 34 // 37 | 18.9 // 27.5 |
| PRE | | | | | |
| 24/72 | | | | 34 // 43 | 23.5 // 24 |
| POST | | | | | |

The results in Table 21 demonstrate that neither CD34+ CXCR-4-mediated cell mobility nor the cell's ability to grow in culture at any of the time points tested was affected adversely by perfusion through a catheter having an internal diameter of at least 0.36 mm.

The stabilizing effect of serum.

The following data confirm the importance of the stabilizing effect of serum to the migratory capability of the selected CD34+ cells.

As shown in Table 22, no CXCR-4 migratory activity was observed for all samples tested including the pre-catheter infusion samples when the composition comprising a chemotactic hematopoietic stem cell product was formulated without serum.

TABLE 22
Chemotaxis of CD34+ cells after sequential infusions
through the catheters in the absence of serum.

| | Migration (%) | | |
|---|---|---|---|
| Condition | Sprinter | Voyager | Maverick |
| Pre-infusion | 0.0 | 0.0 | 0.1 |
| After 1st catheter | 0.0 | 0.0 | 0.0 |
| After 2nd catheter | 0.0 | 0.0 | 0.1 |
| After 3rd catheter | 0.0 | 0.0 | 0.0 |

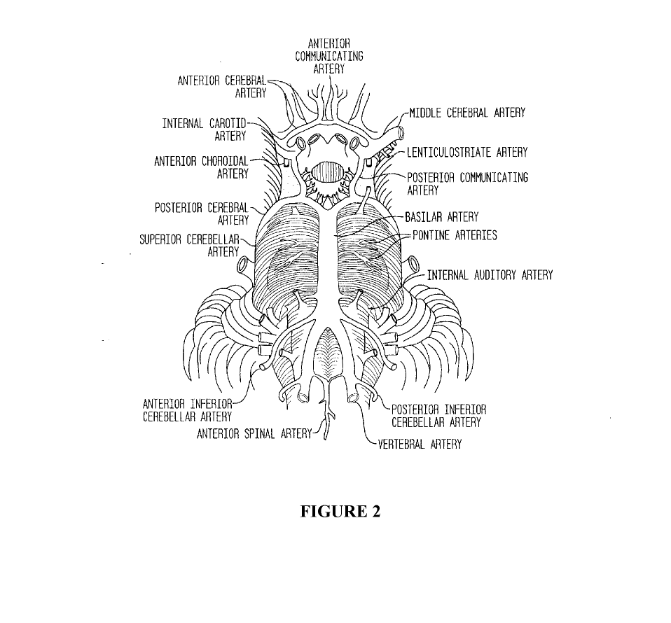

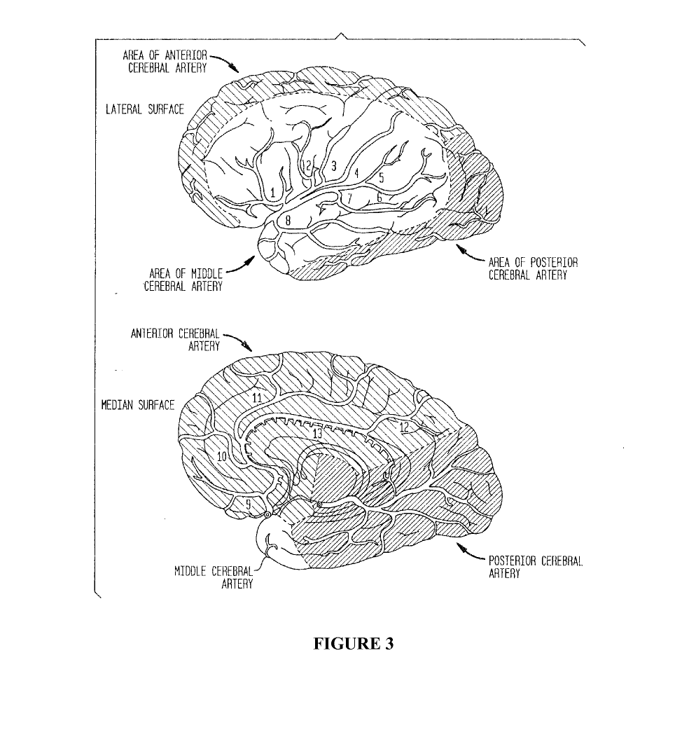

FIGS. 2 and 3 further illustrate that Isolex selected CD34+ cells retain their migratory capacity longer when formulated in the presence of human serum. Following Isolex processing, the bone marrow derived hematopoietic stem cell product comprising selected CD34+ cells was formulated either in (1) phosphate buffered saline (Dulbecco's phosphate buffered saline, Ca^{2+,} Mg² Free (Baxter Cat. No. EDR9865) (“PBS”) containing 1% human serum albumin, 25 U/ml of heparin sodium and various concentrations (about 0%, about 10%, about 20%, or about 70%) of autologous serum; or (2) normal saline (0.9%) containing 1% human serum albumin, 25 U/ml of heparin sodium and (about 0% or about 10%) autologous serum. SDF-1/CXCR-4 mediated CD34+ cell migratory capacity was evaluated at different times during final product storage (at 2° C.-8° C.) and after passing the cells through the catheter at the same rate and duration as anticipated by the clinical protocol. None of these formulations affected CD34+ cell viability or the recovery of CD34+ cells after they had been passed through the catheter.

Regardless of whether the chemotactic hematopoietic cell products comprising selected CD34+ cells was (i) formulated either in PBS-serum or in saline-serum and (ii) either passed through the catheter immediately or passed through the catheter after a prolonged stability testing storage interval at about 4° C. to about 8° C., they maintained an average of 96.6% viability (range 92.5%-98.6%) and an average CXCR-4-mediated migratory capacity of 11.4% (range 2.4%-30.6%), representing a total time from harvest to mobility analysis of up to 48 hours.

As shown in FIG. 2 panel (a), cells formulated in PBS alone at about 25 hours retained about 10% of their CXCR-4 migratory capacity, which dropped off to near 0 at about 48 hours. As shown in panel (b), cells formulated in normal saline alone retained little, if any, of their migratory capacity. As shown in panels (c) and (d), cells formulated with PBS containing at least about 10% serum retained about 10-15% of their migratory capacity for up to about 55 hours (c), while cells formulated with saline and at least about 10% serum retained about 20% of their migratory capacity for up to about 50 hours. As shown in panels (e) and (f), cells retained a higher migratory capacity for a longer duration in PBS supplemented with even higher concentrations of serum.

As shown in FIG. 3, the product of the present invention comprising selected CD34+ cells when formulated in 10% serum, retained 14.25%, <1%, 6%, and 5.8% of its CD34+/CXCR-4 mediated migratory capacity about 24, about 32, about 48 and about 56 hours after harvest, respectively. FIG. 3 further shows that the product of the present invention comprising selected CD34+ cells when formulated in 20% serum retained 18.25%, 10.25%, 17% and 11% of its CD34+/CXCR-4 mediated migratory capacity about 24, about 32, about 48 and about 56 hours after harvest, respectively. The term “stabilizing amount” as used herein therefore refers to the amount of serum that, when included in the formulation of the product of the present invention comprising selected CD34+ cells, enables these cells to retain their CXCR-4 mediated chemotactic activity and hematopoietic colony forming ability.

Study 5: Final Product Sterility Testing

Due to the limited yield of CD34+ cells obtained from a 300-ml MMH, final cell product sterility will be assessed using the supernatant removed from the final product formulation in order to preserve cell product for infusion. Supernatant samples are loaded into the syringes in a manner identical to that used to load the cell product into the syringes used for infusion (see supra). To demonstrate that such a sample will be representative of the final cell product formulation, selected CD34+ cells were innoculated in infusion solution prior to centrifugation of the final product with C. sporogenes (13 CFU/ml), P. aeruginosa (2 CFU/ml), S. aureus (18 CFU/ml), A. niger (17 CFU/ml), C. albicans (3 CFU/ml) and B. subtilis (17 CFU/ml) (See table 22). After centrifugation, the sterility of both cell pellet and non-cell supernatant fractions was assessed using USP aerobic and anaerobic testing.

TABLE 23
Bacteria and fungi used for the sterility study. Each
source microorganism vial prepared by Microbiological
Environments contained 400 microbes per ml, but the numbers
of CFU derived from each species are varied.

| | | | Expected CFU/ml of |
|---|---|---|---|
| | Total # of | Total | inoculated sample |
| Microbe | microbes/ml | CFU/ml | (21 ml) |
| C. sporogenes | 400 | 279 | 13 |
| P. aeruginosa | 400 | 36 | 2 |
| S. aureus | 400 | 371 | 18 |
| A. niger | 400 | 356 | 17 |
| C. albicans | 400 | 62 | 3 |
| B. subtilis | 400 | 349 | 17 |

As shown in Table 24, both the cell pellet fraction and suspension fractions from all tested samples showed outgrowth of the inoculated microorganisms, while un-inoculated controls showed no growth. Further, no apparent differential growth rate was observed between testing of cell pellet fractions and the suspension fractions for all microorganisms tested. Samples taken before each step of the processing procedure and following the final perfusion through the catheters all tested negative for microbial contamination.

TABLE 24
14-day sterility testing of nucleated cell (NC)
samples inoculated with specific species of microorganism
(400 microbes in 21-m1 NC sample).

| Sample | | | | | |
|---|---|---|---|---|---|
| with microbe | Medium | Sample | | | |
| Inoculated | type | fraction | Test 1 | Test 2 | Test 3 |
| C. sporogenes | FTM^{a} | Cell pellet | Positive | Positive | Positive |
| | | Suspension | Positive | Positive | Positive |
| S. aureus | FTM | Cell pellet | Positive | Positive | Positive |
| | | Suspension | Positive | Positive | Positive |
| P. aeruginosa | FTM | Cell pellet | Positive | Positive | Positive |
| | | Suspension | Positive | Positive | Positive |
| A. niger | TSB^{b} | Cell pellet | Positive | Positive | Positive |
| | | Suspension | Positive | Positive | Positive |
| C. albicans | TSB | Cell pellet | Positive | Positive | Positive |
| | | Suspension | Positive | Positive | Positive |
| B. subtilis | TSB | Cell pellet | Positive | Positive | Positive |
| | | Suspension | Positive | Positive | Positive |

| | | | |
|---|---|---|---|
| Positive control: | FTM | Cell | Positive |
| C. sporogenes | | suspension | |
| Positive control: | FTM | | Positive |
| S. aureus | | | |
| Positive control: | FTM | | Positive |
| P. aeruginosa | | | |
| Positive control: | TSB | | Positive |
| A. niger | | | |
| Positive control: | TSB | | Positive |
| C. albicans | | | |
| Positive control: | TSB | | Positive |
| B. subtilis | | | |
| Negative control: | FTM | Cell | Negative |
| No microbes | | suspension | |
| Negative control: | TSB | | Negative |
| No microbes | | | |

^{a}Fluid thioglycollate medium
^{b}Tryptic soy broth

Preclinical Study Summary

Collectively, these preclinical data indicate that the manufacturing and testing procedures described are capable of generating adequate numbers of viable cells with adequate stability to withstand shipment and perfusion through the catheter in a manner that should pose no additional safety concerns to the subject other than those associated with the routine use of fluid infusion through the balloon catheter.

### Example 9

### Percussive Injury to Model Traumatic Brain Injury (TBI)

The therapeutic effectiveness of an isolated, nonexpanded population of autologous mononuclear cells comprising a population of CD34+ cells, which further contains a subpopulation of potent SDF-1 mobile CD34+/CXCR-4 cells that have CXCR-4-mediated chemotactic activity in treating a vascular insufficiency following a traumatic brain or spinal cord injury will be tested in animal models of traumatic brain injury as described previously (Lu, D. et al., Neuroreport, 12(3): 559-563, 2001; Li, S. et al., Brain Research, 1444, 76-68, 2012, the entire contents of each reference is incorporated by reference herein).

Briefly, rats will be anesthetized using 10% chloride hydrate (3.0 ml/kg) via intraperitoneal injection and placed in a stereotaxic device. The animal's body temperature will be maintained using a heading pad (about 37° C.). The incision area will be shaved and sterilized using Betadine® and 70% ethanol. A midline incision will be made into the scalp and fascia of the head using aseptic techniques. A right parietal craniectomy (diameter 3.8 mm) will be drilled 3.0 mm lateral to the sagittal suture and 4.0 mm posterior to the coronal suture with the dura left intact. Through this craniectomy, acoustic, percussive pressure will be applied to result in an injury to cerebral tissue to model traumatic brain injury.

Animals will be randomized into groups permitting the following approaches:

1. Assessment of SDF-1 levels in the infarct and peri-infarct region;

2. No intervention;

3. Administration of CD34+CXCR4+ cells in one or more doses in one or more volumes of diluents via direct injection into the traumatic penumbra of the injured brain or spinal cord parenchyma.

4. Administration of CD34+CXCR4+ cells in one or more doses in one or more volumes of diluents via intravascular administration.

For each animal/group the degree of dysfunction will be assessed using standard scoring tools over a period of weeks to months, including metrics such as the Modified Neurological Severity Score and the Morris Water Maze Test.

The Modified Neurological Severity Score (mNSS)

One of the most common neurological scales used in animal studies is the Modified Neurological Severity Score (mNSS). The mNSS rates neurological functioning on a scale of 14 or 18, depending on mice or rat, respectively. The mNSS includes a composite of motor (muscle status and abnormal movement), sensory (visual, tactile and proprioceptive), reflex, and balance tests. One point is given for the inability to perform each test while one point is deducted for the lack of a tested reflex, and an overall composite score is given to determine impairment. Neurological rating scores have the ability to assess multiple deficits and can be good for testing over periods of 30-60 days.

Post-injury neurologic impairment of the treated animals will be graded on a scale of 0 to 18 (normal score, 0; maximal deficit score, 18) by an investigator who will be blinded to the experimental groups before and at 1, 3, 7, 14, and 21 days after injury as described previously (Chen et. al., Stroke, 32: 2682-2688, 2001, incorporated by reference in its entirety).

Morris Water Maze Test

The Morris Water Maze (MWM) is a test of spatial learning for rodents that relies on distal cues to navigate from start locations around the perimeter of an open swimming arena to locate a submerged escape platform. Spatial learning is assessed across related trials and reference memory is determined by preference for the platform area when the platform is absent. Reversal and shift trials enhance the detection of spatial impairments. Trial-dependent, latent and discrimination learning can be assessed using modifications of the basic protocol. Search-to-platform area determines the degree of reliance on spatial versus non-spatial strategies. Cued trials determine whether performance factors that are unrelated to place learning are present. Escape from water is relatively immune from activity or body mass differences, making it ideal for many experimental models. The MWM has proven to be a robust and reliable test that is strongly correlated with hippocampal synaptic plasticity and NMDA receptor function (Vorhees and Williams, Nat. Protco. 1, 848-858, 2006).

Spatial reference memory of the treated animals will be evaluated via Morris Water Maze (MWM) (Morris, J. Neurosci. Methods, 11: 47-60, 1984) according to the protocol described previously (Chen et al., J. Neurotrauma, 26: 253-260 2009; Vorhees and Williams, Nat. Protco. 1, 848-858, 2006). A circular open-field tank (diameter 150 cm, height 50 cm) will be filled with opaque water at 22(±1)° C. and a plexiglass cylinder platform will be submerged 2 cm below water level at the same location-in the middle of southwest (SW) quadrant. On post-surgery day 14, rats with traumatic brain injury (TBI) administered with vehicle (TBI+control) and rats with TBI administered with CD34+CXCR4+ hematopoietic stem cell product (TBI+test) (n=6/group) will be placed into the maze for 1 min/day for three days to acclimatize the trial without platform located. Spatial acquisition and reference memory trials will be performed the next six consecutive days. At spatial acquisition phase, four trials will be tested each day for five days. Rats will be placed into the water facing the side wall at one of four starting locations (north, south, northwest, southeast) and will be trained to find the submerged platform by visual distal cues within a maximum swimming time of 120 s. If the platform is found within 120 s by a rat, the rat will be allowed to stay on it for 30 s. If the rat fails to find the platform within the given time, the rat will be guided to the platform and be allowed to remain on the platform for 30

s. The latency and swim speed will be monitored via a tracking system (Ethovision 3.0, Noldus Information Technology, Wageningen, Netherlands). The latency of getting to the submerged platform each day will be calculated and a spatial learning curve will be plotted based on the data from the trials. 24 h after the last acquisition trial, rats who underwent reference memory trials will be removed; put into the maze at the northeast position (NE); and will be allowed to swim for 30 s. Swim speed and the time spent in the goal quadrant (the quadrant where the platform was previously located) will be calculated from reference memory trials aimed to investigate how the task had been learned.

### Example 10

### Evaluation of the Effects of Autologous, Chemotactic Hematopoietic Stem Cells in Human Traumatic Brain Injury Patients

For human studies, patients suffering from traumatic brain injury (TBI) will be treated in the acute phase within the first days to weeks after TBI.

The chemotactic hematopoietic stem cell product comprising a nonexpanded isolated population of autologous mononuclear cells will be administered to patients who have suffered or are suffering from at least one incident of traumatic brain or spinal cord injury. According to some embodiments, the mononuclear cells are enriched for CD34+ cells. The CD34+ cells further contain a subpopulation of potent SDF-1 mobile CD34+/CXCR-4 cells that have CXCR-4-mediated chemotactic activity.

Patients qualified for treatment will be selected based on their cognitive function (e.g., Glasgow Coma Scale (GCS)), outcome measures for various spinal cord injuries (e.g., pain, sensory/motor strength, gait/ambulation, spasticity), clinical status, and the presence of an infarcted region in the brain or spinal cord as determined by neuroimaging.

In order to assess the short-term and long-term effect of the hematopoietic stem cell product of the present invention in the treatment of the injured brain or spinal cord, patients will be categorized into three groups:

(1) patients who have experienced a severe traumatic brain injury (TBI), for example, patients with an acute subdural hematoma or a parenchymal hematoma, which increases intracranial pressure, that requires craniectomy;

(2) patients who have experienced a mild or mild-to-moderate closed head trauma (for example, a cerebral concussion, a cerebral contusion, an epidural hematoma, a subdural hematoma, an intraventricular hemorrhage, and a diffuse axonal injury) but does not require craniectomy based on neuroimaging; and

(3) patients who have experienced recurrent concussive brain injury and have a risk of developing chronic traumatic encephalopathy (CTE).

The administration routes (e.g., direct injection into the penumbra of the injured brain or spinal cord tissue and intravascular administration), volume, and infusion rate of the hematopoietic stem cell product of the present invention will be determined based on various factors, including, but not limited to: (1) distance between the injured brain or spinal cord parenchyma and the site of infusion; (2) the extent of the parenchymal injury of the brain or spinal cord; (3) expression level of SDF-1 in the penumbra of the injured brain or spinal cord parenchyma; and (4) the migration rate of the infused CD34+/CXCR-4+ cells into the injured brain or spinal cord parenchyma.

For direct injection of the hematopoietic stem cell product into the injured brain or spinal parenchyma, the hematopoietic stem cell product of the present invention will be delivered via stereotactically or impedance-guided injection. The injection will be performed by utilizing either new or existing access (e.g., a burr hole in the cranium typically used for monitoring of intracranial pressure) or via a route made available after craniectomy.

For intravascular delivery, vascular access will be obtained by standard techniques. All subjects will receive standard medications during the catheterization procedure in accordance with routine practice. Once vascular access has been established, the chemotactic hematopoietic stem cell product will be infused into an artery (for example, carotid artery, cerebral artery, femoral artery, brachial artery or a branch thereof).

Clinical variables of administration, e.g., optimal doses for intravascular infusion or direct injection will be tested. The intravascular injection will include assessment of the importance of distance from the lesion, i.e., administration into carotid vs. cerebral artery vs. 3^{rd} or 4^{th} generation vessels, as well as the assessment of whether complete or partial occlusion of blood flow following injection is necessary.

Following infusion of the hematopoietic stem cell product, the short-term and long-term prognosis of the traumatic brain or spinal cord injury in the treated patients will be monitored by examining the patient's cognitive functions (e.g., by applying Glasgow Coma Scale (GCS) or Modified Neurological Severity Score (mNSS)) or outcome measures for various spinal cord injury (e.g., pain, sensory/motor strength, gait/ambulation, spasticity), or by examining the extent of ischemic parenchyma in the injured brain or spinal cord via neuroimaging.

While the described invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the described invention. All such modifications are intended to be within the scope of the claims appended hereto.

We claim:

1. A method for treating a vascular insufficiency of a cerebral artery, spinal cord artery, or a branch thereof following a traumatic injury to head or spine that results in an injury to paranchyma of brain, spinal cord, or both, the method comprising:
(a) administering to a subject in need thereof via a delivery device a therapeutic amount of a pharmaceutical composition comprising:
(i) a therapeutic amount of a sterile chemotactic hematopoietic stem cell product containing an isolated, nonexpanded population of autologous mononuclear cells comprising a subpopulation of CD34+ cells;
wherein the pharmaceutical composition is formulated for administration parenterally;
characterized in that the isolated population of mononuclear cells comprising a subpopulation of CD34+ cells further contains a subpopulation of potent SDF-1 mobile CD34+/CXCR-4+ cells that have CXCR-4-mediated chemotactic activity, such that the therapeutic amount comprises at least 0.5×10⁶ potent SDF-1-mobile CD34+CXCR-4+ cells that have CXCR-4-medicated chemotactic activity; and
further characterized as having the following properties for at least 24 hours following acquisition of the chemotactic hematopoietic stem cell product when tested in vitro after passage through a catheter:
(1) at least 70% of the cells are CD34+ cells;
(2) retains at least 2% of the CXCR-4-mediated chemotactic activity of the subpopulation of subpopulation of potent SDF-1 mobile CD34+/CXCR-4+ cells that have CXCR-4-mediated chemotactic activity measured prior to purification;
(3) is at least 70% viable; and
(4) is able to form hematopoietic colonies in vitro; and
(ii) a stabilizing amount of serum, which is effective to retain the CXCR-4-mediated chemotactic activity and hematopoietic colony forming activity of the population of SDF-1 mobile CD34+CXCR-4+ cells from acquisition to infusion; and
(b) monitoring the subject's cognitive and neurologic functions,
wherein the therapeutic amount of the sterile chemotactic hematopoietic stem cell product is effective to improve perfusion and to preserve existing nerve cells and their function in an area of ischemia in the paranchyma of the injured brain, spinal cord or both.

2. The method according to claim 1, wherein the traumatic injury is a closed head injury, a concussive head injury, a penetrating head injury, or a combination thereof.

3. The method according to claim 1, wherein the traumatic injury is a severe traumatic brain injury that requires craniectomy.

4. The method according to claim 3, wherein the severe traumatic brain injury comprises an acute subdural hematoma or a parenchymal hematoma.

5. The method according to claim 1, wherein the traumatic injury is a mild to moderate closed head injury that does not require craniectomy.

6. The method according to claim 5, wherein the mild to moderate closed head injury is selected from the group consisting of cerebral concussion, cerebral contusion, epidural hematoma, subdural hematoma, intraventricular hemorrhage, and diffuse axonal injury.

7. The method according to claim 1, wherein the injury to the brain, spinal cord, or both comprises an infarct area injury selected from the group consisting of apoptotic nerve cell loss in the infarct area; adverse remodeling after an acute cerebral infarction, when compared to controls; a progressive decline in cognitive function following the acute cerebral infarction; hypoperfusion of at least one ischemic peri-infarct zone; and a combination thereof.

8. The method according to claim 7, wherein the method is capable of improving microvascular blood flow in the infarct area, of decreasing area of the infarct injury, of decreasing infarct mass, of increasing perfusion of at least one ischemic peri-infarct zone of nerve tissue, or a combination thereof when compared to controls.

9. The method according to claim 7, wherein the chemotactic hematopoietic stem cell product is administered after peak inflammatory cytokine cascade production in an infarcted area and before completion of scar formation in the infarcted area.

10. The method according to claim 1, wherein the injury to the brain, spinal cord, or both, places the subject at risk for developing chronic traumatic encephalopathy (CTE) of the brain, spinal cord or both.

11. The method according to claim 8, wherein the chronic traumatic encephalopathy (CTE) is associated with progressive tauopathy in the injured brain, spinal cord or both.

12. The method according to claim 1, wherein the pharmaceutical composition further comprises a therapeutic amount of at least one compatible therapeutic agent.

13. The method according to claim 10, wherein the therapeutic amount of the compatible therapeutic agent is capable of promoting function of existing nerve cells to compensate for loss of function due to neuronal death, of regenerating new nerve cells, or both.

14. The method according to claim 10, wherein the compatible therapeutic agent comprises a vasoactive agent, an anticoagulant agent, an antiplatelet agent, an antihypercholesterolemic agent, or a combination thereof.

15. The method according to claim 12, wherein the anti-coagulant agent is selected from the group consisting of a coumarin, heparin, an inhibitor of Factor Xa, batroxobin, hementin, and a combination thereof.

16. The method according to claim 12, wherein the delivery device is coated with the anticoagulant agent.

17. The method according to claim 10, wherein the compatible therapeutic agent comprises a cytokine, a placental growth factor, granulocyte colony-stimulating factor, macrophage colony-stimulating factor, a vascular endothelial growth factor, neuregulin-1, tumor necrosis factor-like weak inducer of apoptosis, or a combination thereof.

18. The method according to claim 15, wherein the cytokine is at least one selected from the group consisting of vascular endothelial growth factor (VEGF), placental growth factor (PIGF), granulocyte colony-stimulating factor (G-CSF), and macrophage colony-stimulating factor (M-CSF).

19. The method according to claim 15, wherein the vascular endothelial growth factor is selected from the group consisting of VEGF-A, VEGF-B, VEGF-C, and VEGF-D.

20. The method according to claim 15, wherein the at least one compatible therapeutic agent is placental growth factor.

21. The method according to claim 1, wherein the administering parenterally is by direct injection or by infusion into the paranchyma of the injured brain, spinal cord, or both.

22. The method according to claim 19, wherein the therapeutic amount of the chemotactic hematopoietic stem cell product is administered via stereotactically-guided direct injection.

23. The method according to claim 19, wherein the therapeutic amount of the chemotactic hematopoietic stem cell product is administered via impedance-guided direct injection.

24. The method according to claim 1, wherein the administering parenterally is performed intravascularly.

25. The method according to claim 22, wherein the pharmaceutical composition is infused into an artery or a branch thereof.

26. The method according to any one of claims 22 and 23, wherein the administering parenterally is performed at one or more infusion dates.

27. The method according to claim 23, wherein the artery is a carotid artery or a branch thereof.

28. The method according to claim 23, wherein the artery is a cerebral artery or a branch thereof.

29. The method according to claim 1, wherein the delivery device is a catheter.

30. The method according to claim 27, wherein the catheter comprises an anticoagulant agent, a material that acts as an anticoagulant, or both.

31. The method according to claim 27, wherein the catheter has an internal diameter of at least 0.36 mm.

32. The method according to claim 1, wherein the stabilizing amount of serum is from about 0.1% to about 70% (v/v).
